(19) United States
(12) Patent Application Publication
(10) Pub. No.: US 2021/0179620 A1
(43) Pub. Date: Jun. 17, 2021

(54) MESOIONIC IMIDAZOLIUM COMPOUNDS AND DERIVATIVES FOR COMBATING ANIMAL PESTS

(71) Applicant: BASF SE, Ludwigshafen (DE)

(72) Inventors: Joachim DICKHAUT, Ludwigshafen (DE); Ashokkumar ADISECHAN, Navi Mumbai (IN); Gopal Krishna DATTA, Goettingen (DE); Olesya KUZMINA, Ludwigshafen (DE)

(21) Appl. No.: 16/621,656

(22) PCT Filed: Jun. 14, 2018
(86) PCT No.: PCT/EP2018/065828

§ 371 Date: Dec. 11, 2019

(30) Foreign Application Priority Data
Jun. 16, 2017 (IN) 201721021122
Sep. 6, 2017 (EP) 17189535.2

(51) Int. Cl. C07D 487/04; C07D 498/04; C07D 513/04; C07D 471/04; A01N 43/90

(52) U.S. Cl. CPC C07D 487/04; C07D 498/04; A01N 43/90; C07D 471/04; C07D 513/04

(57) ABSTRACT

The present invention relates to compounds of formula (I),

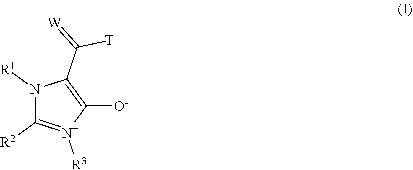

wherein W, T, R¹, R² and R³ are defined as in the description, and to the stereoisomers, salts, tautomers and N-oxides thereof and to compositions comprising such compounds.

The invention also relates to methods and uses of these compounds and of compositions thereof, for combating and controlling animal pests. Furthermore the invention relates also to pesticidal methods of applying such substituted pyrimidinium compounds.

The present invention relates to insecticidal substituted imidazolium compounds and/or to the compositions comprising such compounds for combating invertebrate pests. The invention also relates to pestiddal methods, to uses and to applications of substituted imidazolium compounds as described in the present invention and the stereoisomers, salts, tautomers and N-oxides thereof as well as compositions comprising them.

Invertebrate pests and in particular insects, arthropods and nematodes destroy growing and harvested crops and attack wooden dwelling and commercial structures, thereby causing large economic loss to the food supply and to property. While a large number of pesticidal agents are known, due to the ability of target pests to develop resistance to said agents, there is an ongoing need for new agents for combating invertebrate pests such as insects, arachnids and nematodes. It is therefore an object of the present invention to provide compounds having a good pesticidal activity and showing a broad activity spectrum against a large number of different invertebrate pests, especially against difficult to control insects, arachnids and nematodes.

It has been found that these objectives can be achieved by compounds of the general formula (I), as defined below, including their stereoisomers, their salts, in particular their agriculturally or veterinary acceptable salts, their tautomers and their N-oxides.

Therefore, in a first aspect the present invention provides compounds of formula (I)

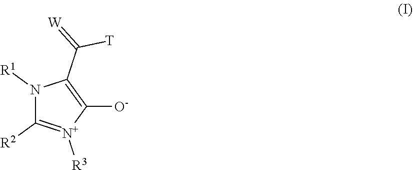

wherein
W is O, and
T is R⁵, OR⁶, —N(R⁷)(R⁸) or —N(R^{7a})—N(R⁷)(R⁸);
or
W is S, and
T is —N(R⁷)(R⁸) or —N(R^{7a})—N(R⁷)(R⁸);
R¹ is NO₂, CN C₁-C₈-alkyl, C₂-C₈-alkenyl, C₂-C₈-alkynyl, C₃-C₁₀-cycloalkyl, C₄-C₁₀-cycloalkenyl, C₅-C₁₄-cycloalkylcycloalkyl or R¹ may form a three- to eleven-membered saturated, or partially unsaturated or aromatic carbo- or heterocyclic ring or ring system, which may contain 1 to 4 heteroatoms selected from N(R^{c})ₚ, O, and S, wherein S may be oxidized, and wherein the aforementioned groups and the carbo- or heterocyclic rings system may be unsubstituted, partially or fully substituted with R^{a}; or R¹ is C(═O)R^{b}, C(═O)OR^{e}, NR^{b}R^{c}, C(═O)NR^{b}R^{c}, C(═S)NR^{b}R^{c}, SO₂NR^{b}R^{c}, OC(═O)R^{c}, OC(═O)OR^{e}, OC(═O)NR^{b}R^{e}, N(R^{c})C(═O)R^{c}, N(R^{c})C(═O)OR^{e}, N(R^{c})C(═O)NR^{b}R^{c}, NR^{c}SO₂R^{b}, NR^{c}SO₂NR^{b}R^{c}, Si(R^{d})₃, C(═NR^{c})R^{c}, C(═NOR^{c})R^{c}, C(═NNR^{b}R^{c})R^{c}, C(═NN(C(═O)R^{b})R^{c})R^{c}, C(═NN(C═O)OR^{c})(R^{c})₂, S(═O)ₒ(═NR^{b})_{q}R^{c} or N═CR^{b}R^{c};
R² and R³ are each independently selected from
   C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with R^{a}; or C(═O)R^{b}, C(═O)OR^{e}, NR^{b}R^{c}, C(═O)NR^{b}R^{c}, C(═S)NR^{b}R^{c}; C(═NR^{c})R^{c}, C(═NOR^{c})R^{c}, C(═NNR^{b}R^{c})R^{c}, C(═NN(C(═O)R^{b})R^{c})R^{c}, C(═NN(C═O)OR^{c})R^{c})₂, or
   OR^{c}, OC(═O)R^{c}, OC(═O)OR^{e}, OC(═O)NR^{b}R^{e},
   NR^{b}R^{c}, N(R^{c})C(═O)R^{c}, N(R^{c})C(═O)OR^{e}, N(R^{c})C(═O)NR^{b}R^{c}, NR^{c}SO₂R^{b},
   NR^{c}SO₂NR^{b}R^{c}, N═CR^{b}R^{c};
   S(O)ₘR^{b}, SO₂NR^{b}R^{c}, S(═O)ₒ(═NR^{b})_{q}R^{c} or Si(R^{d})₃, or
   a three- to eleven-membered saturated, or partially unsaturated or aromatic carbo- or heterocyclic ring or ring system, which may contain 1 to 4 heteroatoms selected from N(R^{c})ₚ, O, and S, wherein S may be oxidized, wherein the carbo- or heterocyclic rings system may be unsubstituted, partially or fully substituted with R^{a}; or
   wherein one of R² and R³ is R⁴;
wherein one of R² and R³ is substituted with one R⁴;
   or
R² and R³ form a four- to seven-membered ring, taken together with the carbon and nitrogen of the imidazole ring in formula (I), wherein each remaining ring member is selected from carbon atoms and up to 3 heteroatoms independently selected from up to 2 O, up to 2 S, and up to 3 N, wherein up to 2 carbon atom ring members are independently selected from C(═O) and C(═S), and the sulfur atom ring members are independently selected from S(═O)ₘ, wherein each ring member may be substituted with R^{a} and/or R^{c};
wherein the ring is substituted with one R⁴;
R⁴ is Het or R^{4a};
Het is a three- to ten-membered heterocyclic ring or a seven- to eleven-membered heterocyclic ring system, each ring or ring system member selected from carbon atoms and up to 4 heteroatoms independently selected from up to 2 O, up to 2 S, and up to 4 N(R^{c})ₚ, wherein up to 3 carbon atom ring members are independently selected from C(═O) and C(═S) and the sulfur atom ring members are independently selected from S(═O)ₒ(═NR^{b})_{q}, each ring or ring system optionally substituted with up to 5 R^{a}; o, q are each independently 0, 1 or 2, provided that the sum (o+q) is 0, 1 or 2 for each ring;
R^{4a} is C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₇-cycloalkyl, C₃-C₇-halocycloalkyl, C₄-C₈-alkylcycloalkyl, C₄-C₈-haloalkylcycloalkyl, C₄-C₈-cycloalkylalkyl, C₄-C₈-halocycloalkylalkyl, C₁-C₆-alkoxy, C₁-C₆-haloalkoxy, C₁-C₆-alkylcarbonyl, C₁-C₆-haloalkylcarbonyl, CN;
   each optionally substituted with one or more substituents selected from CN, OR⁶, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R⁶, OSO₂NR^{b}R^{c}, NR^{b}SO₂R^{c}, NR^{b}SO₂NR^{b}R^{c}, SF₅, OCN, SCN, Si(R^{d})₃, C(═N(O)ₚR^{b})R^{b}, C(═NNR^{b}R^{c})R^{b}, C(═NN(C(═O)OₚR^{c})R^{b})R^{b}, ON═CR^{b}R^{c}, ONR^{b}R^{c}, S(═O)ₒ(═NR^{b})_{q}R^{c}, SO₂NR^{b}(═O)NR^{b}R^{c}, P(═W)R^{b}R^{c}, OP(═W)(OₚR^{c})R^{b}, OP(═W)(OR^{c})₂, N═CR^{b}R^{c}, NR^{b}N═CR^{b}R^{c}, NR^{b}NR^{b}R^{c}, NR^{b}C(═S)NR^{b}R^{c}, NR^{b}C(═NR^{b})NR^{b}R^{c}, NR^{b}NR^{b}C(═W)NR^{b}R^{c}, NR^{b}NR^{b}SO₂NR^{b}R^{c}, or N═S(═O)ₚR^{c}R^{c}, or
   two geminally bound groups R^{4a} together may form a group selected from ═O, ═S, ═CR^{b}R^{c}, ═NR^{c}, ═NOR^{c}, and ═NNR^{c}R^{c};
or R^{4a} is phenyl optionally substituted with one or more substituents selected from halogen, CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b}SO₂R^{c}, NR^{b}SO₂NR^{b}R^{c}, SF₅, OCN, SCN, Si(R^{d})₃, C(═N(O)ₚR^{b})R^{b}, C(═NNR^{b}R^{c})R^{b}, C(═NN(C(═O)OₚR^{c})R^{b})R^{b}, ON═CR^{b}R^{c}, ONR^{b}R^{c}, S(═O)ₒ(═NR^{b})_{q}R^{c}, SO₂NR^{b}(═O)NR^{b}R^{c}, P(═W)R^{b}R^{c}, OP(═W)(O_{b}R^{c})R^{b}, OP(═W)(OR^{c})₂, N═CR^{b}R^{c}, NR^{b}N═CR^{b}R^{c}, NR^{b}NR^{b}R^{c}, NR^{b}C(═S)NR^{b}R^{c}, NR^{b}C(═NR^{b})NR^{b}R^{c}, NR^{b} NR^{b}C(═W)NR^{b}R^{c}, NR^{b}NR^{b}SO₂NR^{b}R^{c}, or N═S(═O)ₚR^{c}R^{c},
   or R^{4a} is phenyl optionally substituted with one or more substituents selected from C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₇-cycloalkyl, C₃-C₇-halocycloalkyl, C₄-C₈-alkylcycloalkyl, C₄-C₈-haloalkylcycloalkyl, C₄-C₈-cycloalkylalkyl, C₄-C₈-halocycloalkylalkyl, C₁-C₆-alkoxy, C₁-C₆-haloalkoxy, C₂-C₆-alkylcarbonyl, C₂-C₆-haloalkylcarbonyl, which groups may optionally be substituted with halogen, CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b}SO₂R^{c}, NR^{b}SO₂NR^{b}R^{c}, SF₅, OCN, SCN, Si(R^{d})₃, C(═N(O)ₚR^{b})R^{b}, C(═NNR^{b}R^{c})R^{b}, C(═NN(C(═O)OₚR^{c})R^{b})R^{b}, ON═CR^{b}R^{c}, ONR^{b}R^{c}, S(═O)ₒ(═NR^{b})_{q}R^{c}, SO₂NR^{b}(═O)NR^{b}R^{c}, P(═W)R^{b}R^{c}, OP(═W)(OₚR^{c})R^{b}, OP(═W)(OR^{c})₂, N═CR^{b}R^{c}, NR^{b}N═CR^{b}R^{c}, NR^{b}NR^{b}R^{c}, NR^{b}C(═S)NR^{b}R^{c}, NR^{b}C(═NR^{b})NR^{b}R^{c}, NR^{b}NR^{b}C(═W)NR^{b}R^{c}, NR^{b}NR^{b}SO₂NR^{b}R^{c}, or N═S(═O)ₚR^{c}R^{c},
R^{a} is each independently halogen, C₁-C₆-alkyl, C₁-C₆-haloalkyl, C₁-C₆-alkoxy, C₁-C₆-haloalkoxy, C₂-C₄-alkenyl, C₂-C₄-alkynyl, C₃-C₆-cycloalkyl, CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b}SO₂R^{c}, NR^{b}SO₂NR^{b}R^{c}, N═S(═O)ₚR^{c}R^{c}, S(═O)ₒ(═NR^{b})_{q}R^{c}, SF₅, OCN, SCN, Si(R^{d})₃ or a three- to six-membered saturated, or partially unsaturated or aromatic carbo- or heterocyclic ring, which may contain 1 to 3 heteroatoms selected from N—(R^{c})ₚ, O, and S which may be oxidized, and wherein the aforementioned groups and the carbo- or heterocyclic ring may be partially or fully substituted with R^{aa}, or
   two geminally bound groups R^{a} together may form a group selected from ═O, ═S, ═CR^{b}R^{c}, ═NR^{c}, ═NOR^{c}, and ═NNR^{c}R^{c};
R^{aa} is each independently halogen, C₁-C₆-alkyl, C₁-C₆-haloalkyl, C₁-C₆-alkoxy or C₁-C₆-haloalkoxy;
R^{b} is each independently hydrogen, C₁-C₆-alkyl, C₁-C₆-haloalkyl, C₁-C₆-alkoxy, C₁-C₆-haloalkoxy or a three- to six-membered saturated, or partially unsaturated or aromatic carbo- or heterocyclic ring, which may contain 1 to 3 heteroatoms selected from N(R^{c})ₚ, O, and S, wherein S may be oxidized and which carbo- or heterocyclic ring may be partially or fully substituted with R^{aa};
R^{c} is each independently hydrogen, C₁-C₄-alkyl, C₁-C₄-haloalkyl, C₁-C₄-alkylcarbonyl, C₁-C₆ cycloalkyl, or a three- to six-membered saturated, partially unsaturated or aromatic carbo- or heterocyclic ring, which may contain 1 to 3 heteroatoms selected from N(R^{aa})ₚ, O and S, wherein S may be oxidized and wherein the carbo- or heterocyclic ring may be partially or fully substituted with R^{aa};
wherein two geminally bound groups R^{b}R^{b}, R^{c}R^{b} or R^{c}R^{c} together with the atom to which they are bound, may form a 3-, 4-, 5-, 6- or 7-membered saturated, partially unsaturated or aromatic carbo- or heterocyclic ring, which may contain 1 to 2 heteroatoms or heteroatoms groups selected from N, O, S, NO, SO and SO₂ and wherein the carbo- or heterocyclic ring may be partially or fully substituted with R^{a};
R^{d} is each independently hydrogen, phenyl, C₁-C₆-alkyl, C₂-C₆-alkenyl, C₂-C₆-alkynyl, C₃-C₈-cycloalkyl, or C₁-C₆-alkoxyalkyl, wherein the above mentioned groups may be substituted with one or more halogen;
R^{e} is each independently C₁-C₄-alkyl, C₁-C₄-haloalkyl, C₁-C₄-alkylcarbonyl, C₁-C₆ cycloalkyl, or a three- to six-membered saturated, partially unsaturated or aromatic carbo- or heterocyclic ring, which may contain 1 to 3 heteroatoms selected from N(R^{aa})ₚ, O and S, wherein S may be oxidized and wherein the carbo- or heterocyclic ring may be partially or fully substituted with R^{aa};
m is 0, 1, or 2;
n is 0, 1 or 2;
p is 0 or 1;
R⁵ is hydrogen, C₁-C₆-alkyl, C₂-C₆-alkenyl, C₂-C₆-alkynyl, which groups may be independently from each other substituted with one or up to five halogen or with one NO₂, CN, C₃-C₆-cycloalkyl, O—R⁵¹, —S(O)_{q}—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl,
   wherein aryl, hetaryl, heterocyclyl or oxoheterocyclyl may in turn be substituted with one or up to three halogen, NO₂, CN, C₁-C₆-alkyl, C₆-C₆-cycloalkyl, C₁-C₆-haloalkyl, C₁-C₆-alkoxy, C₁-C₆-haloalkoxy, C₁-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, C₁-C₆-alkylcarbonylamino, aryl or hetaryl;
   wherein aryl and hetaryl may be substituted with one or more, identical or different, halogen, CN, NO₂, hydroxy, C₁-C₆-alkyl, C₂-C₆-alkenyl, C₁-C₆-alkynyl, C₁-C₆-alkoxy, C₁-C₆-haloalkyl, C₁-C₆-haloalkoxy or C₁-C₆-alkylthio;
   or
R⁵ is C₃-C₆-cycloalkyl, C₃-C₆-heterocyclyl, which groups may be independently from each other substituted with one to three halogen or one CN, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulfinyl, C₁-C₆-alkylsulfonyl, aryl, hetaryl, aryl-C₁-C₆-alkyl or hetaryl-C₁-C₆-alkyl, wherein aryl, hetaryl, aryl-C₁-C₆-alkyl and hetaryl-C₁-C₆-alkyl may be substituted with one to three halogen, CN, NO₂, C₁-C₄-alkyl, C₁-C₄-haloalkyl, C₁-C₄-alkoxy, C₁-C₄-haloalkoxy;
   or
R⁵ is aryl or hetaryl, which groups may be independently from each other substituted with one to three halogen, NO₂, amino, CN, SF₅, SCN, C₁-C₆-alkylamino, di-(C₁-C₆)-alkylamino, hydroxy, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, or tri-(C₁-C₆-alkyl)-silyl;
q is 0, 1 or 2;
R⁵¹ is hydrogen, C₁-C₆-alkyl, C₂-C₆-alkenyl, C₂-C₆-alkynyl, C₃-C₆-cycloalkyl or C₃-C₆-heterocyclyl, which groups may be independently from each other substituted with one to three halogen or one NO₂, CN, C₃-C₆-cycloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-alkylcarbonyl, C₁-C₆-alkoxyimino-C₁-C₆-alkyl, C₃-C₆-cycloalkyl carbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, aryl or hetaryl, wherein aryl and hetaryl may be substituted with one to three halogen, CN, NO₂, C₁-C₄-alkyl, C₁-C₄-haloalkyl, C₁-C₄-alkoxy or C₁-C₄-haloalkoxy;
   or
R⁵¹ is aryl or hetaryl, which groups may be independently from each other substituted with one to three halogen, NO₂, amino, CN, SF₅, SCN, C₁-C₆-alkylamino, di-(C₁-C₆)-alkylamino, hydroxy, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₁-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, or tri-(C₁-C₆-alkyl)-silyl;
R⁵² is C₁-C₆-alkyl, C₂-C₆-alkenyl, C₂-C₆-alkynyl, C₃-C₆-cycloalkyl or C₃-C₆-heterocyclyl, which groups may be independently from each other substituted with one to three halogen, NO₂, CN, C₃-C₆-cycloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-alkylcarbonyl, C₁-C₆-alkoxyimino-C₁-C₆-alkyl, C₃-C₆-cycloalkylcarbonyl, C₁-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, aryl or hetaryl, wherein aryl and hetaryl may be substituted with one to three halogen, CN, NO₂, C₁-C₄-alkyl, C₁-C₄-haloalkyl, C₁-C₄-alkoxy or C₁-C₄-haloalkoxy;
   or
R⁵² is aryl or hetaryl, which groups may be independently from each other substituted with one to three halogen, NO₂, amino, CN, SF₅, SCN, C₁-C₆-alkylamino, di-(C₁-C₆)-alkylamino, hydroxy, COOH, C₁-C₆-alkyl, C₁-C₆-cycloalkyl, C₁-C₆-haloalkyl, C₁-C₆-alkoxy, C₁-C₆-haloalkoxy, C₃-C₆-halocycloalkyl, C₁-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₁-C₆-cycloalkyl-C₁-C₆-alkyl, C₂-C₆-alkenyl, C₁-C₆-alkynyl, C₁-C₆-alkylthio, C₁-C₆-alkylcarbonyl, C₁-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, or tri-(C₁-C₆-alkyl)-silyl;
R⁵³ is hydrogen, C₁-C₆-alkyl, C₂-C₆-alkenyl, C₂-C₆-alkynyl, C₃-C₆-cycloalkyl, C₁-C₆-alkoxy, C₁-C₆-alkoxy-C₁-C₆-alkyl, C₁-C₆-alkoxycarbonyl, C₁-C₆-alkylcarbonyl, aryl, hetaryl, arylcarbonyl or hetarylcarbonyl, wherein aryl and hetaryl may be substituted with one to three halogen, CN, NO₂, C₁-C₄-alkyl, C₁-C₄-haloalkyl, C₁-C₄-alkoxy or C₁-C₄-haloalkoxy;
R⁵⁴ is hydrogen, C₁-C₆-alkyl, C₂-C₆-alkenyl, C₂-C₆-alkynyl, C₃-C₆-cycloalkyl or C₃-C₆-cycloalkyl-C₁-C₆-alkyl, which groups may be independently from each other substituted with one to five halogen or one CN, NO₂, hydroxy, C₁-C₆-alkyl, C₃-C₆-cycloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-alkoxycarbonyl, C₁-C₆-alkylcarbonyl or tri-(C₁-C₆-alkyl)-silyl;
   or
R⁵⁴ is aryl, hetaryl, aryl-C₁-C₆-alkyl or hetaryl-C₁-C₆-alkyl, which groups may be independently from each other substituted with halogen, CN, NO₂, hydroxy, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₂-C₆-alkoxycarbonyl or C₂-C₆-alkylcarbonyl,
   or
R⁵³ and R⁵⁴ are connected through two to six carbon atoms and form a ring, which may comprise an additional atom selected from O, S or N, and which may be substituted with one to four C₁-C₂-alkyl, halogen, CN, amino or C₁-C₂-alkoxy;
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, wherein aryl, hetaryl, aryl-C₁-C₆-alkyl and hetaryl-C₁-C₆-alkyl, may be substituted with one or more, identical or different, halogen, CN, NO₂, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl;
R⁵⁶ is C₁-C₆-alkyl, C₃-C₆-cycloalkyl, C₃-C₆-halocycloalkyl, C₁-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, wherein aryl, hetaryl and aryl-C₁-C₆-alkyl may be substituted with one or more, identical or different, halogen, CN, NO₂, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl;
R⁶ is C₁-C₆-alkyl, C₃-C₆-cycloalkyl, C₁-C₆-haloalkyl, C₁-C₆-halocycloalkyl, C₁-C₆-cycloalkyl-C₁-C₆-alkyl, C₁-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, wherein aryl, hetaryl, aryl-C₁-C₆-alkyl and hetaryl-C₁-C₆-alkyl, may be substituted with one or more, identical or different halogen, CN, NO₂, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl;
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl;
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl, C₁-C₆-alkylcarbonyl;
R⁸ is hydrogen, C₁-C₆-alkyl, C₂-C₆-alkenyl, C₂-C₆-alkynyl, which groups may be independently from each other substituted with one to five halogen or one CN, NO₂, hydroxy, C₁-C₆-alkyl, C₃-C₆-cycloalkyl, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₁-C₄-alkylsulfimino-C₁-C₄-alkyl, C₁-C₄-alkylsulfimino-C₂-C₅-alkylcarbonyl, C₁-C₄-alkylsulfoximino, C₁-C₄-alkylsulfoximino-C₁-C₄-alkyl, C₁-C₄-alkylsulfoximino-C₂-C₅-alkylcarbonyl, C₁-C₆-alkoxycarbonyl, C₁-C₆-alkylcarbonyl or C₃-C₆-trialkylsilyl;
   or
R⁸ is aryl-C₁-C₆-alkyl or hetaryl-C₁-C₆-alkyl, C₃-C₁₂-cycloalkyl, C₃-C₁₂-cycloalkyl-C₁-C₆-alkyl or C₄-C₁₂-bicycloalkyl, which groups may be substituted with halogen, CN, NO₂, hydroxy, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₂-C₆-alkoxycarbonyl or C₂-C₆-alkylcarbonyl,
   or
R⁸ is a five- to six-membered aromatic or heteroaromatic ring which may be substituted with one or more identical or different substituents, a four- to six-membered partially saturated ring, a saturated heterocyclic ring, or a saturated or aromatic heterobicyclic ring which may comprise one to three heteroatoms from O, S or N and which may be substituted with one or more substituents, wherein the substituents are independently from each other halogen, CN, NO₂, hydroxy, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₂-C₆-alkoxycarbonyl or C₂-C₆-alkylcarbonyl,
   or
R⁷ and R⁸ are connected through two to six carbon atoms and form a ring, which may comprise an additional atom selected from O, S or N, and which may be substituted with one to four C₁-C₂-alkyl, halogen, CN, amino or C₁-C₂-alkoxy;
or a stereoisomer, tautomer, salt, or N-oxide thereof.

WO2017/093214 describes certain mesoionic imidazolium compounds.

WO2014/167084 describes certain substituted pyrimidinium compounds with heterocyclic substituents for combating invertebrate pests.

The substituted imidazolium compounds of formula (I) according to the present invention, with their characteristic core and substitution pattern, have not yet been described for pesticidal uses or pesticidal applications in agricultural industry or veterinary practice.

The substituted compounds of the formula (I), and their agriculturally acceptable salts are highly active against animal pest, i.e. harmful arthropodes and nematodes, especially against insects and acaridae which are difficult to control by other means.

Moreover, the present invention relates to and includes the following embodiments:
compositions comprising at least one compound of formula (I) as defined above;
agricultural and veterinary compositions comprising an amount of at least one compound of formula (I) or an enantiomer, diasteromer or salt thereof as defined above;
a method for combating invertebrate pests, infestation, or infection by invertebrate pests, which method comprises contacting said pest or its food supply, habitat or breeding grounds with a pesticidally effective amount of at least one compound of formula (I) as defined above or a composition thereof;
a method for controlling invertebrate pests, infestation, or infection by invertebrate pests, which method comprises contacting said pest or its food supply, habitat or breeding grounds with a pesticidally effective amount of at least one compound of formula (I) as defined above or a composition comprising at least one compound of formula (I);
a method for preventing or protecting against invertebrate pests comprising contacting the invertebrate pests, or their food supply, habitat or breeding grounds with substituted imidazolium compounds of the general formula (I) as defined above or a composition comprising at least one compound of formula (I) as defined above or a composition comprising at least one compound of formula (I);
a method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);
a non-therapeutic method for treating animals infested or infected by parasites or preventing animals of getting infected or infested by parasites or protecting animals against infestation or infection by parasites which comprises orally, topically or parenterally administering or applying to the animals a parasiticidally effective amount of a compound of formula (I) as defined above or a composition comprising at least one compound of formula (I);
a method for treating, controlling, preventing or protecting animals against infestation or infection by parasites by administering or applying orally, topically or parenterally to the animals a substituted imidazolium compound of the general formula (I) as defined above or a composition comprising at least one compound of formula (I);
seed comprising a compound of formula (I) as defined above, in an amount of from 0.1 g to 10 kg per 100 kg of seed;
the use of the compounds of formula (I) as defined above for protecting growing plants or plant propagation material from attack or infestation by invertebrate pests;
the use of compounds of formula (I) or the enantiomers, diastereomers or veterinary acceptable salts thereof for combating parasites in and on animals;
a process for the preparation of a veterinary composition for treating, controlling, preventing or protecting animals against infestation or infection by parasites which comprises adding a parasiticidally effective amount of an compound of formula (I) or the enantiomers, diastereomers and/or veterinary acceptable salt thereof to a carrier composition suitable for veterinary use;
the use of a compound of formula (I) or the enantiomers, diastereomers and/or veterinary acceptable salt thereof for the preparation of a medicament for treating, controlling, preventing or protecting animals against infestation or infection by parasites.

All the compounds of the present invention including if applicable their stereoisomers, their tautomers, their salts or their N-oxides as well as compositions thereof are particularly useful for controlling invertebrate pests, in particular for controlling arthropods and nematodes and especially insects. Therefore, the invention relates to the use of a compound as disclosed in the present invention, for combating or controlling invertebrate pests, in particular invertebrate pests of the group of insects, arachnids or nematodes.

The term “compound(s) according to the invention” or “compound(s) of formula (I)” as used in the present invention refers to and comprises the compound(s) as defined herein and/or stereoisomers), salt(s), tautomer(s) or N-oxide(s) thereof. The term “compound(s) of the present invention” is to be understood as equivalent to the term “compound(s) according to the invention”, therefore also comprising stereoisomer(s), salt(s), tautomer(s) or N-oxide(s) of compounds of formula (I).

The term “composition(s) according to the invention” or “composition(s) of the present invention” encompasses composition(s) comprising at least one compound of formula (I) according to the invention as defined above, therefore also including a stereoisomer, an agriculturally or veterinary acceptable salt, tautomer or an N-oxide of the compounds of formula (I).

The compounds of the formula (I) are present in mesomeric forms.

These forms may be expressed in different isoelectronic formulae, each having the formal positive and negative charges on different atoms (as shown below). The present invention extends to all representative isoelectronic structures of compounds of formula I.

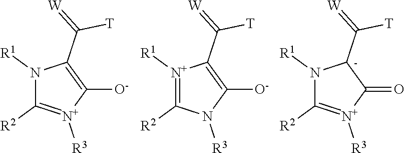

The compounds of the formula (I) may have one or more centers of chirality, i.e. they are present as mixtures of enantiomers or diastereomers. The invention provides both the single pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond or amide group. The term “stereoisomers)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula (I), i.e. to single enantiomers or diastereomers, as well as to mixtures thereof.

The compounds of the present invention may be amorphous or may exist in one or more different crystalline states (polymorphs) or modifications which may have a different macroscopic properties such as stability or show different biological properties such as activities. The present invention includes both amorphous and crystalline compounds of the formula (I), mixtures of different crystalline states or modifications of the respective compound I, as well as amorphous or crystalline salts thereof.

Salts of the compounds of the formula (I) are preferably agriculturally and/or veterinary acceptable salts. They can be formed in a customary method, e.g. by reacting the compound with an acid of the anion in question if the compound of formula (I) has a basic functionality or by reacting an acidic compound of formula (I) with a suitable base.

Suitable agriculturally or veterinary useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an add of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The organic moieties groups mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “Cₙ-Cₘ-alkyl” as used herein (and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl) refers to a branched or unbranched saturated hydrocarbon group having n to m, e.g. 1 to 10 carbon atoms, preferably 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methyl pentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein (and also in Cₙ-Cₘ-haloalkylsulfinyl and Cₙ-Cₘ-haloalkylsulfonyl) refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₙ-Cₘ-alkoxy” and “Cₙ-Cₘ-alkylthio” (or Cₙ-Cₘ-alkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen (or sulfur linkages, respectively) at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “Cₙ-Cₘ-haloalkoxy” and “Cₙ-Cₘ-haloalkylthio” (or Cₙ-Cₘ-haloalkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-Cₘ-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term “Cₙ-Cₘ-alkoxy-Cₙ-Cₘ-alkyl” as used herein refers to alkyl having n to m carbon atoms, e.g. like specific examples mentioned above, wherein one hydrogen atom of the alkyl radical is replaced by an Cₙ-Cₘ-alkoxy group; wherein the value of n and m of the alkoxy group are independently chosen from that of the alkyl group.

The suffix “-carbonyl” in a group or “C(═O)” denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxy carbonyl.

The term “aryl” as used herein refers to a mono-, bi- or tricyclic aromatic hydrocarbon radical such as phenyl or naphthyl, in particular phenyl (also referred as to C₆H₅ as substituent).

The term “ring system” denotes two or more directly connected rings.

The term “C₃-Cₘ-cycloalkyl” as used herein refers to a monocyclic ring of 3- to m-membered saturated cycloaliphatic radicals, e.g. cyclopropyl, cyclobutyl, cyclopentyl, cyclohexyl, cycloheptyl, cyclooctyl and cyclodecyl.

The term “alkylcycloalkyl” denotes as well as the term “alkyl which may be substituted with cycloalkyl” an alkyl group which is substituted with a cycloalkyl ring, wherein alkyl and cycloakyl are as herein defined.

The term “cycloalkylalkyl” denotes as well as the term “cycloalkyl which may be substituted with alkyl” a cycloalkyl ring which is substituted with an alkyl group, wherein alkyl and cycloakyl are as herein defined.

The term “alkylcycloalkylalkyl” denotes as well as the term “alkylcycloalkyl which may be substituted with alkyl” an alkylcycloalkyl group which is substituted with an alkyl, wherein alkyl and alkylcycloakyl are as herein defined.

The term “C₃-Cₘ-cycloalkenyl” as used herein refers to a monocyclic ring of 3- to m-membered partially unsaturated cycloaliphatic radicals.

The term “cycloalkylcycloalkyl” denotes as well as the term “cycloalkyl which may be substituted with cycloalkyl” a cycloalkyl substitution on another cycloalkyl ring, wherein each cycloalkyl ring independently has from 3 to 7 carbon atom ring members and the cycloalkyls are linked through one single bond or have one common carbon atom. Examples of cycloalkylcycloalkyl include cyclopropylcyclopropyl (e.g. 1,1′-bicyclopropyl-2-yl), cyclohexylcyclohexyl wherein the two rings are linked through one single common carbon atom (e.g. 1,T-bicyclohexyl-2-yl), cyclohexylcyclopentyl wherein the two rings are linked through one single bond (e.g. 4-cyclopentylcyclohexyl) and their different stereoisomers such as (1R,2S)-1,1′-bicyclopropyl-2-yl and (1R,2R)-1,1′-bicyclopropyl-2-yl.

The term “3- to 6-membered carbocyclic ring” as used herein refers to cyclopropane, cyclobutane, cyclopentane and cyclohexane rings.

The term “3-, 4-, 5-, 6- or 7-membered saturated, partially unsaturated or aromatic heterocyclic ring which may contain 1, 2, 3 or 4 heteroatoms” or “containing heteroatom groups”, wherein those heteroatom(s) (group(s)) are selected from N (N-substituted groups), O and S (S-substituted groups) as used herein refers to monocydic radicals, the monocyclic radicals being saturated, partially unsaturated or aromatic (completely unsaturated). The heterocydic radical may be attached to the remainder of the molecule via a carbon ring member or via a nitrogen ring member.

Examples of 3-, 4-, 5-, 6- or 7-membered saturated heterocyclyl or heterocyclic rings include: oxiranyl, aziridinyl, azetidinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin 5 yl, 1,2,4-thiadiazolidin-3-yl, 1,2,4-thiadiazolidin-5-yl, 1,2,4-triazolidin-3-yl,-1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl or heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin 3 yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-li, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7 tetrahydro[1H]azepin-1-, -2-, -3-, -4-, -5-, -6- or -7-yl, 2,3,6,7 tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7 tetrahydro[1H]oxepin-2-, -3-, -4-, -5-, -6- or -7-yl, 2,3,6,7 tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

Examples of 5- or 6-membered aromatic heterocyclic (hetaryl) or heteroaromatic rings are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A “C₂-Cₘ-alkylene” is divalent branched or preferably unbranched saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

Embodiments and preferred compounds of the present invention for use in pesticidal methods and for insecticidal application purposes are outlined in the following paragraphs.

The remarks made below concerning preferred embodiments of the variables (substituents) of the compounds according to the invention, especially with respect to their substituents W, T, R¹, R², R³, R⁴, Het, R^{4a}, R^{a}, R^{aa}, R^{b}, R^{c}, R^{d}, R^{e}, m, n, p, R⁵, R⁵¹, R⁵², R⁵³, R⁵⁴, R⁵⁵, R⁶, R⁷, R^{7a} and R⁸ are valid both on their own and, in particular, in every possible combination with each other and where applicable, the uses, the methods and the compositions according to the invention.

In a particular embodiment, the variables of the compounds of formula (I) have the following meanings, these meanings, both on their own and in combination with one another, being particular embodiments of the compounds of the formula (I):

In one embodiment <W-1> of the invention, in the compounds of formula (I), W is O, and T is R⁵. In a further embodiment <W-2> of the invention, W is O, and T is C₁-C₆-alkyl, C₂-C₆-alkenyl, C₂-C₆-alkynyl, each optionally substituted with one or up to five halogen or with one NO₂, CN, C₃-C₆-cycloalkyl, O—R⁵¹, —S(O)_{q}—R⁵², —N(R⁵³)(R⁵⁴).

In a further embodiment <W-3>, W is O and T is methyl, optionally substituted with halogen, cyano, C₁-C₆-alkoxy, C₁-C₆-alkylthio, C₁-C₆-alkylsulfinyl, C₁-C₆-alkylsulfonyl, C₃-C₆-cycloalkyl.

In a further embodiment <W-4>, W is O and T is OR⁶, wherein R⁶ is C₁-C₆-alkyl, C₃-C₆-cycloalkyl, C₁-C₆-haloalkyl, optionally substituted with halogen, cyano, C₁-C₆-alkoxy, C₁-C₆-alkylthio, C₁-C₆-alkylsulfinyl, C₁-C₆-alkylsulfonyl, C₃-C₆-cycloalkyl.

In an embodiment <1-1>, R¹ is NO₂, CN, C₁-C₈-alkyl, C₂-C₆-alkenyl, C₂-C₈-alkynyl, C₁-C₆-alkoxy, C₃-C₆-cycloalkyl, C₄-C₁₀-cycloalkenyl or C₅-C₁₁-cycloalkylcycloalkyl, wherein the C-atoms of the aforementioned groups may be unsubstituted, or partially or fully substituted with R^{a}, wherein R^{a} has the meaning as hereunder described.

In another embodiment <1-2>, R¹ is a three- to ten-membered saturated, or partially saturated or heterocyclic ring system, which may contain 1 to 3 heteroatoms selected from N(R^{c})ₚ, O, and S, wherein S may be oxidized and which heterocyclic ring may be unsubstituted or substituted with R^{a}.

In a further embodiment <1-3>, R¹ is C₁-C₄-alkyl, C₂-C₈-alkenyl, C₁-C₆-alkoxy, C₃-C₆-cycloalkyl or C₅-C₁₁-cycloalkylcycloalkyl, wherein the C-atoms of the aforementioned groups may be unsubstituted, or partially or fully substituted with halogen.

In a further embodiment <1-4>, R¹ is C₁-C₄-alkyl, C₂-C₈-alkenyl, C₃-C₆-cycloalkyl, phenyl or benzyl, wherein the C-atoms of the aforementioned groups may be unsubstituted, or partially or fully substituted with R^{a}, wherein R^{a} has the meaning as hereunder described.

In a further embodiment <1-5>, R¹ is C₁-C₄-alkyl, C₃-C₆-cycloalkyl or phenyl, wherein the C-atoms of the aforementioned groups may be unsubstituted, or partially or fully substituted with halogen or C₁-C₄-alkyl.

In a further embodiment <1-6>, R¹ is C₁-C₄-alkyl, C₂-C₄-alkenyl, phenyl or benzyl, wherein the c-atoms of the aforementioned groups may be partially or fully substituted with halogen, preferably Cl or F.

In a further embodiment <1-7>, R¹ is C₁-C₄-alkyl, C₃-C₆-cycloalkyl or phenyl, preferably CH₃, CH₂CH₃, CH(CH₃)₂, cyclopropyl or phenyl.

In a further embodiment <1-8>, R¹ is C₁-C₃-alkyl, preferably CH₃, CH₂CH₃ or CH(CH₃)₂, particularly R¹ is CH₃, particularly R¹ is CH₂CH₃.

In a further embodiment <1-9>, R¹ is C₁-C₄-alkyl, C₃-C₆-cycloalkyl, C₂-C₄-alkenyl, benzyl or phenyl, which groups may be partially or fully substituted with halogen or C₁-C₄-alkyl.

In an embodiment <2-1>, R² and R³ are each independently selected from C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with R^{a}. In an embodiment <2-1a>, R² and R³ are each independently selected from C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with R^{a}; OR^{c}, OC(═O)R^{c}, NR^{b}R^{c}, S(O)ₘR^{b}. In an embodiment <2-1 b>, R² and R³ are each independently selected from C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with halogen.

In an embodiment <2-1 c>, R² and R³ are each independently selected from C₁-C₈-alkyl, C₃-C₁₀-cycloalkyl, which groups may be unsubstituted, partially or fully substituted with halogen.

In a further embodiment <2-2>, R² and R³ are each independently selected from C(═O)R^{b}, C(═O)OR^{e}, NR^{b}R^{c}, C(═O)NR^{b}R^{c}, C(═S)NR^{b}R^{c}; C(═NR^{c})R^{c}, C(═NOR^{c})R^{c}—. C(═NNR^{b}R^{c})R^{c}, C(═NN(C(═O)R^{b})R^{c})R^{c}, C(═NN(C═O)OR^{c})(R^{c})₂.

In a further embodiment <2-3>, R² and R³ are each independently selected from OR^{c}, OC(═O)R^{c}, OC(═O)OR^{e}, OC(═O)NR^{b}R^{e}.

In a further embodiment <2-4>, R² and R³ are each independently selected from NR^{b}R^{c}, N(R^{c})C(═O)R^{c}, N(R^{c})C(═O)OR^{e}, N(R^{c})C(═O)NR^{b}R^{c}, NR^{c}SO₂R^{b}, NR^{c}SO₂NR^{b}R^{c}, N═CR^{b}R^{c}; In a further embodiment <2-5>, R² and R³ are each independently selected from NR^{b}R^{c}, N(R^{c})C(═O)R^{c}, N(R^{c})C(═O)OR^{e}.

In a further embodiment <2-6>, R² and R³ are each independently selected from S(O)ₘR^{b}, SO₂NR^{b}R^{c}, S(═O)ₒ(═NR^{b})_{q}R^{c}.

In a further embodiment <2-7>, one of R² and R³ is R⁴. In a further embodiment <2-7a>, R³ is R⁴.

In a further embodiment <2-8>, R³ is substituted with one R⁴.

In a further embodiment <2-9>, R² and R³ form a four- to seven-membered ring, taken together with the carbon and nitrogen of the imidazole ring in formula (I), wherein each remaining ring member is selected from carbon atoms and up to 3 heteroatoms independently selected from up to 2 O, up to 2 S, and up to 3 N, wherein up to 2 carbon atom ring members are independently selected from C(═O) and C(═S), and the sulfur atom ring members are independently selected from S(═O)ₘ, wherein each ring member may be substituted with R^{a} and/or R^{c}; wherein the ring is substituted with one R⁴.

In a further embodiment <2-10>, R² and R³, together with the nitrogen and the carbon atom of the imidazolium ring, form a five or six membered ring, wherein each remaining ring member is selected from carbon atoms and up to one heteroatoms independently selected from O, S, and N(R^{c})ₚ, wherein each ring may be substituted with up to one R^{a}, wherein R^{a} has the meaning as hereunder described.

In a further embodiment <2-11>, R² and R³, together with the nitrogen and the carbon atom of the imidazolium ring, form a five membered ring, wherein each remaining ring member is selected from carbon atoms and up to one heteroatoms independently selected from O, S, and N(R^{c})ₚ.

In a further embodiment <2-12>, R² and R³, together with the nitrogen and the carbon atom of the imidazolium ring, form a six membered ring, wherein each remaining ring member is selected from carbon atoms and up to one heteroatoms independently selected from O, S, and N(R^{c})ₚ.

In a further embodiment <2-13>, R² and R³, together with the nitrogen and the carbon atom of the imidazolium ring, form a five or six membered ring resulting in the compounds of formula (II) selected from the group of compounds of formulae II-1 to II-16:

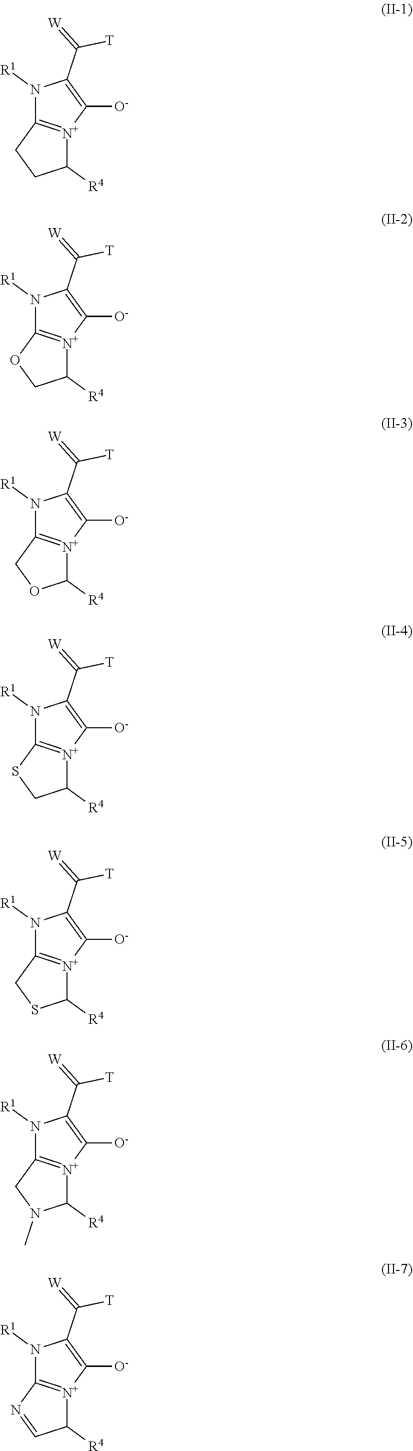

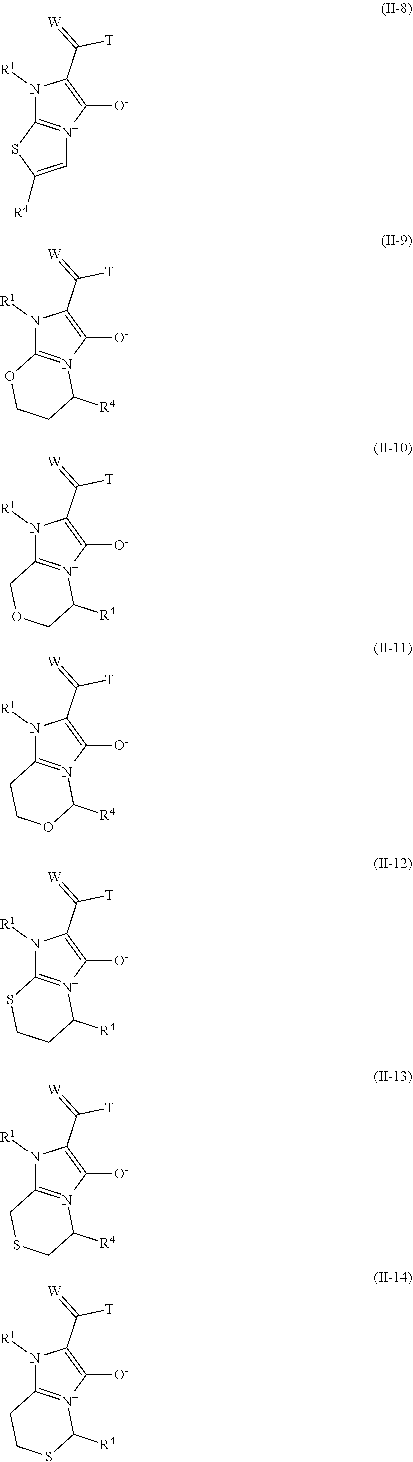

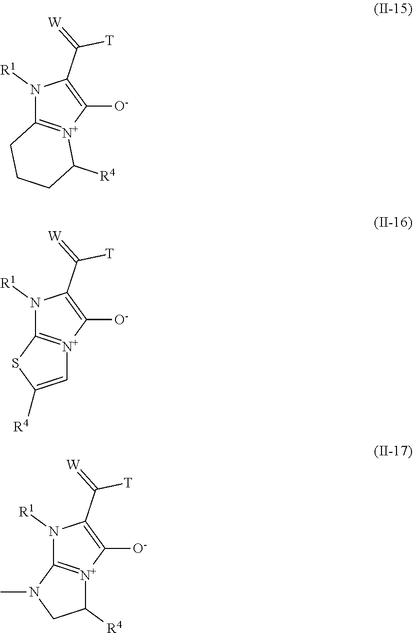

In a further embodiment, compounds of formula (I) are selected from the group of compounds of formulae II-2, II-4, II-9, II-12.

In an embodiment <4-1>, R⁴ is Het, and Het is selected from any one of the following ring systems D-1 to D-56:

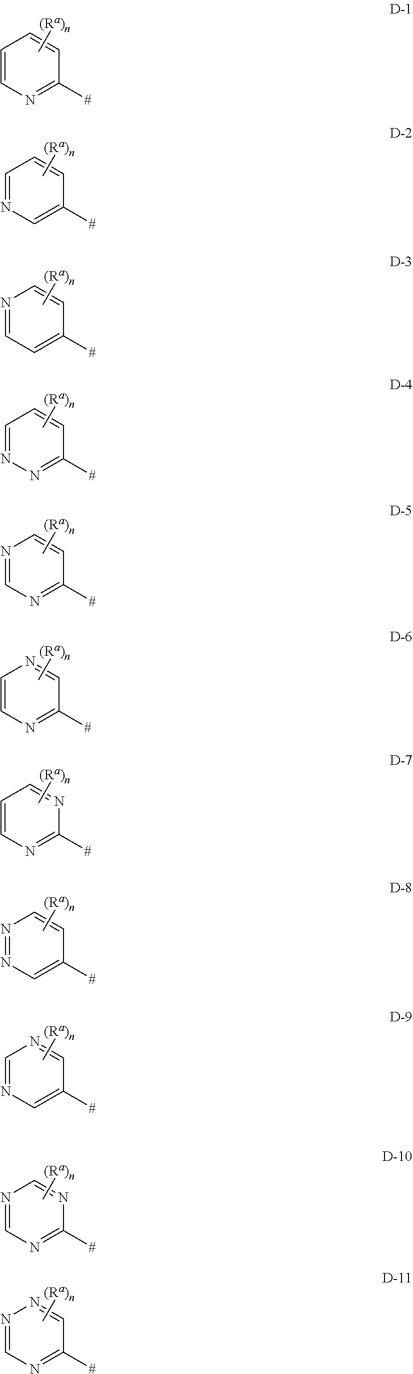

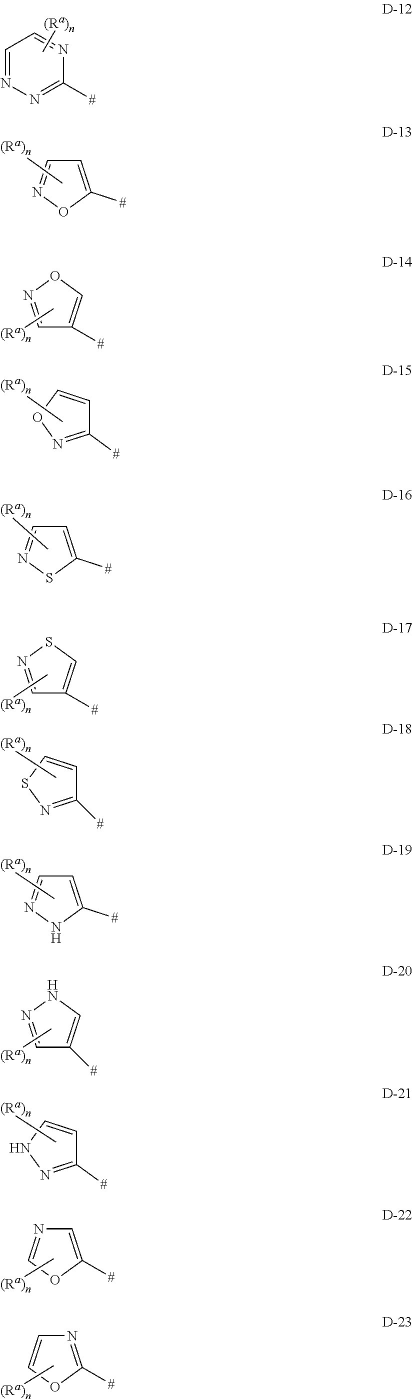

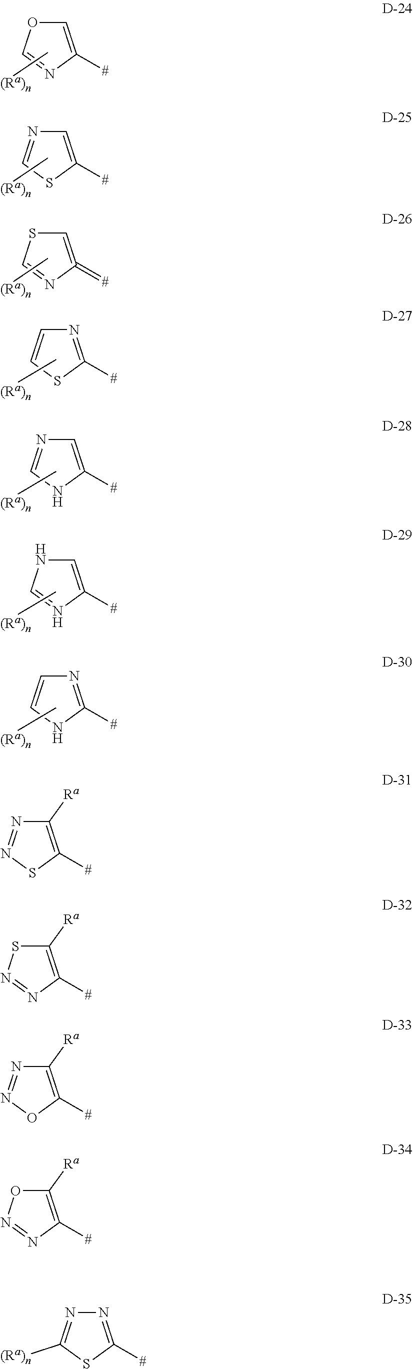

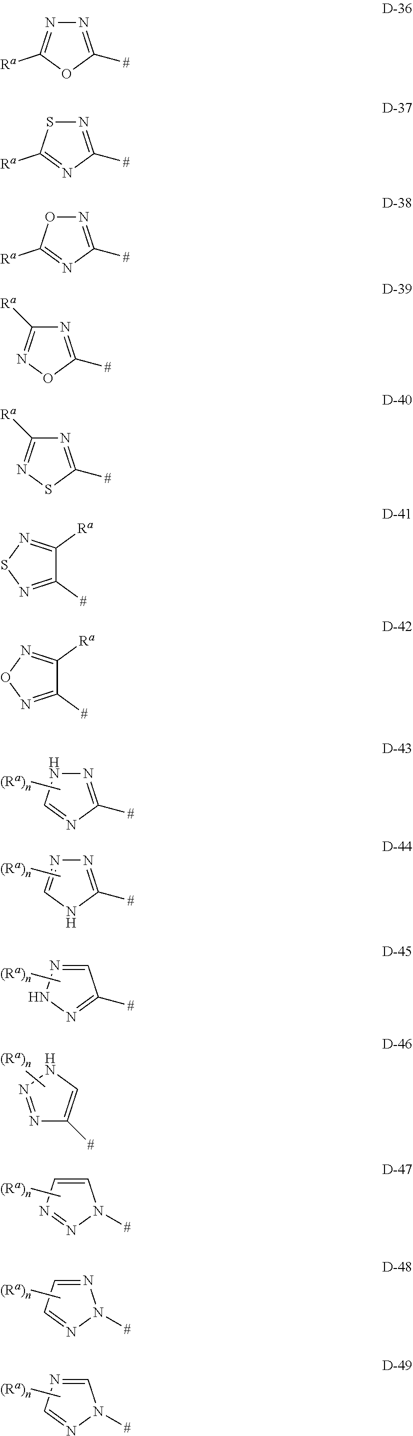

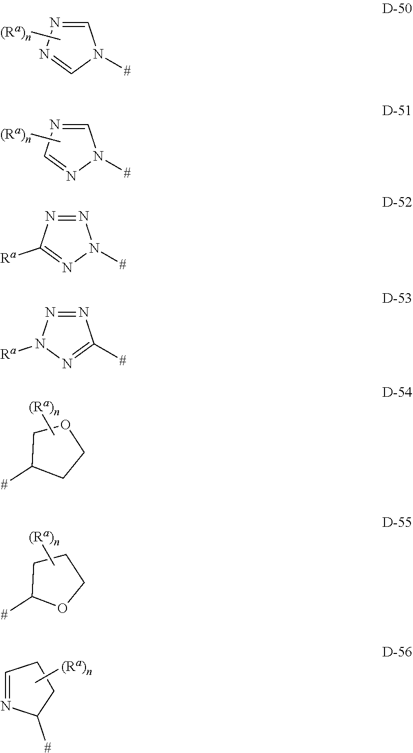

Wherever used in a structure, the following: #denotes the bond to A in formula (I)

In a further embodiment Het is selected from any one of the following ring systems:

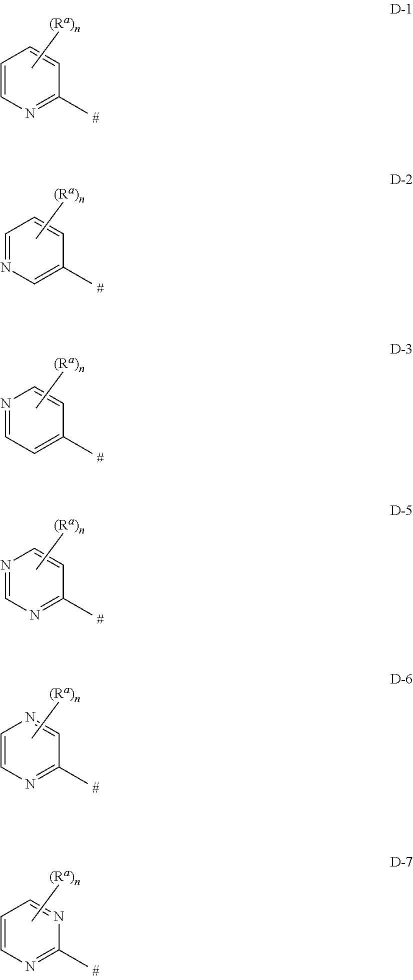

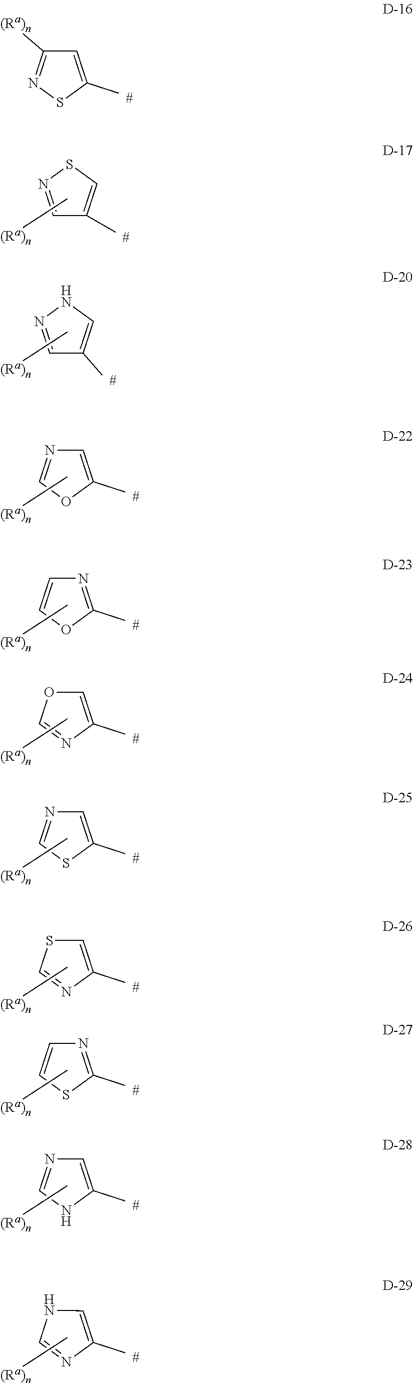

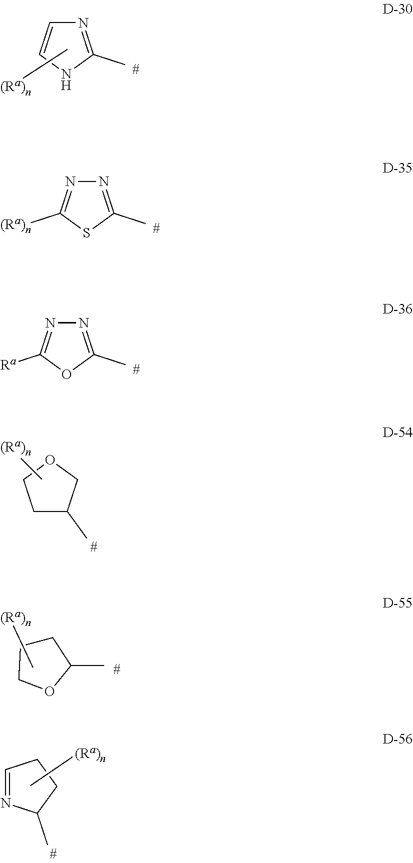

In a further embodiment <4-2>, Het is selected from the following rings systems D-2, D-9, D-22, D-25, D-28, D-29 and D-54:

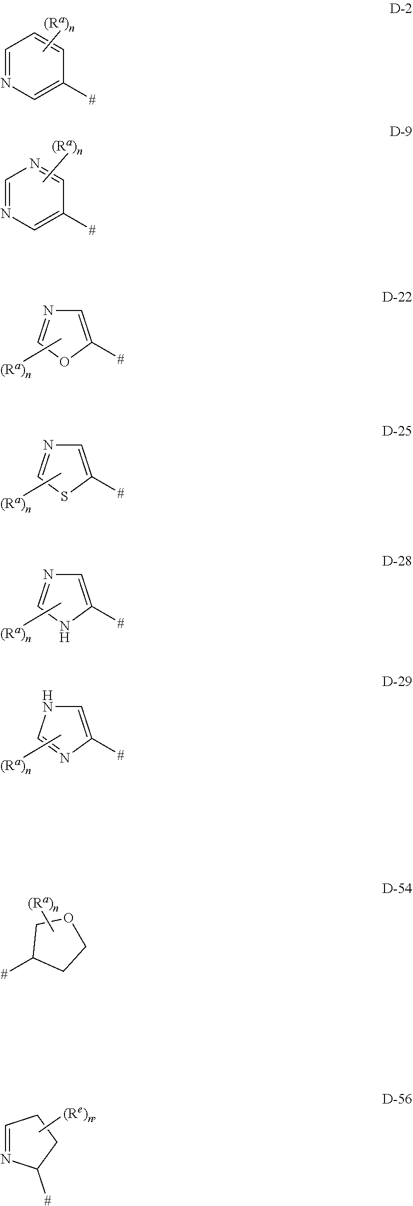

wherein R^{a} is halogen, C₁-C₄-haloalkyl, C₁-C₄-alkoxy or C₁-C₄-alkylthio or phenyl; preferably R^{a} is halogen or halomethyl.

In a further embodiment <4-3>, Het is selected from the following rings systems D-2, D-9, D-25 and D-56:

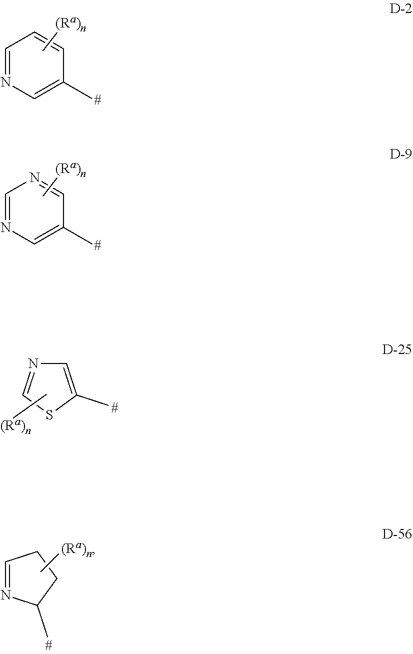

wherein R^{a} is halogen, C₁-C₄-haloalkyl, C₁-C₄-alkoxy or C₁-C₄-alkylthio or phenyl, preferably halogen or C₁-C₄-haloalkyl; more preferably R^{a} is Cl, Br, F or CF₃, most preferably R^{a} is Cl or CF₃.

In a further embodiment <4-4>, Het is selected from the following rings systems D-2, D-25 or D-54:

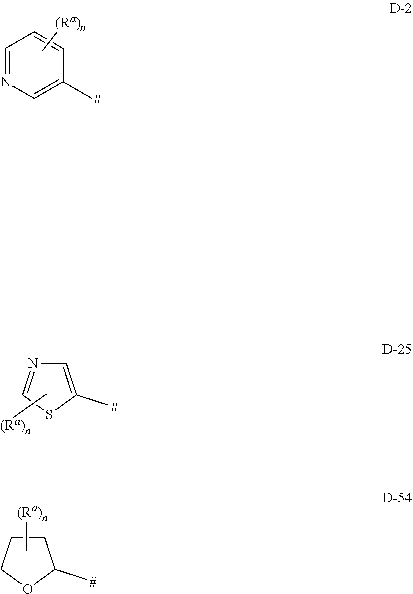

wherein R^{a} is halogen or C₁-C₄-haloalkyl; preferably R^{a} is Cl, Br, F or CF₃, most preferably R^{a} is Cl or CF₃.

In another embodiment <4-5>, Het is selected from the following rings systems D-2a, D-2b, D-2c, D-25, D-25a, preferably D-25a substituted with Cl, and D-56a:

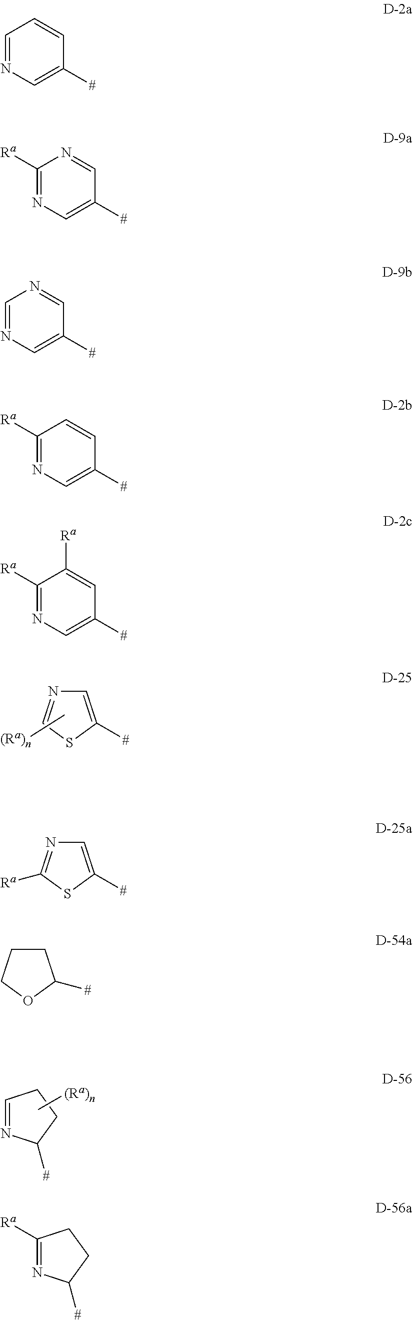

wherein R^{a} are independently from each other selected from Cl, Br, F and CF₃.

In another embodiment <4-6>, Het is D-2, preferably D-2b or D-2c, particularly D-2b, wherein R^{a} is Cl or CF₃.

In another embodiment <4-7>, Het is selected from D-2a, D-25, preferably D-25a substituted with Cl, D-9, preferably D-9a or D9b, D-56, preferably D-56a.

In another embodiment, Het is D-2a.

In another embodiment, Het is D-25, preferably D-25a substituted with Cl.

In another embodiment, Het is D-9, preferably D-9a or D9b.

In another embodiment, Het is D-56, preferably D-56a.

In a further embodiment <4-8>, R⁴ is R^{4a}, which is C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₇-cycloalkyl, C₃-C₇-halocycloalkyl, C₄-C₈-alkylcycloalkyl, C₄-C₈-haloalkylcycloalkyl, C₄-C₈-cycloalkylalkyl, C₄-C₈-halocycloalkylalkyl, C₁-C₆-alkoxy, C₁-C₆-haloalkoxy, C₂-C₆-alkylcarbonyl, C₂-C₆-haloalkylcarbonyl, CN; each optionally substituted with one or more substituents selected from C₁-C₆-alkyl, C₁-C₆-haloalkyl, C₁-C₆-alkoxy, C₁-C₆-haloalkoxy, C₂-C₄-alkenyl, C₂-C₄-alkynyl, C₃-C₆-cycloalkyl, CN, OR^{c}, NR^{b}R^{c}, NO₂.

In a further embodiment <4-9>, R⁴ is C₁-C₄-alkyl, C₁-C₄-haloalkyl, each optionally substituted with one or more substituents selected from CN, NO₂.

In a further embodiment <4-10>, R⁴ is CH₂CH₂CN or CH₂CN, preferably CH₂CH₂CN.

In an embodiment, R^{a} is halogen, C₁-C₆-alkyl, C₁-C₆-haloalkyl, C₁-C₆-alkoxy, C₁-C₆-haloalkoxy, C₁-C₆-cycloalkyl, CN, OR^{c}, NR^{b}R^{c}, NO₂, phenyl, pyridyl, thiazyl, furanyl, pyrimidinyl or thienyl, wherein the C-atoms aforementioned which groups may be unsubstituted or substituted with one or more R^{aa}, wherein R^{aa} is as hereunder defined.

In a further embodiment, R^{a} is halogen, C₁-C₄-alkyl, C₁-C₄-haloalkyl or C₃-C₆-cycloalkyl.

In a further embodiment, R^{a} is halogen, C₁-C₄-alkyl, C₁-C₄-haloalkyl or C₃-C₆-cycloalkyl.

In a further embodiment, R^{a} is halogen.

In an embodiment, R^{a} is halogen, CN, NO₂, S(O)ₘR^{b}, C(O)R^{c}, C(O)OR^{c}, C(O)NR^{b}R^{c}, C(═S)NR^{b}R^{c}, C₁-C₆-alkyl, C₃-C₆-cycloalkyl, C₂-C₆-alkenyl, C₂-C₆-alkynyl, C₁-C₆-alkoxy, C₂-C₆-alkenyloxy or C₂-C₆-alkynyloxy, wherein the C-atoms of the aforementioned groups may be unsubstituted, partially or fully substituted with R^{a}, wherein is as hereunder defined.

In a further embodiment, R^{a} is halogen, CN, C₁-C₆-alkyl, C₁-C₆-cycloalkyl, C₂-C₆-alkenyl, C₂-C₆-alkynyl, C₁-C₆-alkoxy, C₂-C₆-alkenyloxy or C₂-C₆-alkynyloxy, which C-atoms of the aforementioned groups may be unsubstituted, partially or fully substituted with R^{aa}, wherein R^{aa} is as hereunder defined.

In a further embodiment, R^{a} is halogen, CN, C₁-C₆-alkyl, C₃-C₆-cycloalkyl, C₂-C₆-alkenyl, C₂-C₆-alkynyl, C₁-C₆-alkoxy, C₂-C₆-alkenyloxy or C₂-C₆-alkynyloxy, wherein the C-atoms of the aforementioned groups may be unsubstituted, partially or fully substituted with halogen.

In a further embodiment, R^{a} is halogen, C₁-C₆-haloalkyl or C₁-C₆-alkoxy.

In a further embodiment, R^{a} is halogen, CN or C₁-C₂-haloalkyl.

In a further embodiment, R^{a} is halogen or C₁-C₂-haloalkyl.

In an embodiment, R^{a} is halogen, preferably Br, Cl or F, particularly Cl.

In another embodiment, R^{a} is C₁-C₂-haloalkyl, preferably halomethyl such as CHF₂ or CF₃, particularly CF₃.

In an embodiment, two geminally bound groups R^{a} together may form a group selected from ═O, ═S, ═CR^{b}R^{c}, ═NR^{c}, ═NOR^{c}, and ═NNR^{c}R^{c};

In another embodiment, two geminally bound groups R^{a} together may form a group selected from ═CR^{b}R^{c}, ═NR^{c}, ═NOR^{c}, and ═NNR^{c}R^{c}; In another embodiment, two geminally bound groups R^{a} together may form a group selected from ═O, ═S and ═N(C₁-C₆-alkyl).

In another embodiment, two geminally bound groups R^{a} together may form a=N(C₁-C₆-alkyl) group.

In an embodiment, R^{b} is hydrogen, C₁-C₆-alkyl, C₁-C₆-haloalkyl, C₁-C₆-alkoxy, C₁-C₆-haloalkoxy, phenyl, pyridyl, thiazyl or thienyl, wherein the C-atoms of the aforementioned groups may be substituted with R^{aa}, wherein R^{aa} is as hereunder defined. In a further embodiment, R^{b} is hydrogen, C₁-C₆-alkyl, C₁-C₆-haloalkyl, C₁-C₆-alkoxy or C₁-C₆-haloalkoxy. In a further embodiment, R^{b} is hydrogen, C₁-C₆-alkyl or C₁-C₆-haloalkyl. In an embodiment, R^{b} is C₁-C₆-alkyl or C₁-C₆-haloalkyl. In an embodiment, R^{b} is H.

In an embodiment, R^{c} is hydrogen, C₁-C₄-alkyl, C₁-C₄-haloalkyl, C₁-C₄-alkylcarbonyl, C₁-C₆ cycloalkyl, phenyl, pyridyl, thiazyl or thienyl wherein the C-atoms of the aforementioned groups may be substituted with R^{aa}, wherein R^{aa} is as hereunder defined. In a further embodiment, R^{c} is hydrogen, C₁-C₄-alkyl, C₁-C₄-haloalkyl, C₁-C₄-alkylcarbonyl, or C₁-C₆-cycloalkyl. In an embodiment, R^{c} is hydrogen, C₁-C₆-alkyl or C₁-C₆-haloalkyl. In an embodiment, R^{c} is C₁-C₆-alkyl or C₁-C₆-haloalkyl. In an embodiment, R^{c} is H.

In an embodiment, two geminally bound groups R^{b}R^{b}, R^{c}R^{b} or R^{c}R^{c} together with the atom to which they are bound, may form a 3-, 4-, 5-, 6- or 7-membered saturated, partially unsaturated or aromatic carbo- or heterocyclic ring, which may contain 1 to 2 heteroatoms or heteroatoms groups selected from N, O, S, NO, SO and SO₂ and wherein the carbo- or heterocyclic ring may be partially or fully substituted with R^{a}.

In another embodiment, two geminally bound groups R^{b}R^{b}, R^{c}R^{b} or R^{c}R^{c} together with the atom to which they are bound, may form a 5- or 6-membered saturated, partially unsaturated or aromatic carbocyclic ring, which ring may be partially or fully substituted with R^{a}, and wherein R^{a} is as hereunder defined.

In another embodiment, two geminally bound groups R^{b}R^{b}, R^{c}R^{b} or R^{c}R^{c} together with the atom to which they are bound, may form a 5- or 6-membered saturated, partially unsaturated or aromatic heterocyclic ring, which may contain 1 to 2 heteroatoms or heteroatoms groups selected from N, O, S, NO, SO and SO₂, wherein the heterocyclic ring may be partially or fully substituted with R^{a}, and wherein R^{a} is as hereunder defined.

In an embodiment, R^{d} is hydrogen, phenyl, C₁-C₄-alkyl or C₂-C₆-alkenyl, wherein the aforementioned groups may be substituted with one or more halogen. In a further embodiment, R^{d} is C₁-C₄-alkyl or phenyl, which may be substituted with halogen. In another embodiment, R^{c} C₁-C₄-alkyl, preferably CH₃.

In an embodiment, R^{e} is C₁-C₄-alkyl, C₁-C₄-haloalkyl, C₁-C₄-alkylcarbonyl, C₁-C₈ cycloalkyl, phenyl, pyridyl, thiazyl or thienyl wherein the aforementioned groups may be substituted with R^{aa}, wherein R^{aa} is as hereunder defined. In a further embodiment, R^{e} is C₁-C₄-alkyl, C₁-C₄-haloalkyl, C₁-C₄-alkylcarbonyl, or C₁-C₆-cycloalkyl. In a further embodiment, R^{e} is C₁-C₄-alkyl or C₁-C₄-haloalkyl.

In an embodiment, R^{aa} is halogen, C₁-C₆-alkyl or C₁-C₆-haloalkyl. In another embodiment, R^{aa} is C₁-C₆-alkoxy or C₁-C₆-haloalkoxy. In an embodiment, R^{aa} is halogen.

In an embodiment, R^{2a} is halogen, C₁-C₆-haloalkyl, C₁-C₆-alkoxy, C₁-C₆-haloalkoxy, OR^{c}, C(═O)OR^{c}, C(═O)NR^{b}R^{c}, or phenyl, wherein the C-atoms of the aforementioned groups may be unsubstituted or substituted with one or more R^{2aa}, wherein R^{2aa} is as hereunder defined, particularly R^{2a} is halogen, C₁-C₆-alkoxy, or C₁-C₆-haloalkoxy.

In an embodiment, two geminally bound groups R^{2a} together may form a group selected from ═O, ═S and ═N(C₁-C₆-alkyl).

In an embodiment, R^{2a} is halogen, C₁-C₆-alkyl, C₁-C₆-haloalkyl, C₁-C₆-alkoxy, C₁-C₆-haloalkoxy, C₃-C₆-cycloalkyl, CN, OR^{c}, NR^{b}R^{c}, NO₂, phenyl, pyridyl, thiazyl, furanyl, pyrimidinyl or thienyl, wherein the C-atoms of the aforementioned groups may be unsubstituted or substituted with one or more R^{2aa}, wherein R^{2aa} is as hereunder defined.

In a further embodiment, R^{2a} is halogen, C₁-C₄-haloalkyl or C₃-C₆-haloalkoxy.

In a another embodiment, R^{2a} is phenyl which may be substituted with one or more R^{2aa}.

In a another embodiment, R^{2a} is halogen. In another embodiment, R^{2a} is C₁-C₆-haloalkyl. In another embodiment, R^{2a} is C₁-C₆-haloalkoxy.

In another embodiment, R^{2a} is halogen, CN, NO₂, S(O)ₘR^{b}, C(═O)R^{c}, C(═O)OR^{c}, C(O)NR^{b}R^{c}, C(═S)NR^{b}R^{c}, C₁-C₆-alkyl, C₃-C₆-cycloalkyl, C₂-C₆-alkenyl, C₂-C₆-alkynyl, C₁-C₆-alkoxy, C₂-C₆-alkenyloxy or C₂-C₆-alkynyloxy, which C-atoms of the aforementioned groups may be unsubstituted, partially or fully substituted with R^{aa}, wherein is as hereunder defined.

In a further embodiment, R^{2a} is, C(═O)OR^{c} or C(═O)NR^{b}R^{c}.

In another embodiment, R^{2a} is halogen, CN, C₁-C₆-alkyl, C₁-C₆-cycloalkyl, C₂-C₆-alkenyl, C₂-C₆-alkynyl, C₁-C₆-alkoxy, C₂-C₆-alkenyloxy or C₁-C₆-alkynyloxy, which C-atoms of the aforementioned groups may be unsubstituted, partially or fully substituted with R^{2aa}, wherein R^{2aa} is as hereunder defined.

In an embodiment, R^{2a} is Br, Cl or F, particularly Cl.

In another embodiment, R^{2a} is C₁-C₂-haloalkyl, preferably halomethyl such as CHF₂ or CF₃, particularly CF₃.

In an embodiment, R^{2aa} is halogen, C₁-C₆-alkyl, C₁-C₆-haloalkyl, C₁-C₆-alkoxy, C₁-C₆-haloalkoxy, C₂-C₄-alkenyl, C₂-C₄-alkynyl, C₃-C₆-cycloalkyl, CN, N(C₁-C₆-alkyl)(C₁-C₆-alkyl), C(═O)(O)ₚ(C₁-C₆-alkyl), C(═O)N(C₁-C₆-alkyl)(C₁-C₆-alkyl), S(O)ₘ(C₁-C₆-alkyl), SO₂N(C₁-C₆-alkyl)(C₁-C₆-alkyl), OSO₂(C₁-C₆-alkyl), N(C₁-C₆-alkyl)SO₂(C₁-C₆-alkyl), or S(═O)ₚ(═N(C₁-C₆-alkyl))(C₁-C₆-alkyl) or two geminally bound groups R^{2aa} together may form a group selected from ═O, ═S and ═N(C₁-C₆-alkyl).

In an embodiment, R^{2aa} is halogen, C₁-C₆-alkyl, C₁-C₆-haloalkyl, C₁-C₆-alkoxy, C₁-C₆-haloalkoxy, C₂-C₄-alkenyl, C₂-C₄-alkynyl, C₃-C₆-cycloalkyl, CN, N(C₁-C₆-alkyl)(C₁-C₆-alkyl). C(═O)(O)ₚ(C₁-C₆-alkyl), C(═O)N(C₁-C₆-alkyl)(C₁-C₆-alkyl), S(O)ₘ(C₁-C₆-alkyl), SO₂N(C₁-C₆-alkyl)(C₁-C₆-alkyl), OSO₂(C₁-C₆-alkyl), N(C₁-C₆-alkyl)SO₂(C₁-C₆-alkyl), or S(═O)ₚ(═N(C₁-C₆-alkyl))(C₁-C₆-alkyl). In another embodiment, two geminally bound groups R^{2aa} together may form a group selected from ═O, ═S and ═N(C₁-C₆-alkyl).

In an embodiment, compound of formula I is formula II-4, wherein

R¹ is alkyl, preferably CH₃;

W is O;

T is haloalkyl, preferably —CH₂—Cl;

R⁴ is D-25, preferably D-25a substituted with Cl;

In an embodiment, m is 0. In another embodiment, m is 1. In another embodiment, m is 2.

In an embodiment, n is 0. In another embodiment, n is 1. In another embodiment, n is 2.

In an embodiment, p is 0. In another embodiment, p is 1.

Preferred embodiment combinations are as shown in the following table:

Table of embodiment combinations

| No | W/T | R¹ | R² and R³ | R⁴ |
|---|---|---|---|---|
| EC-1 | <W-1> | <1-1> | <2-1> | <4-1> |
| EC-2 | <W-1> | <1-1> | <2-1> | <4-2> |
| EC-3 | <W-1> | <1-1> | <2-1> | <4-3> |
| EC-4 | <W-1> | <1-1> | <2-1> | <4-4> |
| EC-5 | <W-1> | <1-1> | <2-1> | <4-5> |
| EC-6 | <W-1> | <1-1> | <2-1> | <4-6> |
| EC-7 | <W-1> | <1-1> | <2-1> | <4-7> |
| EC-8 | <W-1> | <1-1> | <2-1> | <4-8> |
| EC-9 | <W-1> | <1-1> | <2-1> | <4-9 |
| EC-10 | <W-1> | <1-1> | <2-1> | <4-10> |
| EC-11 | <W-1> | <1-1> | <2-2> | <4-1> |
| EC-12 | <W-1> | <1-1> | <2-2> | <4-2> |
| EC-13 | <W-1> | <1-1> | <2-2> | <4-3> |
| EC-14 | <W-1> | <1-1> | <2-2> | <4-4> |
| EC-15 | <W-1> | <1-1> | <2-2> | <4-5> |
| EC-16 | <W-1> | <1-1> | <2-2> | <4-6> |
| EC-17 | <W-1> | <1-1> | <2-2> | <4-7> |
| EC-18 | <W-1> | <1-1> | <2-2> | <4-8> |
| EC-19 | <W-1> | <1-1> | <2-2> | <4-9 |
| EC-20 | <W-1> | <1-1> | <2-2> | <4-10> |
| EC-21 | <W-1> | <1-1> | <2-3> | <4-1> |
| EC-22 | <W-1> | <1-1> | <2-3> | <4-2> |
| EC-23 | <W-1> | <1-1> | <2-3> | <4-3> |
| EC-24 | <W-1> | <1-1> | <2-3> | <4-4> |
| EC-25 | <W-1> | <1-1> | <2-3> | <4-5> |
| EC-26 | <W-1> | <1-1> | <2-3> | <4-6> |
| EC-27 | <W-1> | <1-1> | <2-3> | <4-7> |
| EC-28 | <W-1> | <1-1> | <2-3> | <4-8> |
| EC-29 | <W-1> | <1-1> | <2-3> | <4-9 |
| EC-30 | <W-1> | <1-1> | <2-3> | <4-10> |
| EC-31 | <W-1> | <1-1> | <2-4> | <4-1> |
| EC-32 | <W-1> | <1-1> | <2-4> | <4-2> |
| EC-33 | <W-1> | <1-1> | <2-4> | <4-3> |
| EC-34 | <W-1> | <1-1> | <2-4> | <4-4> |
| EC-35 | <W-1> | <1-1> | <2-4> | <4-5> |
| EC-36 | <W-1> | <1-1> | <2-4> | <4-6> |
| EC-37 | <W-1> | <1-1> | <2-4> | <4-7> |
| EC-38 | <W-1> | <1-1> | <2-4> | <4-8> |
| EC-39 | <W-1> | <1-1> | <2-4> | <4-9 |
| EC-40 | <W-1> | <1-1> | <2-4> | <4-10> |
| EC-41 | <W-1> | <1-1> | <2-5> | <4-1> |
| EC-42 | <W-1> | <1-1> | <2-5> | <4-2> |
| EC-43 | <W-1> | <1-1> | <2-5> | <4-3> |
| EC-44 | <W-1> | <1-1> | <2-5> | <4-4> |
| EC-45 | <W-1> | <1-1> | <2-5> | <4-5> |
| EC-46 | <W-1> | <1-1> | <2-5> | <4-6> |
| EC-47 | <W-1> | <1-1> | <2-5> | <4-7> |
| EC-48 | <W-1> | <1-1> | <2-5> | <4-8> |
| EC-49 | <W-1> | <1-1> | <2-5> | <4-9 |
| EC-50 | <W-1> | <1-1> | <2-5> | <4-10> |
| EC-51 | <W-1> | <1-1> | <2-6> | <4-1> |
| EC-52 | <W-1> | <1-1> | <2-6> | <4-2> |
| EC-53 | <W-1> | <1-1> | <2-6> | <4-3> |
| EC-54 | <W-1> | <1-1> | <2-6> | <4-4> |
| EC-55 | <W-1> | <1-1> | <2-6> | <4-5> |
| EC-56 | <W-1> | <1-1> | <2-6> | <4-6> |
| EC-57 | <W-1> | <1-1> | <2-6> | <4-7> |
| EC-58 | <W-1> | <1-1> | <2-6> | <4-8> |
| EC-59 | <W-1> | <1-1> | <2-6> | <4-9 |
| EC-60 | <W-1> | <1-1> | <2-6> | <4-10> |
| EC-61 | <W-1> | <1-1> | <2-7> | <4-1> |
| EC-62 | <W-1> | <1-1> | <2-7> | <4-2> |
| EC-63 | <W-1> | <1-1> | <2-7> | <4-3> |
| EC-64 | <W-1> | <1-1> | <2-7> | <4-4> |
| EC-65 | <W-1> | <1-1> | <2-7> | <4-5> |
| EC-66 | <W-1> | <1-1> | <2-7> | <4-6> |
| EC-67 | <W-1> | <1-1> | <2-7> | <4-7> |
| EC-68 | <W-1> | <1-1> | <2-7> | <4-8> |
| EC-69 | <W-1> | <1-1> | <2-7> | <4-9 |
| EC-70 | <W-1> | <1-1> | <2-7> | <4-10> |
| EC-71 | <W-1> | <1-1> | <2-8> | <4-1> |
| EC-72 | <W-1> | <1-1> | <2-8> | <4-2> |
| EC-73 | <W-1> | <1-1> | <2-8> | <4-3> |
| EC-74 | <W-1> | <1-1> | <2-8> | <4-4> |
| EC-75 | <W-1> | <1-1> | <2-8> | <4-5> |
| EC-76 | <W-1> | <1-1> | <2-8> | <4-6> |
| EC-77 | <W-1> | <1-1> | <2-8> | <4-7> |
| EC-78 | <W-1> | <1-1> | <2-8> | <4-8> |
| EC-79 | <W-1> | <1-1> | <2-8> | <4-9 |
| EC-80 | <W-1> | <1-1> | <2-8> | <4-10> |
| EC-81 | <W-1> | <1-1> | <2-9> | <4-1> |
| EC-82 | <W-1> | <1-1> | <2-9> | <4-2> |
| EC-83 | <W-1> | <1-1> | <2-9> | <4-3> |
| EC-84 | <W-1> | <1-1> | <2-9> | <4-4> |
| EC-85 | <W-1> | <1-1> | <2-9> | <4-5> |
| EC-86 | <W-1> | <1-1> | <2-9> | <4-6> |
| EC-87 | <W-1> | <1-1> | <2-9> | <4-7> |
| EC-88 | <W-1> | <1-1> | <2-9> | <4-8> |
| EC-89 | <W-1> | <1-1> | <2-9> | <4-9 |
| EC-90 | <W-1> | <1-1> | <2-9> | <4-10> |
| EC-91 | <W-1> | <1-1> | <2-10> | <4-1> |
| EC-92 | <W-1> | <1-1> | <2-10> | <4-2> |
| EC-93 | <W-1> | <1-1> | <2-10> | <4-3> |
| EC-94 | <W-1> | <1-1> | <2-10> | <4-4> |
| EC-95 | <W-1> | <1-1> | <2-10> | <4-5> |
| EC-96 | <W-1> | <1-1> | <2-10> | <4-6> |
| EC-97 | <W-1> | <1-1> | <2-10> | <4-7> |
| EC-98 | <W-1> | <1-1> | <2-10> | <4-8> |
| EC-99 | <W-1> | <1-1> | <2-10> | <4-9 |
| EC-100 | <W-1> | <1-1> | <2-10> | <4-10> |
| EC-101 | <W-1> | <1-1> | <2-11> | <4-1> |
| EC-102 | <W-1> | <1-1> | <2-11> | <4-2> |
| EC-103 | <W-1> | <1-1> | <2-11> | <4-3> |
| EC-104 | <W-1> | <1-1> | <2-11> | <4-4> |
| EC-105 | <W-1> | <1-1> | <2-11> | <4-5> |
| EC-106 | <W-1> | <1-1> | <2-11> | <4-6> |
| EC-107 | <W-1> | <1-1> | <2-11> | <4-7> |
| EC-108 | <W-1> | <1-1> | <2-11> | <4-8> |
| EC-109 | <W-1> | <1-1> | <2-11> | <4-9 |
| EC-110 | <W-1> | <1-1> | <2-11> | <4-10> |
| EC-111 | <W-1> | <1-1> | <2-12> | <4-1> |
| EC-112 | <W-1> | <1-1> | <2-12> | <4-2> |
| EC-113 | <W-1> | <1-1> | <2-12> | <4-3> |
| EC-114 | <W-1> | <1-1> | <2-12> | <4-4> |
| EC-115 | <W-1> | <1-1> | <2-12> | <4-5> |
| EC-116 | <W-1> | <1-1> | <2-12> | <4-6> |
| EC-117 | <W-1> | <1-1> | <2-12> | <4-7> |
| EC-118 | <W-1> | <1-1> | <2-12> | <4-8> |
| EC-119 | <W-1> | <1-1> | <2-12> | <4-9 |
| EC-120 | <W-1> | <1-1> | <2-12> | <4-10> |
| EC-121 | <W-1> | <1-1> | <2-13> | <4-1> |
| EC-122 | <W-1> | <1-1> | <2-13> | <4-2> |
| EC-123 | <W-1> | <1-1> | <2-13> | <4-3> |
| EC-124 | <W-1> | <1-1> | <2-13> | <4-4> |
| EC-125 | <W-1> | <1-1> | <2-13> | <4-5> |
| EC-126 | <W-1> | <1-1> | <2-13> | <4-6> |
| EC-127 | <W-1> | <1-1> | <2-13> | <4-7> |
| EC-128 | <W-1> | <1-1> | <2-13> | <4-8> |
| EC-129 | <W-1> | <1-1> | <2-13> | <4-9 |
| EC-130 | <W-1> | <1-1> | <2-13> | <4-10> |
| EC-131 | <W-1> | <1-2> | <2-1> | <4-1> |
| EC-132 | <W-1> | <1-2> | <2-1> | <4-2> |
| EC-133 | <W-1> | <1-2> | <2-1> | <4-3> |
| EC-134 | <W-1> | <1-2> | <2-1> | <4-4> |
| EC-135 | <W-1> | <1-2> | <2-1> | <4-5> |
| EC-136 | <W-1> | <1-2> | <2-1> | <4-6> |
| EC-137 | <W-1> | <1-2> | <2-1> | <4-7> |
| EC-138 | <W-1> | <1-2> | <2-1> | <4-8> |
| EC-139 | <W-1> | <1-2> | <2-1> | <4-9 |
| EC-140 | <W-1> | <1-2> | <2-1> | <4-10> |
| EC-141 | <W-1> | <1-2> | <2-2> | <4-1> |
| EC-142 | <W-1> | <1-2> | <2-2> | <4-2> |
| EC-143 | <W-1> | <1-2> | <2-2> | <4-3> |
| EC-144 | <W-1> | <1-2> | <2-2> | <4-4> |
| EC-145 | <W-1> | <1-2> | <2-2> | <4-5> |
| EC-146 | <W-1> | <1-2> | <2-2> | <4-6> |
| EC-147 | <W-1> | <1-2> | <2-2> | <4-7> |
| EC-148 | <W-1> | <1-2> | <2-2> | <4-8> |
| EC-149 | <W-1> | <1-2> | <2-2> | <4-9 |
| EC-150 | <W-1> | <1-2> | <2-2> | <4-10> |
| EC-151 | <W-1> | <1-2> | <2-3> | <4-1> |
| EC-152 | <W-1> | <1-2> | <2-3> | <4-2> |
| EC-153 | <W-1> | <1-2> | <2-3> | <4-3> |
| EC-154 | <W-1> | <1-2> | <2-3> | <4-4> |
| EC-155 | <W-1> | <1-2> | <2-3> | <4-5> |
| EC-156 | <W-1> | <1-2> | <2-3> | <4-6> |
| EC-157 | <W-1> | <1-2> | <2-3> | <4-7> |
| EC-158 | <W-1> | <1-2> | <2-3> | <4-8> |
| EC-159 | <W-1> | <1-2> | <2-3> | <4-9 |
| EC-160 | <W-1> | <1-2> | <2-3> | <4-10> |
| EC-161 | <W-1> | <1-2> | <2-4> | <4-1> |
| EC-162 | <W-1> | <1-2> | <2-4> | <4-2> |
| EC-163 | <W-1> | <1-2> | <2-4> | <4-3> |
| EC-164 | <W-1> | <1-2> | <2-4> | <4-4> |
| EC-165 | <W-1> | <1-2> | <2-4> | <4-5> |
| EC-166 | <W-1> | <1-2> | <2-4> | <4-6> |
| EC-167 | <W-1> | <1-2> | <2-4> | <4-7> |
| EC-168 | <W-1> | <1-2> | <2-4> | <4-8> |
| EC-169 | <W-1> | <1-2> | <2-4> | <4-9 |
| EC-170 | <W-1> | <1-2> | <2-4> | <4-10> |
| EC-171 | <W-1> | <1-2> | <2-5> | <4-1> |
| EC-172 | <W-1> | <1-2> | <2-5> | <4-2> |
| EC-173 | <W-1> | <1-2> | <2-5> | <4-3> |
| EC-174 | <W-1> | <1-2> | <2-5> | <4-4> |
| EC-175 | <W-1> | <1-2> | <2-5> | <4-5> |
| EC-176 | <W-1> | <1-2> | <2-5> | <4-6> |
| EC-177 | <W-1> | <1-2> | <2-5> | <4-7> |
| EC-178 | <W-1> | <1-2> | <2-5> | <4-8> |
| EC-179 | <W-1> | <1-2> | <2-5> | <4-9 |
| EC-180 | <W-1> | <1-2> | <2-5> | <4-10> |
| EC-181 | <W-1> | <1-2> | <2-6> | <4-1> |
| EC-182 | <W-1> | <1-2> | <2-6> | <4-2> |
| EC-183 | <W-1> | <1-2> | <2-6> | <4-3> |
| EC-184 | <W-1> | <1-2> | <2-6> | <4-4> |
| EC-185 | <W-1> | <1-2> | <2-6> | <4-5> |
| EC-186 | <W-1> | <1-2> | <2-6> | <4-6> |
| EC-187 | <W-1> | <1-2> | <2-6> | <4-7> |
| EC-188 | <W-1> | <1-2> | <2-6> | <4-8> |
| EC-189 | <W-1> | <1-2> | <2-6> | <4-9 |
| EC-190 | <W-1> | <1-2> | <2-6> | <4-10> |
| EC-191 | <W-1> | <1-2> | <2-7> | <4-1> |
| EC-192 | <W-1> | <1-2> | <2-7> | <4-2> |
| EC-193 | <W-1> | <1-2> | <2-7> | <4-3> |
| EC-194 | <W-1> | <1-2> | <2-7> | <4-4> |
| EC-195 | <W-1> | <1-2> | <2-7> | <4-5> |
| EC-196 | <W-1> | <1-2> | <2-7> | <4-6> |
| EC-197 | <W-1> | <1-2> | <2-7> | <4-7> |
| EC-198 | <W-1> | <1-2> | <2-7> | <4-8> |
| EC-199 | <W-1> | <1-2> | <2-7> | <4-9 |
| EC-200 | <W-1> | <1-2> | <2-7> | <4-10> |
| EC-201 | <W-1> | <1-2> | <2-8> | <4-1> |
| EC-202 | <W-1> | <1-2> | <2-8> | <4-2> |
| EC-203 | <W-1> | <1-2> | <2-8> | <4-3> |
| EC-204 | <W-1> | <1-2> | <2-8> | <4-4> |
| EC-205 | <W-1> | <1-2> | <2-8> | <4-5> |
| EC-206 | <W-1> | <1-2> | <2-8> | <4-6> |
| EC-207 | <W-1> | <1-2> | <2-8> | <4-7> |
| EC-208 | <W-1> | <1-2> | <2-8> | <4-8> |
| EC-209 | <W-1> | <1-2> | <2-8> | <4-9 |
| EC-210 | <W-1> | <1-2> | <2-8> | <4-10> |
| EC-211 | <W-1> | <1-2> | <2-9> | <4-1> |
| EC-212 | <W-1> | <1-2> | <2-9> | <4-2> |
| EC-213 | <W-1> | <1-2> | <2-9> | <4-3> |
| EC-214 | <W-1> | <1-2> | <2-9> | <4-4> |
| EC-215 | <W-1> | <1-2> | <2-9> | <4-5> |
| EC-216 | <W-1> | <1-2> | <2-9> | <4-6> |
| EC-217 | <W-1> | <1-2> | <2-9> | <4-7> |
| EC-218 | <W-1> | <1-2> | <2-9> | <4-8> |
| EC-219 | <W-1> | <1-2> | <2-9> | <4-9 |
| EC-220 | <W-1> | <1-2> | <2-9> | <4-10> |
| EC-221 | <W-1> | <1-2> | <2-10> | <4-1> |
| EC-222 | <W-1> | <1-2> | <2-10> | <4-2> |
| EC-223 | <W-1> | <1-2> | <2-10> | <4-3> |
| EC-224 | <W-1> | <1-2> | <2-10> | <4-4> |
| EC-225 | <W-1> | <1-2> | <2-10> | <4-5> |
| EC-226 | <W-1> | <1-2> | <2-10> | <4-6> |
| EC-227 | <W-1> | <1-2> | <2-10> | <4-7> |
| EC-228 | <W-1> | <1-2> | <2-10> | <4-8> |
| EC-229 | <W-1> | <1-2> | <2-10> | <4-9 |
| EC-230 | <W-1> | <1-2> | <2-10> | <4-10> |
| EC-231 | <W-1> | <1-2> | <2-11> | <4-1> |
| EC-232 | <W-1> | <1-2> | <2-11> | <4-2> |
| EC-233 | <W-1> | <1-2> | <2-11> | <4-3> |
| EC-234 | <W-1> | <1-2> | <2-11> | <4-4> |
| EC-235 | <W-1> | <1-2> | <2-11> | <4-5> |
| EC-236 | <W-1> | <1-2> | <2-11> | <4-6> |
| EC-237 | <W-1> | <1-2> | <2-11> | <4-7> |
| EC-238 | <W-1> | <1-2> | <2-11> | <4-8> |
| EC-239 | <W-1> | <1-2> | <2-11> | <4-9 |
| EC-240 | <W-1> | <1-2> | <2-11> | <4-10> |
| EC-241 | <W-1> | <1-2> | <2-12> | <4-1> |
| EC-242 | <W-1> | <1-2> | <2-12> | <4-2> |
| EC-243 | <W-1> | <1-2> | <2-12> | <4-3> |
| EC-244 | <W-1> | <1-2> | <2-12> | <4-4> |
| EC-245 | <W-1> | <1-2> | <2-12> | <4-5> |
| EC-246 | <W-1> | <1-2> | <2-12> | <4-6> |
| EC-247 | <W-1> | <1-2> | <2-12> | <4-7> |
| EC-248 | <W-1> | <1-2> | <2-12> | <4-8> |
| EC-249 | <W-1> | <1-2> | <2-12> | <4-9 |
| EC-250 | <W-1> | <1-2> | <2-12> | <4-10> |
| EC-251 | <W-1> | <1-2> | <2-13> | <4-1> |
| EC-252 | <W-1> | <1-2> | <2-13> | <4-2> |
| EC-253 | <W-1> | <1-2> | <2-13> | <4-3> |
| EC-254 | <W-1> | <1-2> | <2-13> | <4-4> |
| EC-255 | <W-1> | <1-2> | <2-13> | <4-5> |
| EC-256 | <W-1> | <1-2> | <2-13> | <4-6> |
| EC-257 | <W-1> | <1-2> | <2-13> | <4-7> |
| EC-258 | <W-1> | <1-2> | <2-13> | <4-8> |
| EC-259 | <W-1> | <1-2> | <2-13> | <4-9 |
| EC-260 | <W-1> | <1-2> | <2-13> | <4-10> |
| EC-261 | <W-1> | <1-3> | <2-1> | <4-1> |
| EC-262 | <W-1> | <1-3> | <2-1> | <4-2> |
| EC-263 | <W-1> | <1-3> | <2-1> | <4-3> |
| EC-264 | <W-1> | <1-3> | <2-1> | <4-4> |
| EC-265 | <W-1> | <1-3> | <2-1> | <4-5> |
| EC-266 | <W-1> | <1-3> | <2-1> | <4-6> |
| EC-267 | <W-1> | <1-3> | <2-1> | <4-7> |
| EC-268 | <W-1> | <1-3> | <2-1> | <4-8> |
| EC-269 | <W-1> | <1-3> | <2-1> | <4-9 |
| EC-270 | <W-1> | <1-3> | <2-1> | <4-10> |
| EC-271 | <W-1> | <1-3> | <2-2> | <4-1> |
| EC-272 | <W-1> | <1-3> | <2-2> | <4-2> |
| EC-273 | <W-1> | <1-3> | <2-2> | <4-3> |
| EC-274 | <W-1> | <1-3> | <2-2> | <4-4> |
| EC-275 | <W-1> | <1-3> | <2-2> | <4-5> |
| EC-276 | <W-1> | <1-3> | <2-2> | <4-6> |
| EC-277 | <W-1> | <1-3> | <2-2> | <4-7> |
| EC-278 | <W-1> | <1-3> | <2-2> | <4-8> |
| EC-279 | <W-1> | <1-3> | <2-2> | <4-9 |
| EC-280 | <W-1> | <1-3> | <2-2> | <4-10> |
| EC-281 | <W-1> | <1-3> | <2-3> | <4-1> |
| EC-282 | <W-1> | <1-3> | <2-3> | <4-2> |
| EC-283 | <W-1> | <1-3> | <2-3> | <4-3> |
| EC-284 | <W-1> | <1-3> | <2-3> | <4-4> |
| EC-285 | <W-1> | <1-3> | <2-3> | <4-5> |
| EC-286 | <W-1> | <1-3> | <2-3> | <4-6> |
| EC-287 | <W-1> | <1-3> | <2-3> | <4-7> |
| EC-288 | <W-1> | <1-3> | <2-3> | <4-8> |
| EC-289 | <W-1> | <1-3> | <2-3> | <4-9 |
| EC-290 | <W-1> | <1-3> | <2-3> | <4-10> |
| EC-291 | <W-1> | <1-3> | <2-4> | <4-1> |
| EC-292 | <W-1> | <1-3> | <2-4> | <4-2> |
| EC-293 | <W-1> | <1-3> | <2-4> | <4-3> |
| EC-294 | <W-1> | <1-3> | <2-4> | <4-4> |
| EC-295 | <W-1> | <1-3> | <2-4> | <4-5> |
| EC-296 | <W-1> | <1-3> | <2-4> | <4-6> |
| EC-297 | <W-1> | <1-3> | <2-4> | <4-7> |
| EC-298 | <W-1> | <1-3> | <2-4> | <4-8> |
| EC-299 | <W-1> | <1-3> | <2-4> | <4-9 |
| EC-300 | <W-1> | <1-3> | <2-4> | <4-10> |
| EC-301 | <W-1> | <1-3> | <2-5> | <4-1> |
| EC-302 | <W-1> | <1-3> | <2-5> | <4-2> |
| EC-303 | <W-1> | <1-3> | <2-5> | <4-3> |
| EC-304 | <W-1> | <1-3> | <2-5> | <4-4> |
| EC-305 | <W-1> | <1-3> | <2-5> | <4-5> |
| EC-306 | <W-1> | <1-3> | <2-5> | <4-6> |
| EC-307 | <W-1> | <1-3> | <2-5> | <4-7> |
| EC-308 | <W-1> | <1-3> | <2-5> | <4-8> |
| EC-309 | <W-1> | <1-3> | <2-5> | <4-9 |
| EC-310 | <W-1> | <1-3> | <2-5> | <4-10> |
| EC-311 | <W-1> | <1-3> | <2-6> | <4-1> |
| EC-312 | <W-1> | <1-3> | <2-6> | <4-2> |
| EC-313 | <W-1> | <1-3> | <2-6> | <4-3> |
| EC-314 | <W-1> | <1-3> | <2-6> | <4-4> |
| EC-315 | <W-1> | <1-3> | <2-6> | <4-5> |
| EC-316 | <W-1> | <1-3> | <2-6> | <4-6> |
| EC-317 | <W-1> | <1-3> | <2-6> | <4-7> |
| EC-318 | <W-1> | <1-3> | <2-6> | <4-8> |
| EC-319 | <W-1> | <1-3> | <2-6> | <4-9 |
| EC-320 | <W-1> | <1-3> | <2-6> | <4-10> |
| EC-321 | <W-1> | <1-3> | <2-7> | <4-1> |
| EC-322 | <W-1> | <1-3> | <2-7> | <4-2> |
| EC-323 | <W-1> | <1-3> | <2-7> | <4-3> |
| EC-324 | <W-1> | <1-3> | <2-7> | <4-4> |
| EC-325 | <W-1> | <1-3> | <2-7> | <4-5> |
| EC-326 | <W-1> | <1-3> | <2-7> | <4-6> |
| EC-327 | <W-1> | <1-3> | <2-7> | <4-7> |
| EC-328 | <W-1> | <1-3> | <2-7> | <4-8> |
| EC-329 | <W-1> | <1-3> | <2-7> | <4-9 |
| EC-330 | <W-1> | <1-3> | <2-7> | <4-10> |
| EC-331 | <W-1> | <1-3> | <2-8> | <4-1> |
| EC-332 | <W-1> | <1-3> | <2-8> | <4-2> |
| EC-333 | <W-1> | <1-3> | <2-8> | <4-3> |
| EC-334 | <W-1> | <1-3> | <2-8> | <4-4> |
| EC-335 | <W-1> | <1-3> | <2-8> | <4-5> |
| EC-336 | <W-1> | <1-3> | <2-8> | <4-6> |
| EC-337 | <W-1> | <1-3> | <2-8> | <4-7> |
| EC-338 | <W-1> | <1-3> | <2-8> | <4-8> |
| EC-339 | <W-1> | <1-3> | <2-8> | <4-9 |
| EC-340 | <W-1> | <1-3> | <2-8> | <4-10> |
| EC-341 | <W-1> | <1-3> | <2-9> | <4-1> |
| EC-342 | <W-1> | <1-3> | <2-9> | <4-2> |
| EC-343 | <W-1> | <1-3> | <2-9> | <4-3> |
| EC-344 | <W-1> | <1-3> | <2-9> | <4-4> |
| EC-345 | <W-1> | <1-3> | <2-9> | <4-5> |
| EC-346 | <W-1> | <1-3> | <2-9> | <4-6> |
| EC-347 | <W-1> | <1-3> | <2-9> | <4-7> |
| EC-348 | <W-1> | <1-3> | <2-9> | <4-8> |
| EC-349 | <W-1> | <1-3> | <2-9> | <4-9 |
| EC-350 | <W-1> | <1-3> | <2-9> | <4-10> |
| EC-351 | <W-1> | <1-3> | <2-10> | <4-1> |
| EC-352 | <W-1> | <1-3> | <2-10> | <4-2> |
| EC-353 | <W-1> | <1-3> | <2-10> | <4-3> |
| EC-354 | <W-1> | <1-3> | <2-10> | <4-4> |
| EC-355 | <W-1> | <1-3> | <2-10> | <4-5> |
| EC-356 | <W-1> | <1-3> | <2-10> | <4-6> |
| EC-357 | <W-1> | <1-3> | <2-10> | <4-7> |
| EC-358 | <W-1> | <1-3> | <2-10> | <4-8> |
| EC-359 | <W-1> | <1-3> | <2-10> | <4-9 |
| EC-360 | <W-1> | <1-3> | <2-10> | <4-10> |
| EC-361 | <W-1> | <1-3> | <2-11> | <4-1> |
| EC-362 | <W-1> | <1-3> | <2-11> | <4-2> |
| EC-363 | <W-1> | <1-3> | <2-11> | <4-3> |
| EC-364 | <W-1> | <1-3> | <2-11> | <4-4> |
| EC-365 | <W-1> | <1-3> | <2-11> | <4-5> |
| EC-366 | <W-1> | <1-3> | <2-11> | <4-6> |
| EC-367 | <W-1> | <1-3> | <2-11> | <4-7> |
| EC-368 | <W-1> | <1-3> | <2-11> | <4-8> |
| EC-369 | <W-1> | <1-3> | <2-11> | <4-9 |
| EC-370 | <W-1> | <1-3> | <2-11> | <4-10> |
| EC-371 | <W-1> | <1-3> | <2-12> | <4-1> |
| EC-372 | <W-1> | <1-3> | <2-12> | <4-2> |
| EC-373 | <W-1> | <1-3> | <2-12> | <4-3> |
| EC-374 | <W-1> | <1-3> | <2-12> | <4-4> |
| EC-375 | <W-1> | <1-3> | <2-12> | <4-5> |
| EC-376 | <W-1> | <1-3> | <2-12> | <4-6> |
| EC-377 | <W-1> | <1-3> | <2-12> | <4-7> |
| EC-378 | <W-1> | <1-3> | <2-12> | <4-8> |
| EC-379 | <W-1> | <1-3> | <2-12> | <4-9 |
| EC-380 | <W-1> | <1-3> | <2-12> | <4-10> |
| EC-381 | <W-1> | <1-3> | <2-13> | <4-1> |
| EC-382 | <W-1> | <1-3> | <2-13> | <4-2> |
| EC-383 | <W-1> | <1-3> | <2-13> | <4-3> |
| EC-384 | <W-1> | <1-3> | <2-13> | <4-4> |
| EC-385 | <W-1> | <1-3> | <2-13> | <4-5> |
| EC-386 | <W-1> | <1-3> | <2-13> | <4-6> |
| EC-387 | <W-1> | <1-3> | <2-13> | <4-7> |
| EC-388 | <W-1> | <1-3> | <2-13> | <4-8> |
| EC-389 | <W-1> | <1-3> | <2-13> | <4-9 |
| EC-390 | <W-1> | <1-3> | <2-13> | <4-10> |
| EC-391 | <W-1> | <1-4> | <2-1> | <4-1> |
| EC-392 | <W-1> | <1-4> | <2-1> | <4-2> |
| EC-393 | <W-1> | <1-4> | <2-1> | <4-3> |
| EC-394 | <W-1> | <1-4> | <2-1> | <4-4> |
| EC-395 | <W-1> | <1-4> | <2-1> | <4-5> |
| EC-396 | <W-1> | <1-4> | <2-1> | <4-6> |
| EC-397 | <W-1> | <1-4> | <2-1> | <4-7> |
| EC-398 | <W-1> | <1-4> | <2-1> | <4-8> |
| EC-399 | <W-1> | <1-4> | <2-1> | <4-9 |
| EC-400 | <W-1> | <1-4> | <2-1> | <4-10> |
| EC-401 | <W-1> | <1-4> | <2-2> | <4-1> |
| EC-402 | <W-1> | <1-4> | <2-2> | <4-2> |
| EC-403 | <W-1> | <1-4> | <2-2> | <4-3> |
| EC-404 | <W-1> | <1-4> | <2-2> | <4-4> |
| EC-405 | <W-1> | <1-4> | <2-2> | <4-5> |
| EC-406 | <W-1> | <1-4> | <2-2> | <4-6> |
| EC-407 | <W-1> | <1-4> | <2-2> | <4-7> |
| EC-408 | <W-1> | <1-4> | <2-2> | <4-8> |
| EC-409 | <W-1> | <1-4> | <2-2> | <4-9 |
| EC-410 | <W-1> | <1-4> | <2-2> | <4-10> |
| EC-411 | <W-1> | <1-4> | <2-3> | <4-1> |
| EC-412 | <W-1> | <1-4> | <2-3> | <4-2> |
| EC-413 | <W-1> | <1-4> | <2-3> | <4-3> |
| EC-414 | <W-1> | <1-4> | <2-3> | <4-4> |
| EC-415 | <W-1> | <1-4> | <2-3> | <4-5> |
| EC-416 | <W-1> | <1-4> | <2-3> | <4-6> |
| EC-417 | <W-1> | <1-4> | <2-3> | <4-7> |
| EC-418 | <W-1> | <1-4> | <2-3> | <4-8> |
| EC-419 | <W-1> | <1-4> | <2-3> | <4-9 |
| EC-420 | <W-1> | <1-4> | <2-3> | <4-10> |
| EC-421 | <W-1> | <1-4> | <2-4> | <4-1> |
| EC-422 | <W-1> | <1-4> | <2-4> | <4-2> |
| EC-423 | <W-1> | <1-4> | <2-4> | <4-3> |
| EC-424 | <W-1> | <1-4> | <2-4> | <4-4> |
| EC-425 | <W-1> | <1-4> | <2-4> | <4-5> |
| EC-426 | <W-1> | <1-4> | <2-4> | <4-6> |
| EC-427 | <W-1> | <1-4> | <2-4> | <4-7> |
| EC-428 | <W-1> | <1-4> | <2-4> | <4-8> |
| EC-429 | <W-1> | <1-4> | <2-4> | <4-9 |
| EC-430 | <W-1> | <1-4> | <2-4> | <4-10> |
| EC-431 | <W-1> | <1-4> | <2-5> | <4-1> |
| EC-432 | <W-1> | <1-4> | <2-5> | <4-2> |
| EC-433 | <W-1> | <1-4> | <2-5> | <4-3> |
| EC-434 | <W-1> | <1-4> | <2-5> | <4-4> |
| EC-435 | <W-1> | <1-4> | <2-5> | <4-5> |
| EC-436 | <W-1> | <1-4> | <2-5> | <4-6> |
| EC-437 | <W-1> | <1-4> | <2-5> | <4-7> |
| EC-438 | <W-1> | <1-4> | <2-5> | <4-8> |
| EC-439 | <W-1> | <1-4> | <2-5> | <4-9 |
| EC-440 | <W-1> | <1-4> | <2-5> | <4-10> |
| EC-441 | <W-1> | <1-4> | <2-6> | <4-1> |
| EC-442 | <W-1> | <1-4> | <2-6> | <4-2> |
| EC-443 | <W-1> | <1-4> | <2-6> | <4-3> |
| EC-444 | <W-1> | <1-4> | <2-6> | <4-4> |
| EC-445 | <W-1> | <1-4> | <2-6> | <4-5> |
| EC-446 | <W-1> | <1-4> | <2-6> | <4-6> |
| EC-447 | <W-1> | <1-4> | <2-6> | <4-7> |
| EC-448 | <W-1> | <1-4> | <2-6> | <4-8> |
| EC-449 | <W-1> | <1-4> | <2-6> | <4-9 |
| EC-450 | <W-1> | <1-4> | <2-6> | <4-10> |
| EC-451 | <W-1> | <1-4> | <2-7> | <4-1> |
| EC-452 | <W-1> | <1-4> | <2-7> | <4-2> |
| EC-453 | <W-1> | <1-4> | <2-7> | <4-3> |
| EC-454 | <W-1> | <1-4> | <2-7> | <4-4> |
| EC-455 | <W-1> | <1-4> | <2-7> | <4-5> |
| EC-456 | <W-1> | <1-4> | <2-7> | <4-6> |
| EC-457 | <W-1> | <1-4> | <2-7> | <4-7> |
| EC-458 | <W-1> | <1-4> | <2-7> | <4-8> |
| EC-459 | <W-1> | <1-4> | <2-7> | <4-9 |
| EC-460 | <W-1> | <1-4> | <2-7> | <4-10> |
| EC-461 | <W-1> | <1-4> | <2-8> | <4-1> |
| EC-462 | <W-1> | <1-4> | <2-8> | <4-2> |
| EC-463 | <W-1> | <1-4> | <2-8> | <4-3> |
| EC-464 | <W-1> | <1-4> | <2-8> | <4-4> |
| EC-465 | <W-1> | <1-4> | <2-8> | <4-5> |
| EC-466 | <W-1> | <1-4> | <2-8> | <4-6> |
| EC-467 | <W-1> | <1-4> | <2-8> | <4-7> |
| EC-468 | <W-1> | <1-4> | <2-8> | <4-8> |
| EC-469 | <W-1> | <1-4> | <2-8> | <4-9 |
| EC-470 | <W-1> | <1-4> | <2-8> | <4-10> |
| EC-471 | <W-1> | <1-4> | <2-9> | <4-1> |
| EC-472 | <W-1> | <1-4> | <2-9> | <4-2> |
| EC-473 | <W-1> | <1-4> | <2-9> | <4-3> |
| EC-474 | <W-1> | <1-4> | <2-9> | <4-4> |
| EC-475 | <W-1> | <1-4> | <2-9> | <4-5> |
| EC-476 | <W-1> | <1-4> | <2-9> | <4-6> |
| EC-477 | <W-1> | <1-4> | <2-9> | <4-7> |
| EC-478 | <W-1> | <1-4> | <2-9> | <4-8> |
| EC-479 | <W-1> | <1-4> | <2-9> | <4-9 |
| EC-480 | <W-1> | <1-4> | <2-9> | <4-10> |
| EC-481 | <W-1> | <1-4> | <2-10> | <4-1> |
| EC-482 | <W-1> | <1-4> | <2-10> | <4-2> |
| EC-483 | <W-1> | <1-4> | <2-10> | <4-3> |
| EC-484 | <W-1> | <1-4> | <2-10> | <4-4> |
| EC-485 | <W-1> | <1-4> | <2-10> | <4-5> |
| EC-486 | <W-1> | <1-4> | <2-10> | <4-6> |
| EC-487 | <W-1> | <1-4> | <2-10> | <4-7> |
| EC-488 | <W-1> | <1-4> | <2-10> | <4-8> |
| EC-489 | <W-1> | <1-4> | <2-10> | <4-9 |
| EC-490 | <W-1> | <1-4> | <2-10> | <4-10> |
| EC-491 | <W-1> | <1-4> | <2-11> | <4-1> |
| EC-492 | <W-1> | <1-4> | <2-11> | <4-2> |
| EC-493 | <W-1> | <1-4> | <2-11> | <4-3> |
| EC-494 | <W-1> | <1-4> | <2-11> | <4-4> |
| EC-495 | <W-1> | <1-4> | <2-11> | <4-5> |
| EC-496 | <W-1> | <1-4> | <2-11> | <4-6> |
| EC-497 | <W-1> | <1-4> | <2-11> | <4-7> |
| EC-498 | <W-1> | <1-4> | <2-11> | <4-8> |
| EC-499 | <W-1> | <1-4> | <2-11> | <4-9 |
| EC-500 | <W-1> | <1-4> | <2-11> | <4-10> |
| EC-501 | <W-1> | <1-4> | <2-12> | <4-1> |
| EC-502 | <W-1> | <1-4> | <2-12> | <4-2> |
| EC-503 | <W-1> | <1-4> | <2-12> | <4-3> |
| EC-504 | <W-1> | <1-4> | <2-12> | <4-4> |
| EC-505 | <W-1> | <1-4> | <2-12> | <4-5> |
| EC-506 | <W-1> | <1-4> | <2-12> | <4-6> |
| EC-507 | <W-1> | <1-4> | <2-12> | <4-7> |
| EC-508 | <W-1> | <1-4> | <2-12> | <4-8> |
| EC-509 | <W-1> | <1-4> | <2-12> | <4-9 |
| EC-510 | <W-1> | <1-4> | <2-12> | <4-10> |
| EC-511 | <W-1> | <1-4> | <2-13> | <4-1> |
| EC-512 | <W-1> | <1-4> | <2-13> | <4-2> |
| EC-513 | <W-1> | <1-4> | <2-13> | <4-3> |
| EC-514 | <W-1> | <1-4> | <2-13> | <4-4> |
| EC-515 | <W-1> | <1-4> | <2-13> | <4-5> |
| EC-516 | <W-1> | <1-4> | <2-13> | <4-6> |
| EC-517 | <W-1> | <1-4> | <2-13> | <4-7> |
| EC-518 | <W-1> | <1-4> | <2-13> | <4-8> |
| EC-519 | <W-1> | <1-4> | <2-13> | <4-9 |
| EC-520 | <W-1> | <1-4> | <2-13> | <4-10> |
| EC-521 | <W-1> | <1-5> | <2-1> | <4-1> |
| EC-522 | <W-1> | <1-5> | <2-1> | <4-2> |
| EC-523 | <W-1> | <1-5> | <2-1> | <4-3> |
| EC-524 | <W-1> | <1-5> | <2-1> | <4-4> |
| EC-525 | <W-1> | <1-5> | <2-1> | <4-5> |
| EC-526 | <W-1> | <1-5> | <2-1> | <4-6> |
| EC-527 | <W-1> | <1-5> | <2-1> | <4-7> |
| EC-528 | <W-1> | <1-5> | <2-1> | <4-8> |
| EC-529 | <W-1> | <1-5> | <2-1> | <4-9 |
| EC-530 | <W-1> | <1-5> | <2-1> | <4-10> |
| EC-531 | <W-1> | <1-5> | <2-2> | <4-1> |
| EC-532 | <W-1> | <1-5> | <2-2> | <4-2> |
| EC-533 | <W-1> | <1-5> | <2-2> | <4-3> |
| EC-534 | <W-1> | <1-5> | <2-2> | <4-4> |
| EC-535 | <W-1> | <1-5> | <2-2> | <4-5> |
| EC-536 | <W-1> | <1-5> | <2-2> | <4-6> |
| EC-537 | <W-1> | <1-5> | <2-2> | <4-7> |
| EC-538 | <W-1> | <1-5> | <2-2> | <4-8> |
| EC-539 | <W-1> | <1-5> | <2-2> | <4-9 |
| EC-540 | <W-1> | <1-5> | <2-2> | <4-10> |
| EC-541 | <W-1> | <1-5> | <2-3> | <4-1> |
| EC-542 | <W-1> | <1-5> | <2-3> | <4-2> |
| EC-543 | <W-1> | <1-5> | <2-3> | <4-3> |
| EC-544 | <W-1> | <1-5> | <2-3> | <4-4> |
| EC-545 | <W-1> | <1-5> | <2-3> | <4-5> |
| EC-546 | <W-1> | <1-5> | <2-3> | <4-6> |
| EC-547 | <W-1> | <1-5> | <2-3> | <4-7> |
| EC-548 | <W-1> | <1-5> | <2-3> | <4-8> |
| EC-549 | <W-1> | <1-5> | <2-3> | <4-9 |
| EC-550 | <W-1> | <1-5> | <2-3> | <4-10> |
| EC-551 | <W-1> | <1-5> | <2-4> | <4-1> |
| EC-552 | <W-1> | <1-5> | <2-4> | <4-2> |
| EC-553 | <W-1> | <1-5> | <2-4> | <4-3> |
| EC-554 | <W-1> | <1-5> | <2-4> | <4-4> |
| EC-555 | <W-1> | <1-5> | <2-4> | <4-5> |
| EC-556 | <W-1> | <1-5> | <2-4> | <4-6> |
| EC-557 | <W-1> | <1-5> | <2-4> | <4-7> |
| EC-558 | <W-1> | <1-5> | <2-4> | <4-8> |
| EC-559 | <W-1> | <1-5> | <2-4> | <4-9 |
| EC-560 | <W-1> | <1-5> | <2-4> | <4-10> |
| EC-561 | <W-1> | <1-5> | <2-5> | <4-1> |
| EC-562 | <W-1> | <1-5> | <2-5> | <4-2> |
| EC-563 | <W-1> | <1-5> | <2-5> | <4-3> |
| EC-564 | <W-1> | <1-5> | <2-5> | <4-4> |
| EC-565 | <W-1> | <1-5> | <2-5> | <4-5> |
| EC-566 | <W-1> | <1-5> | <2-5> | <4-6> |
| EC-567 | <W-1> | <1-5> | <2-5> | <4-7> |
| EC-568 | <W-1> | <1-5> | <2-5> | <4-8> |
| EC-569 | <W-1> | <1-5> | <2-5> | <4-9 |
| EC-570 | <W-1> | <1-5> | <2-5> | <4-10> |
| EC-571 | <W-1> | <1-5> | <2-6> | <4-1> |
| EC-572 | <W-1> | <1-5> | <2-6> | <4-2> |
| EC-573 | <W-1> | <1-5> | <2-6> | <4-3> |
| EC-574 | <W-1> | <1-5> | <2-6> | <4-4> |
| EC-575 | <W-1> | <1-5> | <2-6> | <4-5> |
| EC-576 | <W-1> | <1-5> | <2-6> | <4-6> |
| EC-577 | <W-1> | <1-5> | <2-6> | <4-7> |
| EC-578 | <W-1> | <1-5> | <2-6> | <4-8> |
| EC-579 | <W-1> | <1-5> | <2-6> | <4-9 |
| EC-580 | <W-1> | <1-5> | <2-6> | <4-10> |
| EC-581 | <W-1> | <1-5> | <2-7> | <4-1> |
| EC-582 | <W-1> | <1-5> | <2-7> | <4-2> |
| EC-583 | <W-1> | <1-5> | <2-7> | <4-3> |
| EC-584 | <W-1> | <1-5> | <2-7> | <4-4> |
| EC-585 | <W-1> | <1-5> | <2-7> | <4-5> |
| EC-586 | <W-1> | <1-5> | <2-7> | <4-6> |
| EC-587 | <W-1> | <1-5> | <2-7> | <4-7> |
| EC-588 | <W-1> | <1-5> | <2-7> | <4-8> |
| EC-589 | <W-1> | <1-5> | <2-7> | <4-9 |
| EC-590 | <W-1> | <1-5> | <2-7> | <4-10> |
| EC-591 | <W-1> | <1-5> | <2-8> | <4-1> |
| EC-592 | <W-1> | <1-5> | <2-8> | <4-2> |
| EC-593 | <W-1> | <1-5> | <2-8> | <4-3> |
| EC-594 | <W-1> | <1-5> | <2-8> | <4-4> |
| EC-595 | <W-1> | <1-5> | <2-8> | <4-5> |
| EC-596 | <W-1> | <1-5> | <2-8> | <4-6> |
| EC-597 | <W-1> | <1-5> | <2-8> | <4-7> |
| EC-598 | <W-1> | <1-5> | <2-8> | <4-8> |
| EC-599 | <W-1> | <1-5> | <2-8> | <4-9 |
| EC-600 | <W-1> | <1-5> | <2-8> | <4-10> |
| EC-601 | <W-1> | <1-5> | <2-9> | <4-1> |
| EC-602 | <W-1> | <1-5> | <2-9> | <4-2> |
| EC-603 | <W-1> | <1-5> | <2-9> | <4-3> |
| EC-604 | <W-1> | <1-5> | <2-9> | <4-4> |
| EC-605 | <W-1> | <1-5> | <2-9> | <4-5> |
| EC-606 | <W-1> | <1-5> | <2-9> | <4-6> |
| EC-607 | <W-1> | <1-5> | <2-9> | <4-7> |
| EC-608 | <W-1> | <1-5> | <2-9> | <4-8> |
| EC-609 | <W-1> | <1-5> | <2-9> | <4-9 |
| EC-610 | <W-1> | <1-5> | <2-9> | <4-10> |
| EC-611 | <W-1> | <1-5> | <2-10> | <4-1> |
| EC-612 | <W-1> | <1-5> | <2-10> | <4-2> |
| EC-613 | <W-1> | <1-5> | <2-10> | <4-3> |
| EC-614 | <W-1> | <1-5> | <2-10> | <4-4> |
| EC-615 | <W-1> | <1-5> | <2-10> | <4-5> |
| EC-616 | <W-1> | <1-5> | <2-10> | <4-6> |
| EC-617 | <W-1> | <1-5> | <2-10> | <4-7> |
| EC-618 | <W-1> | <1-5> | <2-10> | <4-8> |
| EC-619 | <W-1> | <1-5> | <2-10> | <4-9 |
| EC-620 | <W-1> | <1-5> | <2-10> | <4-10> |
| EC-621 | <W-1> | <1-5> | <2-11> | <4-1> |
| EC-622 | <W-1> | <1-5> | <2-11> | <4-2> |
| EC-623 | <W-1> | <1-5> | <2-11> | <4-3> |
| EC-624 | <W-1> | <1-5> | <2-11> | <4-4> |
| EC-625 | <W-1> | <1-5> | <2-11> | <4-5> |
| EC-626 | <W-1> | <1-5> | <2-11> | <4-6> |
| EC-627 | <W-1> | <1-5> | <2-11> | <4-7> |
| EC-628 | <W-1> | <1-5> | <2-11> | <4-8> |
| EC-629 | <W-1> | <1-5> | <2-11> | <4-9 |
| EC-630 | <W-1> | <1-5> | <2-11> | <4-10> |
| EC-631 | <W-1> | <1-5> | <2-12> | <4-1> |
| EC-632 | <W-1> | <1-5> | <2-12> | <4-2> |
| EC-633 | <W-1> | <1-5> | <2-12> | <4-3> |
| EC-634 | <W-1> | <1-5> | <2-12> | <4-4> |
| EC-635 | <W-1> | <1-5> | <2-12> | <4-5> |
| EC-636 | <W-1> | <1-5> | <2-12> | <4-6> |
| EC-637 | <W-1> | <1-5> | <2-12> | <4-7> |
| EC-638 | <W-1> | <1-5> | <2-12> | <4-8> |
| EC-639 | <W-1> | <1-5> | <2-12> | <4-9 |
| EC-640 | <W-1> | <1-5> | <2-12> | <4-10> |
| EC-641 | <W-1> | <1-5> | <2-13> | <4-1> |
| EC-642 | <W-1> | <1-5> | <2-13> | <4-2> |
| EC-643 | <W-1> | <1-5> | <2-13> | <4-3> |
| EC-644 | <W-1> | <1-5> | <2-13> | <4-4> |
| EC-645 | <W-1> | <1-5> | <2-13> | <4-5> |
| EC-646 | <W-1> | <1-5> | <2-13> | <4-6> |
| EC-647 | <W-1> | <1-5> | <2-13> | <4-7> |
| EC-648 | <W-1> | <1-5> | <2-13> | <4-8> |
| EC-649 | <W-1> | <1-5> | <2-13> | <4-9 |
| EC-650 | <W-1> | <1-5> | <2-13> | <4-10> |
| EC-651 | <W-1> | <1-6> | <2-1> | <4-1> |
| EC-652 | <W-1> | <1-6> | <2-1> | <4-2> |
| EC-653 | <W-1> | <1-6> | <2-1> | <4-3> |
| EC-654 | <W-1> | <1-6> | <2-1> | <4-4> |
| EC-655 | <W-1> | <1-6> | <2-1> | <4-5> |
| EC-656 | <W-1> | <1-6> | <2-1> | <4-6> |
| EC-657 | <W-1> | <1-6> | <2-1> | <4-7> |
| EC-658 | <W-1> | <1-6> | <2-1> | <4-8> |
| EC-659 | <W-1> | <1-6> | <2-1> | <4-9 |
| EC-660 | <W-1> | <1-6> | <2-1> | <4-10> |
| EC-661 | <W-1> | <1-6> | <2-2> | <4-1> |
| EC-662 | <W-1> | <1-6> | <2-2> | <4-2> |
| EC-663 | <W-1> | <1-6> | <2-2> | <4-3> |
| EC-664 | <W-1> | <1-6> | <2-2> | <4-4> |
| EC-665 | <W-1> | <1-6> | <2-2> | <4-5> |
| EC-666 | <W-1> | <1-6> | <2-2> | <4-6> |
| EC-667 | <W-1> | <1-6> | <2-2> | <4-7> |
| EC-668 | <W-1> | <1-6> | <2-2> | <4-8> |
| EC-669 | <W-1> | <1-6> | <2-2> | <4-9 |
| EC-670 | <W-1> | <1-6> | <2-2> | <4-10> |
| EC-671 | <W-1> | <1-6> | <2-3> | <4-1> |
| EC-672 | <W-1> | <1-6> | <2-3> | <4-2> |
| EC-673 | <W-1> | <1-6> | <2-3> | <4-3> |
| EC-674 | <W-1> | <1-6> | <2-3> | <4-4> |
| EC-675 | <W-1> | <1-6> | <2-3> | <4-5> |
| EC-676 | <W-1> | <1-6> | <2-3> | <4-6> |
| EC-677 | <W-1> | <1-6> | <2-3> | <4-7> |
| EC-678 | <W-1> | <1-6> | <2-3> | <4-8> |
| EC-679 | <W-1> | <1-6> | <2-3> | <4-9 |
| EC-680 | <W-1> | <1-6> | <2-3> | <4-10> |
| EC-681 | <W-1> | <1-6> | <2-4> | <4-1> |
| EC-682 | <W-1> | <1-6> | <2-4> | <4-2> |
| EC-683 | <W-1> | <1-6> | <2-4> | <4-3> |
| EC-684 | <W-1> | <1-6> | <2-4> | <4-4> |
| EC-685 | <W-1> | <1-6> | <2-4> | <4-5> |
| EC-686 | <W-1> | <1-6> | <2-4> | <4-6> |
| EC-687 | <W-1> | <1-6> | <2-4> | <4-7> |
| EC-688 | <W-1> | <1-6> | <2-4> | <4-8> |
| EC-689 | <W-1> | <1-6> | <2-4> | <4-9 |
| EC-690 | <W-1> | <1-6> | <2-4> | <4-10> |
| EC-691 | <W-1> | <1-6> | <2-5> | <4-1> |
| EC-692 | <W-1> | <1-6> | <2-5> | <4-2> |
| EC-693 | <W-1> | <1-6> | <2-5> | <4-3> |
| EC-694 | <W-1> | <1-6> | <2-5> | <4-4> |
| EC-695 | <W-1> | <1-6> | <2-5> | <4-5> |
| EC-696 | <W-1> | <1-6> | <2-5> | <4-6> |
| EC-697 | <W-1> | <1-6> | <2-5> | <4-7> |
| EC-698 | <W-1> | <1-6> | <2-5> | <4-8> |
| EC-699 | <W-1> | <1-6> | <2-5> | <4-9 |
| EC-700 | <W-1> | <1-6> | <2-5> | <4-10> |
| EC-701 | <W-1> | <1-6> | <2-6> | <4-1> |
| EC-702 | <W-1> | <1-6> | <2-6> | <4-2> |
| EC-703 | <W-1> | <1-6> | <2-6> | <4-3> |
| EC-704 | <W-1> | <1-6> | <2-6> | <4-4> |
| EC-705 | <W-1> | <1-6> | <2-6> | <4-5> |
| EC-706 | <W-1> | <1-6> | <2-6> | <4-6> |
| EC-707 | <W-1> | <1-6> | <2-6> | <4-7> |
| EC-708 | <W-1> | <1-6> | <2-6> | <4-8> |
| EC-709 | <W-1> | <1-6> | <2-6> | <4-9 |
| EC-710 | <W-1> | <1-6> | <2-6> | <4-10> |
| EC-711 | <W-1> | <1-6> | <2-7> | <4-1> |
| EC-712 | <W-1> | <1-6> | <2-7> | <4-2> |
| EC-713 | <W-1> | <1-6> | <2-7> | <4-3> |
| EC-714 | <W-1> | <1-6> | <2-7> | <4-4> |
| EC-715 | <W-1> | <1-6> | <2-7> | <4-5> |
| EC-716 | <W-1> | <1-6> | <2-7> | <4-6> |
| EC-717 | <W-1> | <1-6> | <2-7> | <4-7> |
| EC-718 | <W-1> | <1-6> | <2-7> | <4-8> |
| EC-719 | <W-1> | <1-6> | <2-7> | <4-9 |
| EC-720 | <W-1> | <1-6> | <2-7> | <4-10> |
| EC-721 | <W-1> | <1-6> | <2-8> | <4-1> |
| EC-722 | <W-1> | <1-6> | <2-8> | <4-2> |
| EC-723 | <W-1> | <1-6> | <2-8> | <4-3> |
| EC-724 | <W-1> | <1-6> | <2-8> | <4-4> |
| EC-725 | <W-1> | <1-6> | <2-8> | <4-5> |
| EC-726 | <W-1> | <1-6> | <2-8> | <4-6> |
| EC-727 | <W-1> | <1-6> | <2-8> | <4-7> |
| EC-728 | <W-1> | <1-6> | <2-8> | <4-8> |
| EC-729 | <W-1> | <1-6> | <2-8> | <4-9 |
| EC-730 | <W-1> | <1-6> | <2-8> | <4-10> |
| EC-731 | <W-1> | <1-6> | <2-9> | <4-1> |
| EC-732 | <W-1> | <1-6> | <2-9> | <4-2> |
| EC-733 | <W-1> | <1-6> | <2-9> | <4-3> |
| EC-734 | <W-1> | <1-6> | <2-9> | <4-4> |
| EC-735 | <W-1> | <1-6> | <2-9> | <4-5> |
| EC-736 | <W-1> | <1-6> | <2-9> | <4-6> |
| EC-737 | <W-1> | <1-6> | <2-9> | <4-7> |
| EC-738 | <W-1> | <1-6> | <2-9> | <4-8> |
| EC-739 | <W-1> | <1-6> | <2-9> | <4-9 |
| EC-740 | <W-1> | <1-6> | <2-9> | <4-10> |
| EC-741 | <W-1> | <1-6> | <2-10> | <4-1> |
| EC-742 | <W-1> | <1-6> | <2-10> | <4-2> |
| EC-743 | <W-1> | <1-6> | <2-10> | <4-3> |
| EC-744 | <W-1> | <1-6> | <2-10> | <4-4> |
| EC-745 | <W-1> | <1-6> | <2-10> | <4-5> |
| EC-746 | <W-1> | <1-6> | <2-10> | <4-6> |
| EC-747 | <W-1> | <1-6> | <2-10> | <4-7> |
| EC-748 | <W-1> | <1-6> | <2-10> | <4-8> |
| EC-749 | <W-1> | <1-6> | <2-10> | <4-9 |
| EC-750 | <W-1> | <1-6> | <2-10> | <4-10> |
| EC-751 | <W-1> | <1-6> | <2-11> | <4-1> |
| EC-752 | <W-1> | <1-6> | <2-11> | <4-2> |
| EC-753 | <W-1> | <1-6> | <2-11> | <4-3> |
| EC-754 | <W-1> | <1-6> | <2-11> | <4-4> |
| EC-755 | <W-1> | <1-6> | <2-11> | <4-5> |
| EC-756 | <W-1> | <1-6> | <2-11> | <4-6> |
| EC-757 | <W-1> | <1-6> | <2-11> | <4-7> |
| EC-758 | <W-1> | <1-6> | <2-11> | <4-8> |
| EC-759 | <W-1> | <1-6> | <2-11> | <4-9 |
| EC-760 | <W-1> | <1-6> | <2-11> | <4-10> |
| EC-761 | <W-1> | <1-6> | <2-12> | <4-1> |
| EC-762 | <W-1> | <1-6> | <2-12> | <4-2> |
| EC-763 | <W-1> | <1-6> | <2-12> | <4-3> |
| EC-764 | <W-1> | <1-6> | <2-12> | <4-4> |
| EC-765 | <W-1> | <1-6> | <2-12> | <4-5> |
| EC-766 | <W-1> | <1-6> | <2-12> | <4-6> |
| EC-767 | <W-1> | <1-6> | <2-12> | <4-7> |
| EC-768 | <W-1> | <1-6> | <2-12> | <4-8> |
| EC-769 | <W-1> | <1-6> | <2-12> | <4-9 |
| EC-770 | <W-1> | <1-6> | <2-12> | <4-10> |
| EC-771 | <W-1> | <1-6> | <2-13> | <4-1> |
| EC-772 | <W-1> | <1-6> | <2-13> | <4-2> |
| EC-773 | <W-1> | <1-6> | <2-13> | <4-3> |
| EC-774 | <W-1> | <1-6> | <2-13> | <4-4> |
| EC-775 | <W-1> | <1-6> | <2-13> | <4-5> |
| EC-776 | <W-1> | <1-6> | <2-13> | <4-6> |
| EC-777 | <W-1> | <1-6> | <2-13> | <4-7> |
| EC-778 | <W-1> | <1-6> | <2-13> | <4-8> |
| EC-779 | <W-1> | <1-6> | <2-13> | <4-9 |
| EC-780 | <W-1> | <1-6> | <2-13> | <4-10> |
| EC-781 | <W-1> | <1-7> | <2-1> | <4-1> |
| EC-782 | <W-1> | <1-7> | <2-1> | <4-2> |
| EC-783 | <W-1> | <1-7> | <2-1> | <4-3> |
| EC-784 | <W-1> | <1-7> | <2-1> | <4-4> |
| EC-785 | <W-1> | <1-7> | <2-1> | <4-5> |
| EC-786 | <W-1> | <1-7> | <2-1> | <4-6> |
| EC-787 | <W-1> | <1-7> | <2-1> | <4-7> |
| EC-788 | <W-1> | <1-7> | <2-1> | <4-8> |
| EC-789 | <W-1> | <1-7> | <2-1> | <4-9 |
| EC-790 | <W-1> | <1-7> | <2-1> | <4-10> |
| EC-791 | <W-1> | <1-7> | <2-2> | <4-1> |
| EC-792 | <W-1> | <1-7> | <2-2> | <4-2> |
| EC-793 | <W-1> | <1-7> | <2-2> | <4-3> |
| EC-794 | <W-1> | <1-7> | <2-2> | <4-4> |
| EC-795 | <W-1> | <1-7> | <2-2> | <4-5> |
| EC-796 | <W-1> | <1-7> | <2-2> | <4-6> |
| EC-797 | <W-1> | <1-7> | <2-2> | <4-7> |
| EC-798 | <W-1> | <1-7> | <2-2> | <4-8> |
| EC-799 | <W-1> | <1-7> | <2-2> | <4-9 |
| EC-800 | <W-1> | <1-7> | <2-2> | <4-10> |
| EC-801 | <W-1> | <1-7> | <2-3> | <4-1> |
| EC-802 | <W-1> | <1-7> | <2-3> | <4-2> |
| EC-803 | <W-1> | <1-7> | <2-3> | <4-3> |
| EC-804 | <W-1> | <1-7> | <2-3> | <4-4> |
| EC-805 | <W-1> | <1-7> | <2-3> | <4-5> |
| EC-806 | <W-1> | <1-7> | <2-3> | <4-6> |
| EC-807 | <W-1> | <1-7> | <2-3> | <4-7> |
| EC-808 | <W-1> | <1-7> | <2-3> | <4-8> |
| EC-809 | <W-1> | <1-7> | <2-3> | <4-9 |
| EC-810 | <W-1> | <1-7> | <2-3> | <4-10> |
| EC-811 | <W-1> | <1-7> | <2-4> | <4-1> |
| EC-812 | <W-1> | <1-7> | <2-4> | <4-2> |
| EC-813 | <W-1> | <1-7> | <2-4> | <4-3> |
| EC-814 | <W-1> | <1-7> | <2-4> | <4-4> |
| EC-815 | <W-1> | <1-7> | <2-4> | <4-5> |
| EC-816 | <W-1> | <1-7> | <2-4> | <4-6> |
| EC-817 | <W-1> | <1-7> | <2-4> | <4-7> |
| EC-818 | <W-1> | <1-7> | <2-4> | <4-8> |
| EC-819 | <W-1> | <1-7> | <2-4> | <4-9 |
| EC-820 | <W-1> | <1-7> | <2-4> | <4-10> |
| EC-821 | <W-1> | <1-7> | <2-5> | <4-1> |
| EC-822 | <W-1> | <1-7> | <2-5> | <4-2> |
| EC-823 | <W-1> | <1-7> | <2-5> | <4-3> |
| EC-824 | <W-1> | <1-7> | <2-5> | <4-4> |
| EC-825 | <W-1> | <1-7> | <2-5> | <4-5> |
| EC-826 | <W-1> | <1-7> | <2-5> | <4-6> |
| EC-827 | <W-1> | <1-7> | <2-5> | <4-7> |
| EC-828 | <W-1> | <1-7> | <2-5> | <4-8> |
| EC-829 | <W-1> | <1-7> | <2-5> | <4-9 |
| EC-830 | <W-1> | <1-7> | <2-5> | <4-10> |
| EC-831 | <W-1> | <1-7> | <2-6> | <4-1> |
| EC-832 | <W-1> | <1-7> | <2-6> | <4-2> |
| EC-833 | <W-1> | <1-7> | <2-6> | <4-3> |
| EC-834 | <W-1> | <1-7> | <2-6> | <4-4> |
| EC-835 | <W-1> | <1-7> | <2-6> | <4-5> |
| EC-836 | <W-1> | <1-7> | <2-6> | <4-6> |
| EC-837 | <W-1> | <1-7> | <2-6> | <4-7> |
| EC-838 | <W-1> | <1-7> | <2-6> | <4-8> |
| EC-839 | <W-1> | <1-7> | <2-6> | <4-9 |
| EC-840 | <W-1> | <1-7> | <2-6> | <4-10> |
| EC-841 | <W-1> | <1-7> | <2-7> | <4-1> |
| EC-842 | <W-1> | <1-7> | <2-7> | <4-2> |
| EC-843 | <W-1> | <1-7> | <2-7> | <4-3> |
| EC-844 | <W-1> | <1-7> | <2-7> | <4-4> |
| EC-845 | <W-1> | <1-7> | <2-7> | <4-5> |
| EC-846 | <W-1> | <1-7> | <2-7> | <4-6> |
| EC-847 | <W-1> | <1-7> | <2-7> | <4-7> |
| EC-848 | <W-1> | <1-7> | <2-7> | <4-8> |
| EC-849 | <W-1> | <1-7> | <2-7> | <4-9 |
| EC-850 | <W-1> | <1-7> | <2-7> | <4-10> |
| EC-851 | <W-1> | <1-7> | <2-8> | <4-1> |
| EC-852 | <W-1> | <1-7> | <2-8> | <4-2> |
| EC-853 | <W-1> | <1-7> | <2-8> | <4-3> |
| EC-854 | <W-1> | <1-7> | <2-8> | <4-4> |
| EC-855 | <W-1> | <1-7> | <2-8> | <4-5> |
| EC-856 | <W-1> | <1-7> | <2-8> | <4-6> |
| EC-857 | <W-1> | <1-7> | <2-8> | <4-7> |
| EC-858 | <W-1> | <1-7> | <2-8> | <4-8> |
| EC-859 | <W-1> | <1-7> | <2-8> | <4-9 |
| EC-860 | <W-1> | <1-7> | <2-8> | <4-10> |
| EC-861 | <W-1> | <1-7> | <2-9> | <4-1> |
| EC-862 | <W-1> | <1-7> | <2-9> | <4-2> |
| EC-863 | <W-1> | <1-7> | <2-9> | <4-3> |
| EC-864 | <W-1> | <1-7> | <2-9> | <4-4> |
| EC-865 | <W-1> | <1-7> | <2-9> | <4-5> |
| EC-866 | <W-1> | <1-7> | <2-9> | <4-6> |
| EC-867 | <W-1> | <1-7> | <2-9> | <4-7> |
| EC-868 | <W-1> | <1-7> | <2-9> | <4-8> |
| EC-869 | <W-1> | <1-7> | <2-9> | <4-9 |
| EC-870 | <W-1> | <1-7> | <2-9> | <4-10> |
| EC-871 | <W-1> | <1-7> | <2-10> | <4-1> |
| EC-872 | <W-1> | <1-7> | <2-10> | <4-2> |
| EC-873 | <W-1> | <1-7> | <2-10> | <4-3> |
| EC-874 | <W-1> | <1-7> | <2-10> | <4-4> |
| EC-875 | <W-1> | <1-7> | <2-10> | <4-5> |
| EC-876 | <W-1> | <1-7> | <2-10> | <4-6> |
| EC-877 | <W-1> | <1-7> | <2-10> | <4-7> |
| EC-878 | <W-1> | <1-7> | <2-10> | <4-8> |
| EC-879 | <W-1> | <1-7> | <2-10> | <4-9 |
| EC-880 | <W-1> | <1-7> | <2-10> | <4-10> |
| EC-881 | <W-1> | <1-7> | <2-11> | <4-1> |
| EC-882 | <W-1> | <1-7> | <2-11> | <4-2> |
| EC-883 | <W-1> | <1-7> | <2-11> | <4-3> |
| EC-884 | <W-1> | <1-7> | <2-11> | <4-4> |
| EC-885 | <W-1> | <1-7> | <2-11> | <4-5> |
| EC-886 | <W-1> | <1-7> | <2-11> | <4-6> |
| EC-887 | <W-1> | <1-7> | <2-11> | <4-7> |
| EC-888 | <W-1> | <1-7> | <2-11> | <4-8> |
| EC-889 | <W-1> | <1-7> | <2-11> | <4-9 |
| EC-890 | <W-1> | <1-7> | <2-11> | <4-10> |
| EC-891 | <W-1> | <1-7> | <2-12> | <4-1> |
| EC-892 | <W-1> | <1-7> | <2-12> | <4-2> |
| EC-893 | <W-1> | <1-7> | <2-12> | <4-3> |
| EC-894 | <W-1> | <1-7> | <2-12> | <4-4> |
| EC-895 | <W-1> | <1-7> | <2-12> | <4-5> |
| EC-896 | <W-1> | <1-7> | <2-12> | <4-6> |
| EC-897 | <W-1> | <1-7> | <2-12> | <4-7> |
| EC-898 | <W-1> | <1-7> | <2-12> | <4-8> |
| EC-899 | <W-1> | <1-7> | <2-12> | <4-9 |
| EC-900 | <W-1> | <1-7> | <2-12> | <4-10> |
| EC-901 | <W-1> | <1-7> | <2-13> | <4-1> |
| EC-902 | <W-1> | <1-7> | <2-13> | <4-2> |
| EC-903 | <W-1> | <1-7> | <2-13> | <4-3> |
| EC-904 | <W-1> | <1-7> | <2-13> | <4-4> |
| EC-905 | <W-1> | <1-7> | <2-13> | <4-5> |
| EC-906 | <W-1> | <1-7> | <2-13> | <4-6> |
| EC-907 | <W-1> | <1-7> | <2-13> | <4-7> |
| EC-908 | <W-1> | <1-7> | <2-13> | <4-8> |
| EC-909 | <W-1> | <1-7> | <2-13> | <4-9 |
| EC-910 | <W-1> | <1-7> | <2-13> | <4-10> |
| EC-911 | <W-1> | <1-8> | <2-1> | <4-1> |
| EC-912 | <W-1> | <1-8> | <2-1> | <4-2> |
| EC-913 | <W-1> | <1-8> | <2-1> | <4-3> |
| EC-914 | <W-1> | <1-8> | <2-1> | <4-4> |
| EC-915 | <W-1> | <1-8> | <2-1> | <4-5> |
| EC-916 | <W-1> | <1-8> | <2-1> | <4-6> |
| EC-917 | <W-1> | <1-8> | <2-1> | <4-7> |
| EC-918 | <W-1> | <1-8> | <2-1> | <4-8> |
| EC-919 | <W-1> | <1-8> | <2-1> | <4-9 |
| EC-920 | <W-1> | <1-8> | <2-1> | <4-10> |
| EC-921 | <W-1> | <1-8> | <2-2> | <4-1> |
| EC-922 | <W-1> | <1-8> | <2-2> | <4-2> |
| EC-923 | <W-1> | <1-8> | <2-2> | <4-3> |
| EC-924 | <W-1> | <1-8> | <2-2> | <4-4> |
| EC-925 | <W-1> | <1-8> | <2-2> | <4-5> |
| EC-926 | <W-1> | <1-8> | <2-2> | <4-6> |
| EC-927 | <W-1> | <1-8> | <2-2> | <4-7> |
| EC-928 | <W-1> | <1-8> | <2-2> | <4-8> |
| EC-929 | <W-1> | <1-8> | <2-2> | <4-9 |
| EC-930 | <W-1> | <1-8> | <2-2> | <4-10> |
| EC-931 | <W-1> | <1-8> | <2-3> | <4-1> |
| EC-932 | <W-1> | <1-8> | <2-3> | <4-2> |
| EC-933 | <W-1> | <1-8> | <2-3> | <4-3> |
| EC-934 | <W-1> | <1-8> | <2-3> | <4-4> |
| EC-935 | <W-1> | <1-8> | <2-3> | <4-5> |
| EC-936 | <W-1> | <1-8> | <2-3> | <4-6> |
| EC-937 | <W-1> | <1-8> | <2-3> | <4-7> |
| EC-938 | <W-1> | <1-8> | <2-3> | <4-8> |
| EC-939 | <W-1> | <1-8> | <2-3> | <4-9 |
| EC-940 | <W-1> | <1-8> | <2-3> | <4-10> |
| EC-941 | <W-1> | <1-8> | <2-4> | <4-1> |
| EC-942 | <W-1> | <1-8> | <2-4> | <4-2> |
| EC-943 | <W-1> | <1-8> | <2-4> | <4-3> |
| EC-944 | <W-1> | <1-8> | <2-4> | <4-4> |
| EC-945 | <W-1> | <1-8> | <2-4> | <4-5> |
| EC-946 | <W-1> | <1-8> | <2-4> | <4-6> |
| EC-947 | <W-1> | <1-8> | <2-4> | <4-7> |
| EC-948 | <W-1> | <1-8> | <2-4> | <4-8> |
| EC-949 | <W-1> | <1-8> | <2-4> | <4-9 |
| EC-950 | <W-1> | <1-8> | <2-4> | <4-10> |
| EC-951 | <W-1> | <1-8> | <2-5> | <4-1> |
| EC-952 | <W-1> | <1-8> | <2-5> | <4-2> |
| EC-953 | <W-1> | <1-8> | <2-5> | <4-3> |
| EC-954 | <W-1> | <1-8> | <2-5> | <4-4> |
| EC-955 | <W-1> | <1-8> | <2-5> | <4-5> |
| EC-956 | <W-1> | <1-8> | <2-5> | <4-6> |
| EC-957 | <W-1> | <1-8> | <2-5> | <4-7> |
| EC-958 | <W-1> | <1-8> | <2-5> | <4-8> |
| EC-959 | <W-1> | <1-8> | <2-5> | <4-9 |
| EC-960 | <W-1> | <1-8> | <2-5> | <4-10> |
| EC-961 | <W-1> | <1-8> | <2-6> | <4-1> |
| EC-962 | <W-1> | <1-8> | <2-6> | <4-2> |
| EC-963 | <W-1> | <1-8> | <2-6> | <4-3> |
| EC-964 | <W-1> | <1-8> | <2-6> | <4-4> |
| EC-965 | <W-1> | <1-8> | <2-6> | <4-5> |
| EC-966 | <W-1> | <1-8> | <2-6> | <4-6> |
| EC-967 | <W-1> | <1-8> | <2-6> | <4-7> |
| EC-968 | <W-1> | <1-8> | <2-6> | <4-8> |
| EC-969 | <W-1> | <1-8> | <2-6> | <4-9 |
| EC-970 | <W-1> | <1-8> | <2-6> | <4-10> |
| EC-971 | <W-1> | <1-8> | <2-7> | <4-1> |
| EC-972 | <W-1> | <1-8> | <2-7> | <4-2> |
| EC-973 | <W-1> | <1-8> | <2-7> | <4-3> |
| EC-974 | <W-1> | <1-8> | <2-7> | <4-4> |
| EC-975 | <W-1> | <1-8> | <2-7> | <4-5> |
| EC-976 | <W-1> | <1-8> | <2-7> | <4-6> |
| EC-977 | <W-1> | <1-8> | <2-7> | <4-7> |
| EC-978 | <W-1> | <1-8> | <2-7> | <4-8> |
| EC-979 | <W-1> | <1-8> | <2-7> | <4-9 |
| EC-980 | <W-1> | <1-8> | <2-7> | <4-10> |
| EC-981 | <W-1> | <1-8> | <2-8> | <4-1> |
| EC-982 | <W-1> | <1-8> | <2-8> | <4-2> |
| EC-983 | <W-1> | <1-8> | <2-8> | <4-3> |
| EC-984 | <W-1> | <1-8> | <2-8> | <4-4> |
| EC-985 | <W-1> | <1-8> | <2-8> | <4-5> |
| EC-986 | <W-1> | <1-8> | <2-8> | <4-6> |
| EC-987 | <W-1> | <1-8> | <2-8> | <4-7> |
| EC-988 | <W-1> | <1-8> | <2-8> | <4-8> |
| EC-989 | <W-1> | <1-8> | <2-8> | <4-9 |
| EC-990 | <W-1> | <1-8> | <2-8> | <4-10> |
| EC-991 | <W-1> | <1-8> | <2-9> | <4-1> |
| EC-992 | <W-1> | <1-8> | <2-9> | <4-2> |
| EC-993 | <W-1> | <1-8> | <2-9> | <4-3> |
| EC-994 | <W-1> | <1-8> | <2-9> | <4-4> |
| EC-995 | <W-1> | <1-8> | <2-9> | <4-5> |
| EC-996 | <W-1> | <1-8> | <2-9> | <4-6> |
| EC-997 | <W-1> | <1-8> | <2-9> | <4-7> |
| EC-998 | <W-1> | <1-8> | <2-9> | <4-8> |
| EC-999 | <W-1> | <1-8> | <2-9> | <4-9 |
| EC-1000 | <W-1> | <1-8> | <2-9> | <4-10> |
| EC-1001 | <W-1> | <1-8> | <2-10> | <4-1> |
| EC-1002 | <W-1> | <1-8> | <2-10> | <4-2> |
| EC-1003 | <W-1> | <1-8> | <2-10> | <4-3> |
| EC-1004 | <W-1> | <1-8> | <2-10> | <4-4> |
| EC-1005 | <W-1> | <1-8> | <2-10> | <4-5> |
| EC-1006 | <W-1> | <1-8> | <2-10> | <4-6> |
| EC-1007 | <W-1> | <1-8> | <2-10> | <4-7> |
| EC-1008 | <W-1> | <1-8> | <2-10> | <4-8> |
| EC-1009 | <W-1> | <1-8> | <2-10> | <4-9 |
| EC-1010 | <W-1> | <1-8> | <2-10> | <4-10> |
| EC-1011 | <W-1> | <1-8> | <2-11> | <4-1> |
| EC-1012 | <W-1> | <1-8> | <2-11> | <4-2> |
| EC-1013 | <W-1> | <1-8> | <2-11> | <4-3> |
| EC-1014 | <W-1> | <1-8> | <2-11> | <4-4> |
| EC-1015 | <W-1> | <1-8> | <2-11> | <4-5> |
| EC-1016 | <W-1> | <1-8> | <2-11> | <4-6> |
| EC-1017 | <W-1> | <1-8> | <2-11> | <4-7> |
| EC-1018 | <W-1> | <1-8> | <2-11> | <4-8> |
| EC-1019 | <W-1> | <1-8> | <2-11> | <4-9 |
| EC-1020 | <W-1> | <1-8> | <2-11> | <4-10> |
| EC-1021 | <W-1> | <1-8> | <2-12> | <4-1> |
| EC-1022 | <W-1> | <1-8> | <2-12> | <4-2> |
| EC-1023 | <W-1> | <1-8> | <2-12> | <4-3> |
| EC-1024 | <W-1> | <1-8> | <2-12> | <4-4> |
| EC-1025 | <W-1> | <1-8> | <2-12> | <4-5> |
| EC-1026 | <W-1> | <1-8> | <2-12> | <4-6> |
| EC-1027 | <W-1> | <1-8> | <2-12> | <4-7> |
| EC-1028 | <W-1> | <1-8> | <2-12> | <4-8> |
| EC-1029 | <W-1> | <1-8> | <2-12> | <4-9 |
| EC-1030 | <W-1> | <1-8> | <2-12> | <4-10> |
| EC-1031 | <W-1> | <1-8> | <2-13> | <4-1> |
| EC-1032 | <W-1> | <1-8> | <2-13> | <4-2> |
| EC-1033 | <W-1> | <1-8> | <2-13> | <4-3> |
| EC-1034 | <W-1> | <1-8> | <2-13> | <4-4> |
| EC-1035 | <W-1> | <1-8> | <2-13> | <4-5> |
| EC-1036 | <W-1> | <1-8> | <2-13> | <4-6> |
| EC-1037 | <W-1> | <1-8> | <2-13> | <4-7> |
| EC-1038 | <W-1> | <1-8> | <2-13> | <4-8> |
| EC-1039 | <W-1> | <1-8> | <2-13> | <4-9 |
| EC-1040 | <W-1> | <1-8> | <2-13> | <4-10> |
| EC-1041 | <W-1> | <1-9> | <2-1> | <4-1> |
| EC-1042 | <W-1> | <1-9> | <2-1> | <4-2> |
| EC-1043 | <W-1> | <1-9> | <2-1> | <4-3> |
| EC-1044 | <W-1> | <1-9> | <2-1> | <4-4> |
| EC-1045 | <W-1> | <1-9> | <2-1> | <4-5> |
| EC-1046 | <W-1> | <1-9> | <2-1> | <4-6> |
| EC-1047 | <W-1> | <1-9> | <2-1> | <4-7> |
| EC-1048 | <W-1> | <1-9> | <2-1> | <4-8> |
| EC-1049 | <W-1> | <1-9> | <2-1> | <4-9 |
| EC-1050 | <W-1> | <1-9> | <2-1> | <4-10> |
| EC-1051 | <W-1> | <1-9> | <2-2> | <4-1> |
| EC-1052 | <W-1> | <1-9> | <2-2> | <4-2> |
| EC-1053 | <W-1> | <1-9> | <2-2> | <4-3> |
| EC-1054 | <W-1> | <1-9> | <2-2> | <4-4> |
| EC-1055 | <W-1> | <1-9> | <2-2> | <4-5> |
| EC-1056 | <W-1> | <1-9> | <2-2> | <4-6> |
| EC-1057 | <W-1> | <1-9> | <2-2> | <4-7> |
| EC-1058 | <W-1> | <1-9> | <2-2> | <4-8> |
| EC-1059 | <W-1> | <1-9> | <2-2> | <4-9 |
| EC-1060 | <W-1> | <1-9> | <2-2> | <4-10> |
| EC-1061 | <W-1> | <1-9> | <2-3> | <4-1> |
| EC-1062 | <W-1> | <1-9> | <2-3> | <4-2> |
| EC-1063 | <W-1> | <1-9> | <2-3> | <4-3> |
| EC-1064 | <W-1> | <1-9> | <2-3> | <4-4> |
| EC-1065 | <W-1> | <1-9> | <2-3> | <4-5> |
| EC-1066 | <W-1> | <1-9> | <2-3> | <4-6> |
| EC-1067 | <W-1> | <1-9> | <2-3> | <4-7> |
| EC-1068 | <W-1> | <1-9> | <2-3> | <4-8> |
| EC-1069 | <W-1> | <1-9> | <2-3> | <4-9 |
| EC-1070 | <W-1> | <1-9> | <2-3> | <4-10> |
| EC-1071 | <W-1> | <1-9> | <2-4> | <4-1> |
| EC-1072 | <W-1> | <1-9> | <2-4> | <4-2> |
| EC-1073 | <W-1> | <1-9> | <2-4> | <4-3> |
| EC-1074 | <W-1> | <1-9> | <2-4> | <4-4> |
| EC-1075 | <W-1> | <1-9> | <2-4> | <4-5> |
| EC-1076 | <W-1> | <1-9> | <2-4> | <4-6> |
| EC-1077 | <W-1> | <1-9> | <2-4> | <4-7> |
| EC-1078 | <W-1> | <1-9> | <2-4> | <4-8> |
| EC-1079 | <W-1> | <1-9> | <2-4> | <4-9 |
| EC-1080 | <W-1> | <1-9> | <2-4> | <4-10> |
| EC-1081 | <W-1> | <1-9> | <2-5> | <4-1> |
| EC-1082 | <W-1> | <1-9> | <2-5> | <4-2> |
| EC-1083 | <W-1> | <1-9> | <2-5> | <4-3> |
| EC-1084 | <W-1> | <1-9> | <2-5> | <4-4> |
| EC-1085 | <W-1> | <1-9> | <2-5> | <4-5> |
| EC-1086 | <W-1> | <1-9> | <2-5> | <4-6> |
| EC-1087 | <W-1> | <1-9> | <2-5> | <4-7> |
| EC-1088 | <W-1> | <1-9> | <2-5> | <4-8> |
| EC-1089 | <W-1> | <1-9> | <2-5> | <4-9 |
| EC-1090 | <W-1> | <1-9> | <2-5> | <4-10> |
| EC-1091 | <W-1> | <1-9> | <2-6> | <4-1> |
| EC-1092 | <W-1> | <1-9> | <2-6> | <4-2> |
| EC-1093 | <W-1> | <1-9> | <2-6> | <4-3> |
| EC-1094 | <W-1> | <1-9> | <2-6> | <4-4> |
| EC-1095 | <W-1> | <1-9> | <2-6> | <4-5> |
| EC-1096 | <W-1> | <1-9> | <2-6> | <4-6> |
| EC-1097 | <W-1> | <1-9> | <2-6> | <4-7> |
| EC-1098 | <W-1> | <1-9> | <2-6> | <4-8> |
| EC-1099 | <W-1> | <1-9> | <2-6> | <4-9 |
| EC-1100 | <W-1> | <1-9> | <2-6> | <4-10> |
| EC-1101 | <W-1> | <1-9> | <2-7> | <4-1> |
| EC-1102 | <W-1> | <1-9> | <2-7> | <4-2> |
| EC-1103 | <W-1> | <1-9> | <2-7> | <4-3> |
| EC-1104 | <W-1> | <1-9> | <2-7> | <4-4> |
| EC-1105 | <W-1> | <1-9> | <2-7> | <4-5> |
| EC-1106 | <W-1> | <1-9> | <2-7> | <4-6> |
| EC-1107 | <W-1> | <1-9> | <2-7> | <4-7> |
| EC-1108 | <W-1> | <1-9> | <2-7> | <4-8> |
| EC-1109 | <W-1> | <1-9> | <2-7> | <4-9 |
| EC-1110 | <W-1> | <1-9> | <2-7> | <4-10> |
| EC-1111 | <W-1> | <1-9> | <2-8> | <4-1> |
| EC-1112 | <W-1> | <1-9> | <2-8> | <4-2> |
| EC-1113 | <W-1> | <1-9> | <2-8> | <4-3> |
| EC-1114 | <W-1> | <1-9> | <2-8> | <4-4> |
| EC-1115 | <W-1> | <1-9> | <2-8> | <4-5> |
| EC-1116 | <W-1> | <1-9> | <2-8> | <4-6> |
| EC-1117 | <W-1> | <1-9> | <2-8> | <4-7> |
| EC-1118 | <W-1> | <1-9> | <2-8> | <4-8> |
| EC-1119 | <W-1> | <1-9> | <2-8> | <4-9 |
| EC-1120 | <W-1> | <1-9> | <2-8> | <4-10> |
| EC-1121 | <W-1> | <1-9> | <2-9> | <4-1> |
| EC-1122 | <W-1> | <1-9> | <2-9> | <4-2> |
| EC-1123 | <W-1> | <1-9> | <2-9> | <4-3> |
| EC-1124 | <W-1> | <1-9> | <2-9> | <4-4> |
| EC-1125 | <W-1> | <1-9> | <2-9> | <4-5> |
| EC-1126 | <W-1> | <1-9> | <2-9> | <4-6> |
| EC-1127 | <W-1> | <1-9> | <2-9> | <4-7> |
| EC-1128 | <W-1> | <1-9> | <2-9> | <4-8> |
| EC-1129 | <W-1> | <1-9> | <2-9> | <4-9 |
| EC-1130 | <W-1> | <1-9> | <2-9> | <4-10> |
| EC-1131 | <W-1> | <1-9> | <2-10> | <4-1> |
| EC-1132 | <W-1> | <1-9> | <2-10> | <4-2> |
| EC-1133 | <W-1> | <1-9> | <2-10> | <4-3> |
| EC-1134 | <W-1> | <1-9> | <2-10> | <4-4> |
| EC-1135 | <W-1> | <1-9> | <2-10> | <4-5> |
| EC-1136 | <W-1> | <1-9> | <2-10> | <4-6> |
| EC-1137 | <W-1> | <1-9> | <2-10> | <4-7> |
| EC-1138 | <W-1> | <1-9> | <2-10> | <4-8> |
| EC-1139 | <W-1> | <1-9> | <2-10> | <4-9 |
| EC-1140 | <W-1> | <1-9> | <2-10> | <4-10> |
| EC-1141 | <W-1> | <1-9> | <2-11> | <4-1> |
| EC-1142 | <W-1> | <1-9> | <2-11> | <4-2> |
| EC-1143 | <W-1> | <1-9> | <2-11> | <4-3> |
| EC-1144 | <W-1> | <1-9> | <2-11> | <4-4> |
| EC-1145 | <W-1> | <1-9> | <2-11> | <4-5> |
| EC-1146 | <W-1> | <1-9> | <2-11> | <4-6> |
| EC-1147 | <W-1> | <1-9> | <2-11> | <4-7> |
| EC-1148 | <W-1> | <1-9> | <2-11> | <4-8> |
| EC-1149 | <W-1> | <1-9> | <2-11> | <4-9 |
| EC-1150 | <W-1> | <1-9> | <2-11> | <4-10> |
| EC-1151 | <W-1> | <1-9> | <2-12> | <4-1> |
| EC-1152 | <W-1> | <1-9> | <2-12> | <4-2> |
| EC-1153 | <W-1> | <1-9> | <2-12> | <4-3> |
| EC-1154 | <W-1> | <1-9> | <2-12> | <4-4> |
| EC-1155 | <W-1> | <1-9> | <2-12> | <4-5> |
| EC-1156 | <W-1> | <1-9> | <2-12> | <4-6> |
| EC-1157 | <W-1> | <1-9> | <2-12> | <4-7> |
| EC-1158 | <W-1> | <1-9> | <2-12> | <4-8> |
| EC-1159 | <W-1> | <1-9> | <2-12> | <4-9 |
| EC-1160 | <W-1> | <1-9> | <2-12> | <4-10> |
| EC-1161 | <W-1> | <1-9> | <2-13> | <4-1> |
| EC-1162 | <W-1> | <1-9> | <2-13> | <4-2> |
| EC-1163 | <W-1> | <1-9> | <2-13> | <4-3> |
| EC-1164 | <W-1> | <1-9> | <2-13> | <4-4> |
| EC-1165 | <W-1> | <1-9> | <2-13> | <4-5> |
| EC-1166 | <W-1> | <1-9> | <2-13> | <4-6> |
| EC-1167 | <W-1> | <1-9> | <2-13> | <4-7> |
| EC-1168 | <W-1> | <1-9> | <2-13> | <4-8> |
| EC-1169 | <W-1> | <1-9> | <2-13> | <4-9 |
| EC-1170 | <W-1> | <1-9> | <2-13> | <4-10> |

In the same way, the combinations EC-1171 to EC-2340 are part of the invention, wherein <W-2> is used instead of <W-1>.

In the same way, the combinations EC-2341 to EC-3510 are part of the invention, wherein <W-3> is used instead of <W-1>.

In the same way, the combinations EC-3511 to EC-4680 are part of the invention, wherein <W-2> is used instead of <W-1>.

In particular, with a view to their use, preference is given to the compounds of the formula (I) compiled in the tables below, and to the preferred compounds of formula II-1, II-2, II-3, II-4, II-5, II-6, II-7, and II-15. Each of the groups mentioned for the substituents in the tables are furthermore per se, independently of the combination in which they are mentioned, a particularly preferred aspect of the substituent in question. Further, each individual meaning of a substituent in the tables constitutes a particularly preferred embodiment of the substituents in question.

Table 1: Compounds of the formula (III-1) corresponding to the compounds of the formula II-1, in which the combination of R¹, R⁴ and T for a compound corresponds in each case to one line of Table A:

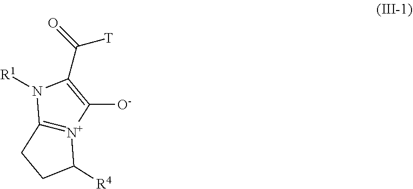

Table 2: Compounds of the formula (III-2) corresponding to the compounds of the formula II-2, in which the combination of R¹, R⁴ and T for a compound corresponds in each case to one line of Table A:

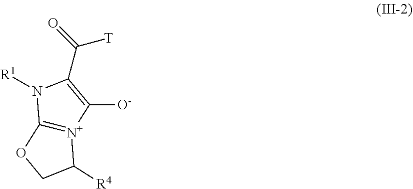

Table 3: Compounds of the formula (III-3) corresponding to the compounds of the formula II-3, in which the combination of R¹, R⁴ and T for a compound corresponds in each case to one line of Table A:

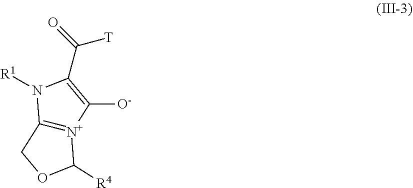

Table 4: Compounds of the formula (III-4) corresponding to the compounds of the formula II-4, in which the combination of R¹, R⁴ and T for a compound corresponds in each case to one line of Table A:

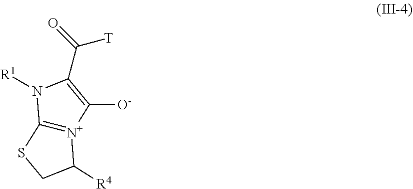

Table 5: Compounds of the formula (III-5) corresponding to the compounds of the formula II-5, in which the combination of R¹, R⁴ and T for a compound corresponds in each case to one line of Table A:

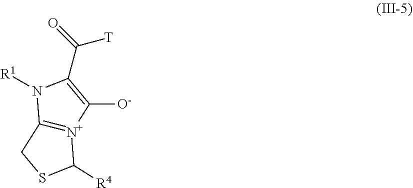

Table 6: Compounds of the formula (III-6) corresponding to the compounds of the formula II-6, in which the combination of R¹, R⁴ and T for a compound corresponds in each case to one line of Table A:

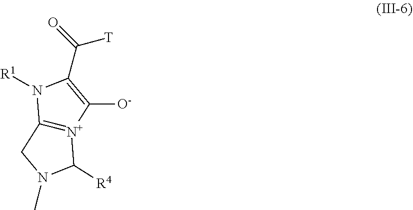

Table 7: Compounds of the formula (III-7) corresponding to the compounds of the formula II-7, in which the combination of R¹, R⁴ and T for a compound corresponds in each case to one line of Table A:

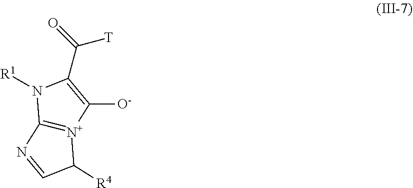

Table 8: Compounds of the formula (III-8) corresponding to the compounds of the formula II-8, in which the combination of R¹, R⁴ and T for a compound corresponds in each case to one line of Table A:

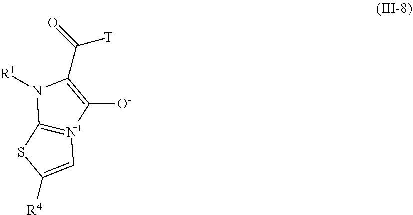

Table 9: Compounds of the formula (III-9) corresponding to the compounds of the formula II-9, in which the combination of R¹, R⁴ and T for a compound corresponds in each case to one line of Table A:

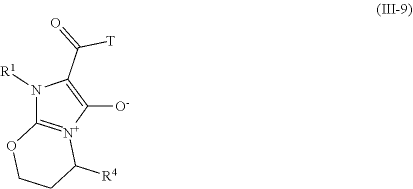

Table 10: Compounds of the formula (III-10) corresponding to this compounds of the formula II-10, in which the combination of R¹, R⁴ and T for a compound corresponds in each case to one line of Table A:

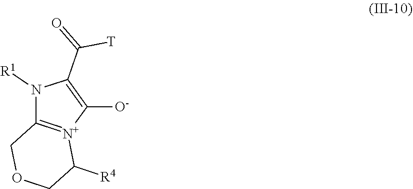

Table 11: Compounds of the formula (III-11) corresponding to the compounds of the formula II-15, in which the combination of R¹, R⁴ and T for a compound corresponds in each case to one line of Table A:

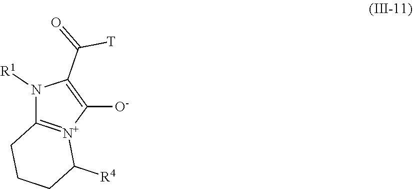

Table 12: Compounds of the formula (III-12) corresponding to the compounds of the formula II-17, in which the combination of R¹, R⁴ and T for a compound corresponds in each case to one line of Table A:

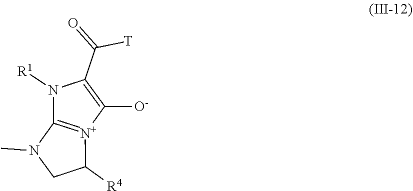

TABLE A

| No. | T | R⁴ | R¹ |
|---|---|---|---|
| A-1 | CH₃ | 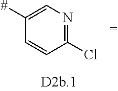 | CH₃ |
| A-2 | CH₂—Cl | D2b.1 | CH₃ |
| A-3 | CH₂—OCH₃ | D2b.1 | CH₃ |
| A-4 | CH₂—OCH(CH₃)₂ | D2b.1 | CH₃ |
| A-5 | OCH₃ | D2b.1 | CH₃ |
| A-6 | CH₂—SCH₃ | D2b.1 | CH₃ |
| A-7 | CH₂—SCH₂CH₃ | D2b.1 | CH₃ |
| A-8 | CH₂—S(O)CH₂CH₃ | D2b.1 | CH₃ |
| A-9 | CH₂—NHCH₃ | D2b.1 | CH₃ |
| A-10 | CH₂—NHCH₂CH₃ | D2b.1 | CH₃ |
| A-11 | 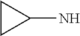 | D2b.1 | CH₃ |
| A-12 | CH₃ | 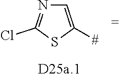 | CH₃ |
| A-13 | CH₂—Cl | D25a.1 | CH₃ |
| A-14 | CH₂—OCH₃ | D25a.1 | CH₃ |
| A-15 | CH₂—OCH(CH₃)₂ | D25a.1 | CH₃ |
| A-16 | OCH₃ | D25a.1 | CH₃ |
| A-17 | CH₂—SCH₃ | D25a.1 | CH₃ |
| A-18 | CH₂—SCH₂CH₃ | D25a.1 | CH₃ |
| A-19 | CH₂—S(O)CH₂CH₃ | D25a.1 | CH₃ |
| A-20 | CH₂—NHCH₃ | D25a.1 | CH₃ |
| A-21 | CH₂—NHCH₂CH₃ | D25a.1 | CH₃ |
| A-22 | 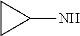 | D25a.1 | CH₃ |
| A-23 | CH₃ | 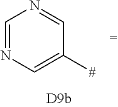 | CH₃ |
| A-24 | CH₂—Cl | D9b | CH₃ |
| A-25 | CH₂—OCH₃ | D9b | CH₃ |
| A-26 | CH₂—OCH(CH₃)₂ | D9b | CH₃ |
| A-27 | OCH₃ | D9b | CH₃ |
| A-28 | CH₂—SCH₃ | D9b | CH₃ |
| A-29 | CH₂—SCH₂CH₃ | D9b | CH₃ |
| A-30 | CH₂—S(O)CH₂CH₃ | D9b | CH₃ |
| A-31 | CH₂—NHCH₃ | D9b | CH₃ |
| A-32 | CH₂—NHCH₂CH₃ | D9b | CH₃ |
| A-33 | 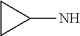 | D9b | CH₃ |
| A-34 | CH₃ | CH₂CH₂—CN | CH₃ |
| A-35 | CH₂—Cl | CH₂CH₂—CN | CH₃ |
| A-36 | CH₂—OCH₃ | CH₂CH₂—CN | CH₃ |
| A-37 | CH₂—OCH(CH₃)₂ | CH₂CH₂—CN | CH₃ |
| A-38 | OCH₃ | CH₂CH₂—CN | CH₃ |
| A-39 | CH₂—SCH₃ | CH₂CH₂—CN | CH₃ |
| A-40 | CH₂—SCH₂CH₃ | CH₂CH₂—CN | CH₃ |
| A-41 | CH₂—S(O)CH₂CH₃ | CH₂CH₂—CN | CH₃ |
| A-42 | CH₂—NHCH₃ | CH₂CH₂—CN | CH₃ |
| A-43 | CH₂—NHCH₂CH₃ | CH₂CH₂—CN | CH₃ |
| A-44 | 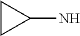 | CH₂CH₂—CN | CH₃ |
| A-45 | CH₃ | 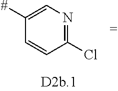 | CH₂CH₃ |
| A-46 | CH₂—Cl | D2b.1 | CH₂CH₃ |
| A-47 | CH₂—OCH₃ | D2b.1 | CH₂CH₃ |
| A-48 | CH₂—OCH(CH₃)₂ | D2b.1 | CH₂CH₃ |
| A-49 | OCH₃ | D2b.1 | CH₂CH₃ |
| A-50 | CH₂—SCH₃ | D2b.1 | CH₂CH₃ |
| A-51 | CH₂—SCH₂CH₃ | D2b.1 | CH₂CH₃ |
| A-52 | CH₂—S(O)CH₂CH₃ | D2b.1 | CH₂CH₃ |
| A-53 | CH₂—NHCH₃ | D2b.1 | CH₂CH₃ |
| A-54 | CH₂—NHCH₂CH₃ | D2b.1 | CH₂CH₃ |
| A-55 | 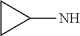 | D2b.1 | CH₂CH₃ |
| A-56 | CH₃ | 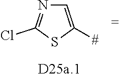 | CH₂CH₃ |
| A-57 | CH₂—Cl | D25a.1 | CH₂CH₃ |
| A-58 | CH₂—OCH₃ | D25a.1 | CH₂CH₃ |
| A-59 | CH₂—OCH(CH₃)₂ | D25a.1 | CH₂CH₃ |
| A-60 | OCH₃ | D25a.1 | CH₂CH₃ |
| A-61 | CH₂—SCH₃ | D25a.1 | CH₂CH₃ |
| A-62 | CH₂—SCH₂CH₃ | D25a.1 | CH₂CH₃ |
| A-63 | CH₂—S(O)CH₂CH₃ | D25a.1 | CH₂CH₃ |
| A-64 | CH₂—NHCH₃ | D25a.1 | CH₂CH₃ |
| A-65 | CH₂—NHCH₂CH₃ | D25a.1 | CH₂CH₃ |
| A-66 | 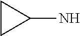 | D25a.1 | CH₂CH₃ |
| A-67 | CH₃ | 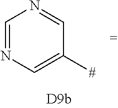 | CH₂CH₃ |
| A-68 | CH₂—Cl | D9b | CH₂CH₃ |
| A-69 | CH₂—OCH₃ | D9b | CH₂CH₃ |
| A-70 | CH₂—OCH(CH₃)₂ | D9b | CH₂CH₃ |
| A-71 | OCH₃ | D9b | CH₂CH₃ |
| A-72 | CH₂—SCH₃ | D9b | CH₂CH₃ |
| A-73 | CH₂—SCH₂CH₃ | D9b | CH₂CH₃ |
| A-74 | CH₂—S(O)CH₂CH₃ | D9b | CH₂CH₃ |
| A-75 | CH₂—NHCH₃ | D9b | CH₂CH₃ |
| A-76 | CH₂—NHCH₂CH₃ | D9b | CH₂CH₃ |
| A-77 | 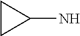 | D9b | CH₂CH₃ |
| A-78 | CH₃ | CH₂CH₂—CN | CH₂CH₃ |
| A-79 | CH₂—Cl | CH₂CH₂—CN | CH₂CH₃ |
| A-80 | CH₂—OCH₃ | CH₂CH₂—CN | CH₂CH₃ |
| A-81 | CH₂—OCH(CH₃)₂ | CH₂CH₂—CN | CH₂CH₃ |
| A-82 | OCH₃ | CH₂CH₂—CN | CH₂CH₃ |
| A-83 | CH₂—SCH₃ | CH₂CH₂—CN | CH₂CH₃ |
| A-84 | CH₂—SCH₂CH₃ | CH₂CH₂—CN | CH₂CH₃ |
| A-85 | CH₂—S(O)CH₂CH₃ | CH₂CH₂—CN | CH₂CH₃ |
| A-86 | CH₂—NHCH₃ | CH₂CH₂—CN | CH₂CH₃ |
| A-87 | CH₂—NHCH₂CH₃ | CH₂CH₂—CN | CH₂CH₃ |
| A-88 | 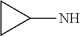 | CH₂CH₂—CN | CH₂CH₃ |
| A-89 | CH₃ | 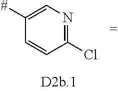 | CH(CH₃)₂ |
| A-90 | CH₂—Cl | D2b.1 | CH(CH₃)₂ |
| A-91 | CH₂—OCH₃ | D2b.1 | CH(CH₃)₂ |
| A-92 | CH₂—OCH(CH₃)₂ | D2b.1 | CH(CH₃)₂ |
| A-93 | OCH₃ | D2b.1 | CH(CH₃)₂ |
| A-94 | CH₂—SCH₃ | D2b.1 | CH(CH₃)₂ |
| A-95 | CH₂—SCH₂CH₃ | D2b.1 | CH(CH₃)₂ |
| A-96 | CH₂—S(O)CH₂CH₃ | D2b.1 | CH(CH₃)₂ |
| A-97 | CH₂—NHCH₃ | D2b.1 | CH(CH₃)₂ |
| A-98 | CH₂—NHCH₂CH₃ | D2b.1 | CH(CH₃)₂ |
| A-99 | 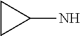 | D2b.1 | CH(CH₃)₂ |
| A-100 | CH₃ | 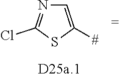 | CH(CH₃)₂ |
| A-101 | CH₂—Cl | D25a.1 | CH(CH₃)₂ |
| A-102 | CH₂—OCH₃ | D25a.1 | CH(CH₃)₂ |
| A-103 | CH₂—OCH(CH₃)₂ | D25a.1 | CH(CH₃)₂ |
| A-104 | OCH₃ | D25a.1 | CH(CH₃)₂ |
| A-105 | CH₂—SCH₃ | D25a.1 | CH(CH₃)₂ |
| A-106 | CH₂—SCH₂CH₃ | D25a.1 | CH(CH₃)₂ |
| A-107 | CH₂—S(O)CH₂CH₃ | D25a.1 | CH(CH₃)₂ |
| A-108 | CH₂—NHCH₃ | D25a.1 | CH(CH₃)₂ |
| A-109 | CH₂—NHCH₂CH₃ | D25a.1 | CH(CH₃)₂ |
| A-110 | 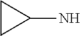 | D25a.1 | CH(CH₃)₂ |
| A-111 | CH₃ | 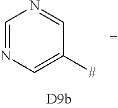 | CH(CH₃)₂ |
| A-112 | CH₂—Cl | D9b | CH(CH₃)₂ |
| A-113 | CH₂—OCH₃ | D9b | CH(CH₃)₂ |
| A-114 | CH₂—OCH(CH₃)₂ | D9b | CH(CH₃)₂ |
| A-115 | OCH₃ | D9b | CH(CH₃)₂ |
| A-116 | CH₂—SCH₃ | D9b | CH(CH₃)₂ |
| A-117 | CH₂—SCH₂CH₃ | D9b | CH(CH₃)₂ |
| A-118 | CH₂—S(O)CH₂CH₃ | D9b | CH(CH₃)₂ |
| A-119 | CH₂—NHCH₃ | D9b | CH(CH₃)₂ |
| A-120 | CH₂—NHCH₂CH₃ | D9b | CH(CH₃)₂ |
| A-121 | 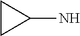 | D9b | CH(CH₃)₂ |
| A-122 | CH₃ | CH₂CH₂—CN | CH(CH₃)₂ |
| A-123 | CH₂—Cl | CH₂CH₂—CN | CH(CH₃)₂ |
| A-124 | CH₂—OCH₃ | CH₂CH₂—CN | CH(CH₃)₂ |
| A-125 | CH₂—OCH(CH₃)₂ | CH₂CH₂—CN | CH(CH₃)₂ |
| A-126 | OCH₃ | CH₂CH₂—CN | CH(CH₃)₂ |
| A-127 | CH₂—SCH₃ | CH₂CH₂—CN | CH(CH₃)₂ |
| A-128 | CH₂—SCH₂CH₃ | CH₂CH₂—CN | CH(CH₃)₂ |
| A-129 | CH₂—S(O)CH₂CH₃ | CH₂CH₂—CN | CH(CH₃)₂ |
| A-130 | CH₂—NHCH₃ | CH₂CH₂—CN | CH(CH₃)₂ |
| A-131 | CH₂—NHCH₂CH₃ | CH₂CH₂—CN | CH(CH₃)₂ |
| A-132 | 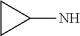 | CH₂CH₂—CN | CH(CH₃)₂ |
| A-133 | CH₃ | 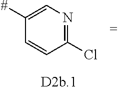 | CH₂—C₆H₅ |
| A-134 | CH₂—Cl | D2b.1 | CH₂—C₆H₅ |
| A-135 | CH₂—OCH₃ | D2b.1 | CH₂—C₆H₅ |
| A-136 | CH₂—OCH(CH₃)₂ | D2b.1 | CH₂—C₆H₅ |
| A-137 | OCH₃ | D2b.1 | CH₂—C₆H₅ |
| A-138 | CH₂—SCH₃ | D2b.1 | CH₂—C₆H₅ |
| A-139 | CH₂—SCH₂CH₃ | D2b.1 | CH₂—C₆H₅ |
| A-140 | CH₂—S(O)CH₂CH₃ | D2b.1 | CH₂—C₆H₅ |
| A-141 | CH₂—NHCH₃ | D2b.1 | CH₂—C₆H₅ |
| A-142 | CH₂—NHCH₂CH₃ | D2b.1 | CH₂—C₆H₅ |
| A-143 | 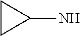 | D2b.1 | CH₂—C₆H₅ |
| A-144 | CH₃ | 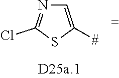 | CH₂—C₆H₅ |
| A-145 | CH₂—Cl | D25a.1 | CH₂—C₆H₅ |
| A-146 | CH₂—OCH₃ | D25a.1 | CH₂—C₆H₅ |
| A-147 | CH₂—OCH(CH₃)₂ | D25a.1 | CH₂—C₆H₅ |
| A-148 | OCH₃ | D25a.1 | CH₂—C₆H₅ |
| A-149 | CH₂—SCH₃ | D25a.1 | CH₂—C₆H₅ |
| A-150 | CH₂—SCH₂CH₃ | D25a.1 | CH₂—C₆H₅ |
| A-151 | CH₂—S(O)CH₂CH₃ | D25a.1 | CH₂—C₆H₅ |
| A-152 | CH₂—NHCH₃ | D25a.1 | CH₂—C₆H₅ |
| A-153 | CH₂—NHCH₂CH₃ | D25a.1 | CH₂—C₆H₅ |
| A-154 | 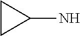 | D25a.1 | CH₂—C₆H₅ |
| A-155 | CH₃ | 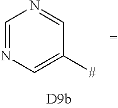 | CH₂—C₆H₅ |
| A-156 | CH₂—Cl | D9b | CH₂—C₆H₅ |
| A-157 | CH₂—OCH₃ | D9b | CH₂—C₆H₅ |
| A-158 | CH₂—OCH(CH₃)₂ | D9b | CH₂—C₆H₅ |
| A-159 | OCH₃ | D9b | CH₂—C₆H₅ |
| A-160 | CH₂—SCH₃ | D9b | CH₂—C₆H₅ |
| A-161 | CH₂—SCH₂CH₃ | D9b | CH₂—C₆H₅ |
| A-162 | CH₂—S(O)CH₂CH₃ | D9b | CH₂—C₆H₅ |
| A-163 | CH₂—NHCH₃ | D9b | CH₂—C₆H₅ |
| A-164 | CH₂—NHCH₂CH₃ | D9b | CH₂—C₆H₅ |
| A-165 | 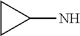 | D9b | CH₂—C₆H₅ |
| A-166 | CH₃ | CH₂CH₂—CN | CH₂—C₆H₅ |
| A-167 | CH₂—Cl | CH₂CH₂—CN | CH₂—C₆H₅ |
| A-168 | CH₂—OCH₃ | CH₂CH₂—CN | CH₂—C₆H₅ |
| A-169 | CH₂—OCH(CH₃)₂ | CH₂CH₂—CN | CH₂—C₆H₅ |
| A-170 | OCH₃ | CH₂CH₂—CN | CH₂—C₆H₅ |
| A-171 | CH₂—SCH₃ | CH₂CH₂—CN | CH₂—C₆H₅ |
| A-172 | CH₂—SCH₂CH₃ | CH₂CH₂—CN | CH₂—C₆H₅ |
| A-173 | CH₂—S(O)CH₂CH₃ | CH₂CH₂—CN | CH₂—C₆H₅ |
| A-174 | CH₂—NHCH₃ | CH₂CH₂—CN | CH₂—C₆H₅ |
| A-175 | CH₂—NHCH₂CH₃ | CH₂CH₂—CN | CH₂—C₆H₅ |
| A-176 | 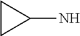 | CH₂CH₂—CN | CH₂—C₆H₅ |
| A-177 | CH₃ | 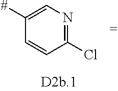 | CH₂CH═CH₂ |
| A-178 | CH₂—Cl | D2b.1 | CH₂CH═CH₂ |
| A-179 | CH₂—OCH₃ | D2b.1 | CH₂CH═CH₂ |
| A-180 | CH₂—OCH(CH₃)₂ | D2b.1 | CH₂CH═CH₂ |
| A-181 | OCH₃ | D2b.1 | CH₂CH═CH₂ |
| A-182 | CH₂—SCH₃ | D2b.1 | CH₂CH═CH₂ |
| A-183 | CH₂—SCH₂CH₃ | D2b.1 | CH₂CH═CH₂ |
| A-184 | CH₂—S(O)CH₂CH₃ | D2b.1 | CH₂CH═CH₂ |
| A-185 | CH₂—NHCH₃ | D2b.1 | CH₂CH═CH₂ |
| A-186 | CH₂—NHCH₂CH₃ | D2b.1 | CH₂CH═CH₂ |
| A-187 | 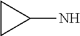 | D2b.1 | CH₂CH═CH₂ |
| A-188 | CH₃ | 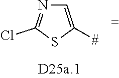 | CH₂CH═CH₂ |
| A-189 | CH₂—Cl | D25a.1 | CH₂CH═CH₂ |
| A-190 | CH₂—OCH₃ | D25a.1 | CH₂CH═CH₂ |
| A-191 | CH₂—OCH(CH₃)₂ | D25a.1 | CH₂CH═CH₂ |
| A-192 | OCH₃ | D25a.1 | CH₂CH═CH₂ |
| A-193 | CH₂—SCH₃ | D25a.1 | CH₂CH═CH₂ |
| A-194 | CH₂—SCH₂CH₃ | D25a.1 | CH₂CH═CH₂ |
| A-195 | CH₂—S(O)CH₂CH₃ | D25a.1 | CH₂CH═CH₂ |
| A-196 | CH₂—NHCH₃ | D25a.1 | CH₂CH═CH₂ |
| A-197 | CH₂—NHCH₂CH₃ | D25a.1 | CH₂CH═CH₂ |
| A-198 | 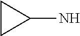 | D25a.1 | CH₂CH═CH₂ |
| A-199 | CH₃ | 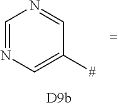 | CH₂CH═CH₂ |
| A-200 | CH₂—Cl | D9b | CH₂CH═CH₂ |
| A-201 | CH₂—OCH₃ | D9b | CH₂CH═CH₂ |
| A-202 | CH₂—OCH(CH₃)₂ | D9b | CH₂CH═CH₂ |
| A-203 | OCH₃ | D9b | CH₂CH═CH₂ |
| A-204 | CH₂—SCH₃ | D9b | CH₂CH═CH₂ |
| A-205 | CH₂—SCH₂CH₃ | D9b | CH₂CH═CH₂ |
| A-206 | CH₂—S(O)CH₂CH₃ | D9b | CH₂CH═CH₂ |
| A-207 | CH₂—NHCH₃ | D9b | CH₂CH═CH₂ |
| A-208 | CH₂—NHCH₂CH₃ | D9b | CH₂CH═CH₂ |
| A-209 | 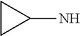 | D9b | CH₂CH═CH₂ |
| A-210 | CH₃ | CH₂CH₂—CN | CH₂CH═CH₂ |
| A-211 | CH₂—Cl | CH₂CH₂—CN | CH₂CH═CH₂ |
| A-212 | CH₂—OCH₃ | CH₂CH₂—CN | CH₂CH═CH₂ |
| A-213 | CH₂—OCH(CH₃)₂ | CH₂CH₂—CN | CH₂CH═CH₂ |
| A-214 | OCH₃ | CH₂CH₂—CN | CH₂CH═CH₂ |
| A-215 | CH₂—SCH₃ | CH₂CH₂—CN | CH₂CH═CH₂ |
| A-216 | CH₂—SCH₂CH₃ | CH₂CH₂—CN | CH₂CH═CH₂ |
| A-217 | CH₂—S(O)CH₂CH₃ | CH₂CH₂—CN | CH₂CH═CH₂ |
| A-218 | CH₂—NHCH₃ | CH₂CH₂—CN | CH₂CH═CH₂ |
| A-219 | CH₂—NHCH₂CH₃ | CH₂CH₂—CN | CH₂CH═CH₂ |
| A-220 | 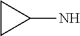 | CH₂CH₂—CN | CH₂CH═CH₂ |
| A-221 | CH₃ | 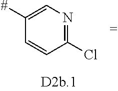 | CH₂CF₃ |
| A-222 | CH₂—Cl | D2b.1 | CH₂CF₃ |
| A-223 | CH₂—OCH₃ | D2b.1 | CH₂CF₃ |
| A-224 | CH₂—OCH(CH₃)₂ | D2b.1 | CH₂CF₃ |
| A-225 | OCH₃ | D2b.1 | CH₂CF₃ |
| A-226 | CH₂—SCH₃ | D2b.1 | CH₂CF₃ |
| A-227 | CH₂—SCH₂CH₃ | D2b.1 | CH₂CF₃ |
| A-228 | CH₂—S(O)CH₂CH₃ | D2b.1 | CH₂CF₃ |
| A-229 | CH₂—NHCH₃ | D2b.1 | CH₂CF₃ |
| A-230 | CH₂—NHCH₂CH₃ | D2b.1 | CH₂CF₃ |
| A-231 | 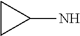 | D2b.1 | CH₂CF₃ |
| A-232 | CH₃ | 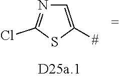 | CH₂CF₃ |
| A-233 | CH₂—Cl | D25a.1 | CH₂CF₃ |
| A-234 | CH₂—OCH₃ | D25a.1 | CH₂CF₃ |
| A-235 | CH₂—OCH(CH₃)₂ | D25a.1 | CH₂CF₃ |
| A-236 | OCH₃ | D25a.1 | CH₂CF₃ |
| A-237 | CH₂—SCH₃ | D25a.1 | CH₂CF₃ |
| A-238 | CH₂—SCH₂CH₃ | D25a.1 | CH₂CF₃ |
| A-239 | CH₂—S(O)CH₂CH₃ | D25a.1 | CH₂CF₃ |
| A-240 | CH₂—NHCH₃ | D25a.1 | CH₂CF₃ |
| A-241 | CH₂—NHCH₂CH₃ | D25a.1 | CH₂CF₃ |
| A-242 | 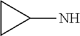 | D25a.1 | CH₂CF₃ |
| A-243 | CH₃ | 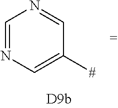 | CH₂CF₃ |
| A-244 | CH₂—Cl | D9b | CH₂CF₃ |
| A-245 | CH₂—OCH₃ | D9b | CH₂CF₃ |
| A-246 | CH₂—OCH(CH₃)₂ | D9b | CH₂CF₃ |
| A-247 | OCH₃ | D9b | CH₂CF₃ |
| A-248 | CH₂—SCH₃ | D9b | CH₂CF₃ |
| A-249 | CH₂—SCH₂CH₃ | D9b | CH₂CF₃ |
| A-250 | CH₂—S(O)CH₂CH₃ | D9b | CH₂CF₃ |
| A-251 | CH₂—NHCH₃ | D9b | CH₂CF₃ |
| A-252 | CH₂—NHCH₂CH₃ | D9b | CH₂CF₃ |
| A-253 | 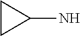 | D9b | CH₂CF₃ |
| A-254 | CH₃ | CH₂CH₂—CN | CH₂CF₃ |
| A-255 | CH₂—Cl | CH₂CH₂—CN | CH₂CF₃ |
| A-256 | CH₂—OCH₃ | CH₂CH₂—CN | CH₂CF₃ |
| A-257 | CH₂—OCH(CH₃)₂ | CH₂CH₂—CN | CH₂CF₃ |
| A-258 | OCH₃ | CH₂CH₂—CN | CH₂CF₃ |
| A-259 | CH₂—SCH₃ | CH₂CH₂—CN | CH₂CF₃ |
| A-260 | CH₂—SCH₂CH₃ | CH₂CH₂—CN | CH₂CF₃ |
| A-261 | CH₂—S(O)CH₂CH₃ | CH₂CH₂—CN | CH₂CF₃ |
| A-262 | CH₂—NHCH₃ | CH₂CH₂—CN | CH₂CF₃ |
| A-263 | CH₂—NHCH₂CH₃ | CH₂CH₂—CN | CH₂CF₃ |
| A-264 | 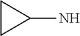 | CH₂CH₂—CN | CH₂CF₃ |

The compound of formula (I) according to the present invention can be prepared according to the following syntheses routes, e.g. according to the preparation methods and preparation schemes as described below.

The compounds of formula (I) according to the present invention can be prepared analogously to the methods described in WO2014/167084 and WO2017/093214, in particular according to the preparation methods and preparation schemes as described e.g. below.

The compounds used as starting materials for the syntheses of the compounds according to the present invention can generally be prepared by standard methods of organic chemistry. If not otherwise specified, the definitions of the variables such as R¹, R² and R³ of the structures given in the schemes have the same meaning as defined above.

Compounds of the formula (Ia) can for example be prepared by reacting the appropriately substituted compounds P-1 with halogenated ketones or acid anhydrides P-2, wherein L₁ is Br, Cl, or —O—C(═O)CH₂-hal, and hal is preferably Br or Cl, analogously to methods described in the literature (see, for example, Canadian Journal of Chemistry (1971), 49 (4), 668-671).

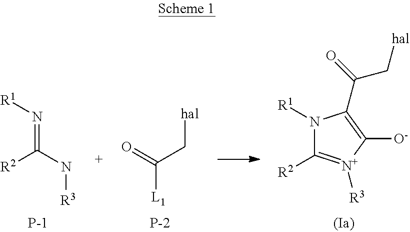

Compounds of type P-1 are known in the literature (see, for example, WO2009099929, WO2012092115, WO2011057022, WO2011017342) or can be prepared in analogy to literature known procedures. Compounds like P-2 are commercially available.

Compounds of the formula (Ib), where Alk is C₁-C₄ alkyl group, can be synthesized from P-1, by reacting with e.g. 2-chloromalonicmonoester monochloride like P-3 in the presence of base, for example, triethylamine.

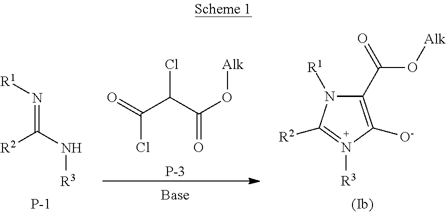

Compounds of type P-3 are known in the literature or can be prepared in analogy to literature known procedures (e.g. Tetrahedron 1993, 49, 9447-9452).

Compounds of the formula (Ic) can be obtained by reacting esters of type (Ib) with amino compounds P-4. For some amines, activation reagents like, for example, trimethylaluminium, can be applied.

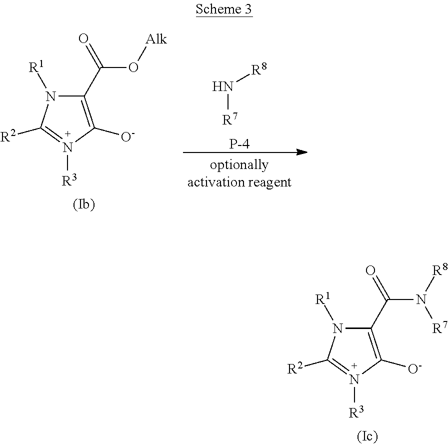

Amine compounds of type P-4 are commercially available or known in the literature.

Compounds of formula (Id) can be prepared by reacting compounds of type (Ia) with alcoholates or alcohols in the presence of base, like, for example sodium hydride, as shown in Scheme 4.

Compounds like (Id) can be transformed to compounds like (Ie) using known methods with compounds like P-5, where L₁ is Cl, Br or —O(═O)R⁵⁵ in the presence of base, like, for example, triethylamine.

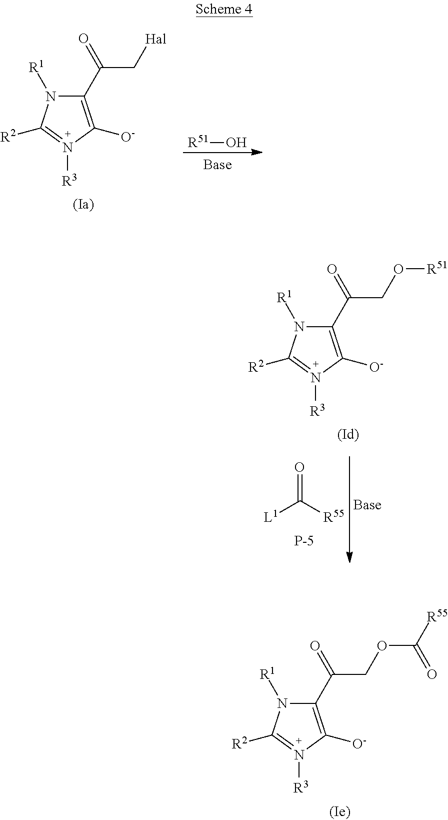

The alcohols and compounds of type P-5 are commercially available or known in the literature.

Compounds of formula (If) can be obtained from the compounds (Ia) via reaction with thiolates or thioles in the presence of base, like, for example, sodium hydride, as shown in Scheme 5. Using known methods, compounds of formula (If) can be further oxidized in the presence of oxidation agent, like, for example m-chloroperoxybenzoic acid, to sulfoxides and sulfones.

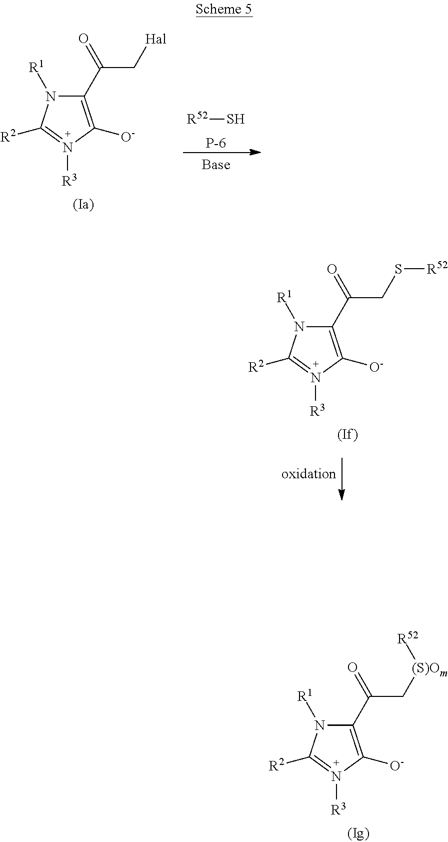

Compounds of type P-6 are commercially available or known in the literature.

Compounds of formula (Ih) can be prepared from compounds of formula (Ia) via reaction with amino compounds like P-7 using known methods in the presence of base, like, for example, triethylamine, as shown in Scheme 6. In the same manner, other amino compounds, like, for example, hydrazine could be used as well. Subsequently, compounds of formula (Ih) where L¹ is Cl, Br or —O(═O) R⁵⁵, can react with carboxylic acid halides or carboxylic acid anhydride like P-8 in the presence of base, like, for example, triethylamine, providing compounds of formula (Ii).

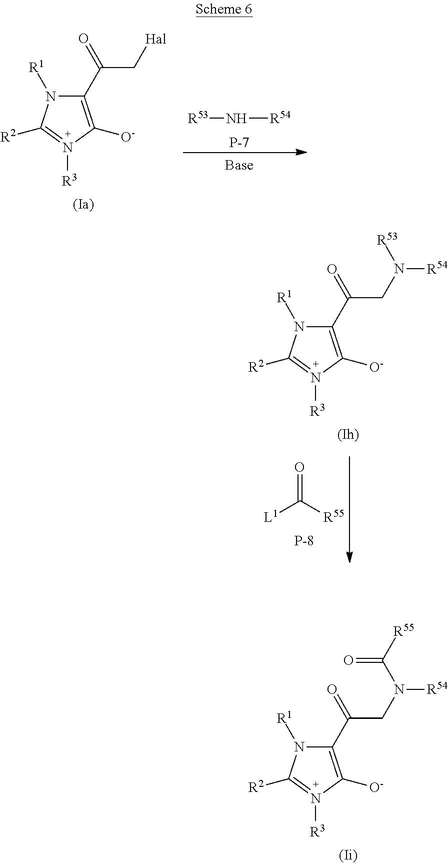

Compounds of type P-7 and P-8 are commercially available or known in the literature.

Compounds of formula (Ij) can be obtained via dehalogenation reaction of compounds of formula (Ia) using activated zinc as shown in Scheme 7 (see, for example, Dyes and Pigments 2013, 96, 7-15).

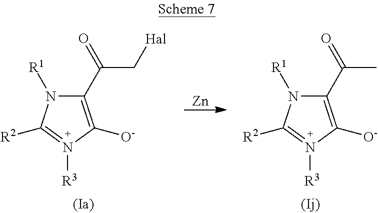

Compounds of formula (Im) can be prepared from compounds of formula (Ic) in analogy to known methods as shown in Scheme 8, using sulphurising agents, like, for example, P₂S₅ or Lawesson's reagent.

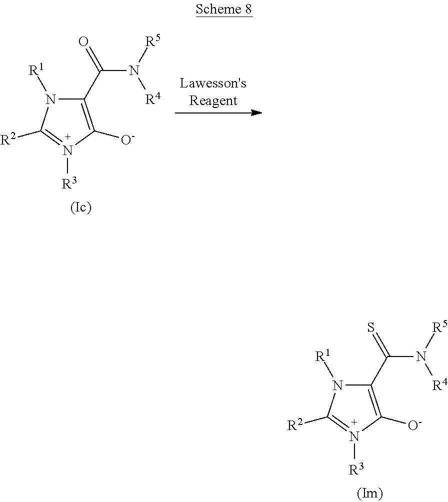

Compounds of formula (Ik) can be prepared via compounds of formula II in analogy to the reaction showed in Scheme 3. Corresponding ester compounds of formula (Ib) can react with N,O-dimethylaminohydroxylamine P-9, providing a “Weinreb-Amide” of formula (Ii). Compounds of formula (Ii) can further react with oraganometallic compounds of formula P-10, for example, alkyl- or aryllithium compounds or Grignard reagents (see, for example, WO201154844, WO2005100301) to provide desired compounds of formula (Ik) as shown in Scheme 9.

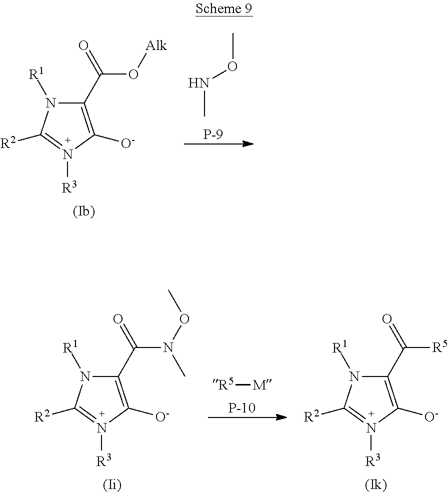

Amine compounds P-1, can also undergo reaction with carboxylic acid chlorides of type P-11 in the presence of base, in analogy to Scheme 2, providing compounds of formula (Ik) as shown in Scheme 10.

Compounds of type P-11 are either known in the literature or can be obtained from corresponding carboxylic acids (see, for example, EP 37015, DE4301356, JP2006232788).

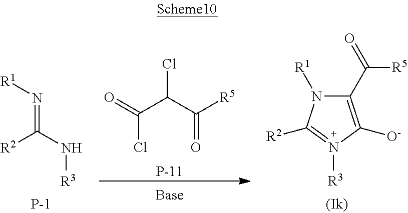

If individual compounds cannot be prepared via the above described routes, they can be prepared by derivatization of other compounds of formula (I) or by customary modifications of the synthesis routes described.

For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or silica gel.

As used herein, the term “compound(s) of the present invention” or “compound(s) according to the invention” refers to the compound(s) of formula (I) as defined above, which are also referred to as “compound(s) of formula I” or “compound(s) I” or “formula I compound(s)”, and includes their salts, tautomers, stereoisomers, and N-oxides.

Mixtures

The present invention also relates to a mixture of at least one compound of the present invention with at least one mixing partner as defined herein after. Preferred are binary mixtures of one compound of the present invention as component I with one mixing partner as defined herein after as component II. Preferred weight ratios for such binary mixtures are from 5000:1 to 1:5000, preferably from 1000:1 to 1:1000, more preferably from 100:1 to 1:100, particularly preferably from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers, and the like. Preferred mixing partners are insecticides, nematicides and fungicides.

The following list M of pesticides, grouped and numbered according the Mode of Action Classification of the Insecticide Resistance Action Committee (IRAC), together with which the compounds of the present invention can be used and with which potential synergistic effects might be produced, is intended to illustrate the possible combinations, but not to impose any limitation:

M.1 Acetylcholine esterase (AChE) inhibitors from the class of: M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of M.1 B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as: M.2A cyclodiene organochlorine compounds, as for example endosulfan or chlordane; or M.2B fiproles (phenylpyrazoles), as for example ethiprole, fipronil, flufiprole, pyrafluprole and pyriprole;

M.3 Sodium channel modulators from the class of M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin.metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or M.3B sodium channel modulators such as DDT or methoxychlor;

M.4 Nicotinic acetylcholine receptor agonists (nAChR) from the class of M.4A neonicotinoids, for example acetamiprid, clothianidin, cycloxaprid, dinotefuran, imidacloprid, nitenpyram, thiadoprid and thiamethoxam; or the compounds M.4A.2: (2E-)-1-[(6-Chloropyridin-3-yl)methyl]-N′-nitro-2-pentylidenehydrazinecarboximidamide; or M4.A.3:1-[(6-Chloropyridin-3-yl)methyl]-7-methyl-8-nitro-5-propoxy-1,2,3,5,6,7-hexahydroimidazo[1,2-a]pyridine; or from the class M.4B nicotine;

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as M.7A juvenile hormone analogues as hydroprene, kinoprene and methoprene; or others as M.7B fenoxycarb or M.7C pyriproxyfen;

M.8 miscellaneous non-specific (multi-site) inhibitors, for example M.8A alkyl halides as methyl bromide and other alkyl halides, or M.8B chloropicrin, or M.8C sulfuryl fluoride, or M.8D borax, or M.8E tartar emetic;

M.9 Selective homopteran feeding blockers, for example M.9B pymetrozine, or M.9C flonicamid;

M.10 Mite growth inhibitors, for example M.10A clofentezine, hexythiazox and diflovidazin, or

M.10B etoxazole;

M.11 Microbial disrupters of insect midgut membranes, for example Bacillus thuringiensis or Bacillus sphaericus and the insecticdal proteins they produce such as Bacillus thuringiensis subsp. israelensis, Bacillus sphaericus, Bacillus thuringiensis subsp. aizawai, Bacillus thuringiensis subsp. kurstaki and Bacillus thuringiensis subsp. tenebrionis, or the Bt crop proteins: Cry1 Ab, Cry1Ac, Cry1Fa, Cry2Ab, mCry3A, Cry3Ab, Cry3Bb and Cry34/35Ab1;

M.12 Inhibitors of mitochondrial ATP synthase, for example M.12A diafenthiuron, or M.12B organotin miticides such as azocyclotin, cyhexatin or fenbutatin oxide, or M.12C propargite, or

M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disrupters, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example M.20A hydramethylnon, or M.20B acequinocyl, or M.20C fluacrypyrim;

M.21 Mitochondrial complex I electron transport inhibitors, for example M.21A METI acaricides and insecticides such as fenazaquin, fenpyroximate, pyrimidifen, pyridaben, tebufenpyrad or tolfenpyrad, or M.21B rotenone;

M.22 Voltage-dependent sodium channel blockers, for example M.22A indoxacarb, or M.22B metaflumizone, or M.22B.1: 2-[2-(4-Cyanophenyl)-1-[3-(trifluoromethyl)phenyl]ethylidene]-N-[4-(difluoromethoxy)phenyl]-hydrazinecarboxamide or M.22B.2: N-(3-Chloro-2-methylphenyl)-2-[(4-chlorophenyl)[4-[methyl(methylsulfonyl)amino]phenyl]methylene]-hydrazinecarboxamide;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example M.24A phosphine such as aluminium phosphide, calcium phosphide, phosphine or zinc phosphide, or M.24B cyanide;

M.25 Mitochondrial complex II electron transport inhibitors, such as beta-ketonitrile derivatives, for example cyenopyrafen or cyflumetofen;

M.28 Ryanodine receptor-modulators from the class of diamides, as for example flubendiamide, chlorantraniliprole (Rynaxypyr®), Cyantraniliprole (Cyazypyr®), Tetraniliprole, or the phthalamide compounds M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.5d) and M.28.5h) to M.28.5I): M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide; M.28.5i) N-[2-(5-Amino-1,3,4-thiadiazol-2-yl)-4-chloro-6-methylphenyl]-3-bromo-1-(3-chloro-2-pyridinyl)-1H-pyrazole-5-carboxamide; M.28.5j) 3-Chloro-1-(3-chloro-2-pyridinyl)-N-[2,4-dichloro-6-[[(1-cyano-1-methylethyl)amino]carbonyl]phenyl]-1H-pyrazole-5-carboxamide; M.28.5k) 3-Bromo-N-[2,4-dichloro-6-(methylcarbamoyl)phenyl]-1-(3,5-dichloro-2-pyridyl)-1H-pyrazole-5-carboxamide; M.28.5I) N-[4-Chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; or

M.28.6: cyhalodiamide; or;

M.29. insecticidal active compounds of unknown or uncertain mode of action, as for example afidopyropen, afoxolaner, azadirachtin, amidoflumet, benzoximate, bifenazate, broflanilide, bromopropylate, chinomethionat, cryolite, dicloromezotiaz, dicofol, flufenerim, flometoquin, fluensulfone, fluhexafon, fluopyram, flupyradifurone, fluralaner, metoxadiazone, piperonyl butoxide, pyflubumide, pyridalyl, pyrifluquinazon, sulfoxaflor, tioxazafen, triflumezopyrim, or the compounds

M.29.3:11-(4-chloro-2,6-dimethylphenyl)-12-hydroxy-1,4-dioxa-9-azadispiro[4.2.4.2]-tetradec-11-en-10-one, or the compound

M.29.4: 3-(4′-fluoro-2,4-dimethylbiphenyl-3-yl)-4-hydroxy-8-oxa-1-azaspiro[4.5]dec-3-en-2-one, or the compound

M.29.5:1-[2-fluoro-4-methyl-5-[(2,2,2-trifluoroethyl)sulfinyl]phenyl]-3-(trifluoromethyl)-1H-1,2,4-triazole-5-amine, or actives on basis of Bacillus firmus (Votivo, 1-1582); or

a compound selected from the group of M.29.6, wherein the compound is selected from M.29.6a) to M.29.6k): M.29.6a) (E/Z)—N-[1-[(6-chloro-3-pyridyl)methyl]-2-pyridylidene]-2,2,2-trifluoro-acetamide; M.29.6b) (E/Z)—N-[1-[(6-chloro-5-fluoro-3-pyridyl)methyl]-2-pyridylidene]-2,2,2-trifluoro-acetamide; M.29.6c) (E/Z)-2,2,2-trifluoro-N-[1-[(6-fluoro-3-pyridyl)methyl]-2-pyridylidene]acetamide; M.29.6d) (E/Z)—N-[1-[(6-bromo-3-pyridyl)methyl]-2-pyridylidene]-2,2,2-trifluoro-acetamide; M.29.6e) (E/Z)—N-[1-[1-(6-chloro-3-pyridyl)ethyl]-2-pyridylidene]-2,2,2-trifluoro-acetamide; M.29.6f) (E/Z)—N-[1-[(6-chloro-3-pyridyl)methyl]-2-pyridylidene]-2,2-difluoro-acetamide; M.29.6g) (E/Z)-2-chloro-N-[1-[(6-chloro-3-pyridyl)methyl]-2-pyridylidene]-2,2-difluoro-acetamide; M.29.6h) (E/Z)—N-[1-[(2-chloropyrimidin-5-yl)methyl]-2-pyridylidene]-2,2,2-trifluoro-acetamide; M.29.6i) (E/Z)—N-[1-[(6-chloro-3-pyridyl)methyl]-2-pyridylidene]-2,2,3,3,3-pentafluoro-propanamide.); M.29.6j) N-[1-[(6-chloro-3-pyridyl)methyl]-2-pyridylidene]-2,2,2-trifluoro-thioacetamide; or M.29.6k) N-[1-[(6-chloro-3-pyridyl)methyl]-2-pyridylidene]-2,2,2-trifluoro-N′-isopropyl-acetamidine; or the compounds

M.29.8: fluazaindolizine; or the compounds

M.29.9.a): 4-[5-(3,5-dichlorophenyl)-5-(trifluoromethyl)-4H-isoxazol-3-yl]-2-methyl-N-(1-oxothietan-3-yl)benzamide; or M.29.9.b): fluxametamide; or

M.29.10: 5-[3-[2,6-dichloro-4-(3,3-dichloroallyloxy)phenoxy]propoxy]-1H-pyrazole; or a compound selected from the group of M.29.11, wherein the compound is selected from M.29.11b) to M.29.11p): M.29.11.b) 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide; M.29.11.c) 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide; M.29.11.d) N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.e) N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide; M.29.11.f) 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.g) 3-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.h) 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide; M.29.11.i) 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide; M.29.11.j) 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; M.29.11.k) N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; M.29.11.l) N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; M.29.11.m) N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide; M.29.11.n) 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; M.29.11.o) 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; M.29.11.p) N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; or

a compound selected from the group of M.29.12, wherein the compound is selected from M.29.12a) to M.29.12m): M.29.12.a) 2-(1,3-Dioxan-2-yl)-6-[2-(3-pyridinyl)-5-thiazolyl]-pyridine; M.29.12.b) 2-[6-[2-(5-Fluoro-3-pyridinyl)-5-thiazolyl]-2-pyridinyl]-pyrimidine; M.29.12.c) 2-[6-[2-(3-Pyridinyl)-5-thiazolyl]-2-pyridinyl]-pyrimidine; M.29.12.d) N-Methylsulfonyl-6-[2-(3-pyridyl)thiazol-5-yl]pyridine-2-carboxamide; M.29.12.e) N-Methylsulfonyl-6-[2-(3-pyridyl)thiazol-5-yl]pyridine-2-carboxamide; M.29.12.f) N-Ethyl-N-[4-methyl-2-(3-pyridyl)thiazol-5-yl]-3-methylthio-propanamide; M.29.12.g) N-Methyl-N-[4-methyl-2-(3-pyridyl)thiazol-5-yl]-3-methylthio-propanamide; M.29.12.h) N,2-Dimethyl-N-[4-methyl-2-(3-pyridyl)thiazol-5-yl]-3-methylthio-propanamide; M.29.12.i) N-Ethyl-2-methyl-N-[4-methyl-2-(3-pyridyl)thiazol-5-yl]-3-methylthio-propanamide; M.29.12.j) N-[4-Chloro-2-(3-pyridyl)thiazol-5-yl]-N-ethyl-2-methyl-3-methylthio-propanamide; M.29.12.k) N-[4-Chloro-2-(3-pyridyl)thiazol-5-yl]-N,2-dimethyl-3-methylthio-propanamide: M.29.12.l) N-[4-Chloro-2-(3-pyridyl)thiazol-5-yl]-N-methyl-3-methylthio-propanamide: M.29.12.m) N-[4-Chloro-2-(3-pyridyl)thiazol-5-yl]-N-ethyl-3-methylthio-propanamide; or the compounds

M.29.14a) 1-[(6-Chloro-3-pyridinyl)methyl]-1,2,3,5,6,7-hexahydro-5-methoxy-7-methyl-8-nitroimidazo[1,2-a]pyridine; or M.29.14b) 1-[(6-Chloropyridin-3-yl)methyl]-7-methyl-8-nitro-1,2,3,5,6,7-hexahydroimidazo[1,2-a]pyridin-5-ol; or the compounds

M.29.16a) 1-isopropyl-N,5-dimethyl-N-pyridazin-4-yl-pyrazole-4-carboxamide; or M.29.16b) 1-(1,2-dimethylpropyl)-N-ethyl-5-methyl-N-pyridazin-4-yl-pyrazole-4-carboxamide: M.29.16c) N,5-dimethyl-N-pyridazin-4-yl-1-(2,2,2-trifluoro-1-methyl-ethyl)pyrazole-4-carboxamide: M.29.16d) 1-[1-(1-cyanocyclopropyl)ethyl]-N-ethyl-5-methyl-N-pyridazin-4-yl-pyrazole-4-carboxamide; M.29.16e) N-ethyl-1-(2-fluoro-1-methyl-propyl)-5-methyl-N-pyridazin-4-yl-pyrazole-4-carboxamide; M.29.16f) 1-(1,2-dimethylpropyl)-N,5-dimethyl-N-pyridazin-4-yl-pyrazole-4-carboxamide; M.29.16g) 1-[1-(1-cyanocyclopropyl)ethyl]-N,5-dimethyl-N-pyridazin-4-yl-pyrazole-4-carboxamide; M.29.16h) N-methyl-1-(2-fluoro-1-methyl-propyl]-5-methyl-N-pyridazin-4-yl-pyrazole-4-carboxamide; M.29.16i) 1-(4,4-difluorocyclohexyl)-N-ethyl-5-methyl-N-pyridazin-4-yl-pyrazole-4-carboxamide; or M.29.16j) 1-(4,4-difluorocyclohexyl)-N,5-dimethyl-N-pyridazin-4-yl-pyrazole-4-carboxamide, or

M.29.17 a compound selected from the compounds M.29.17a) to M.29.17j): M.29.17a) N-(1-methylethyl)-2-(3-pyridinyl)-2H-indazole-4-carboxamide; M.29.17b) N-cyclopropyl-2-(3-pyridinyl)-2H-indazole-4-carboxamide; M.29.17c) N-cyclohexyl-2-(3-pyridinyl)-2H-indazole-4-carboxamide; M.29.17d) 2-(3-pyridinyl)-N-(2,2,2-trifluoroethyl)-2H-indazole-4-carboxamide; M.29.17e) 2-(3-pyridinyl)-N-[(tetrahydro-2-furanyl)methyl]-2H-indazole-5-carboxamide; M.29.17f) methyl 2-[[2-(3-pyridinyl)-2H-indazol-5-yl]carbonyl]hydrazinecarboxylate; M.29.17g) N-[(2,2-difluorocyclopropyl)methyl]-2-(3-pyridinyl)-2H-indazole-5-carboxamide; M.29.17h) N-(2,2-difluoropropyl)-2-(3-pyridinyl)-2H-indazole-5-carboxamide; M.29.17i) 2-(3-pyridinyl)-N-(2-pyrimidinylmethyl)-2H-indazole-5-carboxamide; M.29.17j) N-[(5-methyl-2-pyrazinyl)methyl]-2-(3-pyridinyl)-2H-indazole-5-carboxamide, or M.29.18 a compound selected from the compounds M.29.18a) to M.29.18d): M.29.18a) N-[3-chloro-1-(3-pyridyl)pyrazol-4-yl]-N-ethyl-3-(3,3,3-trifluoropropylsulfanyl)propanamide; M.29.18b) N-[3-chloro-1-(3-pyridyl)pyrazol-4-yl]-N-ethyl-3-(3,3,3-trifluoropropylsulfinyl)propanamide; M.29.18c) N-[3-chloro-1-(3-pyridyl)pyrazol-4-yl]-3-[(2,2-difluorocyclopropyl)methylsulfanyl]-N-ethyl-propanamide; M.29.18d) N-[3-chloro-1-(3-pyridyl)pyrazol-4-yl]-3-[(2,2-difluorocyclopropyl)methylsulfinyl]-N-ethyl-propanamide; or the compound

M.29.19 sarolaner, or the compound

M.29.20 lotilaner.

The commercially available compounds of the group M listed above may be found in The Pesticide Manual, 16th Edition, C. MacBean, British Crop Protection Council (2013) among other publications. The online Pesticide Manual is updated regularly and is accessible through http://bcpcdata.com/pesticide-manual.html.

Another online data base for pesticides providing the ISO common names is http://www.alanwood.net/pesticides.

The M.4 neonicotinoid cycloxaprid is known from WO2010/069266 and WO2011/069456, the neonicotinoid M.4A.2, sometimes also to be named as guadipyr, is known from WO2013/003977, and the neonicotinoid M.4A.3 (approved as paichongding in China) is known from WO2007/101369. The metaflumizone analogue M.22B.1 is described in CN10171577 and the analogue M.22B.2 in CN102126994. The phthalimides M.28.1 and M.28.2 are both known from WO2007/101540. The anthranilamide M.28.3 is described in WO2005/077934. The hydrazide compound M.28.4 is described in WO2007/043677. The anthranilamides M.28.5a) to M.28.5d) and M.28.5h) are described in WO 2007/006670, WO2013/024009 and WO2013/024010, the anthranilamide M.28.5i) is described in WO2011/085575, M.28.5j) in WO2008/134969, M.28.5k) in US2011/046186 and M.28.5I) in WO2012/034403. The diamide compound M.28.6 can be found in WO2012/034472. The spiroketal-substituted cyclic ketoenol derivative M.29.3 is known from WO2006/089633 and the biphenyl-substituted spirocyclic ketoenol derivative M.29.4 from WO2008/067911. The triazoylphenylsulfide M.29.5 is described in WO2006/043635, and biological control agents on the basis of Bacillus firmus are described in WO2009/124707. The compounds M.29.6a) to M.29.6i) listed under M.29.6 are described in WO2012/029672, and M.29.6j) and M.29.6k) in WO2013/129688. The nematicide M.29.8 is known from WO2013/055584. The isoxazoline M.29.9.a) is described in WO2013/050317. The isoxazoline M.29.9.b) is described in WO2014/126208. The pyridalyl-type analogue M.29.10 is known from WO2010/060379. The carboxamides broflanilide and M.29.11.b) to M.29.11.h) are described in WO2010/018714, and the carboxamides M.29.11 i) to M.29.11.p) in WO2010/127926. The pyridylthiazoles M.29.12.a) to M.29.12.C) are known from WO2010/006713, M.29.12.d) and M.29.12.e) are known from WO2012/000896, and M.29.12.f) to M.29.12.m) from WO2010/129497. The compounds M.29.14a) and M.29.14b) are known from WO2007/101369. The pyrazoles M.29.16.a) to M.29.16h) are described in WO2010/034737, WO2012/084670, and WO2012/143317, respectively, and the pyrazoles M.29.16i) and M.29.16j) are described in U.S. 61/891,437. The pyridinylindazoles M.29.17a) to M.29.17.j) are described in WO2015/038503. The pyridylpyrazoles M.29.18a) to M.29.18d) are described in US2014/0213448. The isoxazoline M.29.19 is described in WO2014/036056. The isoxazoline M.29.20 is known from WO2014/090918.

The following list of fungicides, in conjunction with which the compounds of the present invention can be used, is intended to illustrate the possible combinations but does not limit them:

A) Respiration Inhibitors
Inhibitors of complex III at Qₒ site (e. g. strobilurins): azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxy.strobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), tridopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A.1.23), 1-[3-bromo-2-[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27), 1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyl-tetrazol-5-one (A.1.29), 1-[3-cyclopropyl-2-[[2-methyl-4-(1-methylpyrazol-3-yl)phenoxy]methyl]phenyl]-4-methyl-tetrazol-5-one (A.1.30), 1-[3-(difluoromethoxy)-2-[[2-methyl-4-(1-methylpyrazol-3-yl)phenoxy]methyl]phenyl]-4-methyl-tetrazol-5-one (A.1.31), 1-methyl-4-[3-methyl-2-[[2-methyl-4-(1-methylpyrazol-3-yl)phenoxy]methyl]phenyl]tetrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), (Z,2E)-5-[1-(4-chloro-2-fluoro-phenyl)pyrazol-3-yl]oxy-2-methoxyimino-N,3-dimethyl-pent-3-enamide (A.1.36),
inhibitors of complex III at Qi site: cyazofamid (A.2.1), amisulbrom (A.2.2), [(3S,6S,7R,8R)-8-benzyl-3-[(3-acetoxy-4-methoxy-pyridine-2-carbonyl)amino]-6-methyl-4,9-dioxo-1,5-dioxonan-7-yl] 2-methylpropanoate (A.2.3), [(3S,6S,7R,8R)-8-benzyl-3-[[3-(acetoxymethoxy)-4-methoxypyridine-2-carbonyl]amino]-6-methyl-4,9-dioxo-1,5-dioxonan-7-yl] 2-methylpropanoate (A.2.4), [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate (A.2.5), [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate (A.2.7), (3S,6S,7R,8R)-8-benzyl-3-[3-[(isobutyryloxy)methoxy]-4-methoxypicolinamido]-6-methyl-4,9-dioxo-1,5-dioxonan-7-yl isobutyrate (A.2.8);
inhibitors of complex II (e. g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoromethylthiobiphenyl-2-yl)-3-difluoromethyl-1-methyl-1H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoromethyl)-1-methyl-N-(1,1,3-trimethylindan-4-yl)pyrazole-4-carboxamide (A.3.22), 1,3-dimethyl-N-(1,1,3-trimethylindan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);
other respiration inhibitors (e. g. complex I, uncouplers): diflumetorim (A.4.1), (5,8-difluoroquinazolin-4-yl)-{2-[2-fluoro-4-(4-trifluoromethylpyridin-2-yloxy)-phenyl]-ethyl}-amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

B) Sterol Biosynthesis Inhibitors (SBI Fungicides)
C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S;3R)-3-(2-chlorophenyl)-2-(2,4-difluorophenyl)-oxiranylmethyl]-5-thiocyanato-1H-[1,2,4]triazolo (B.1.31), 2-[rel-(2S;3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chlorophenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.51); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol-4-yl]-(3-pyridyl)methanol (B.1.50);
Delta 14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);

C) Nucleic Acid Synthesis Inhibitors
phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);
others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

D) Inhibitors of Cell Division and Cytoskeleton
tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);
other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

E) Inhibitors of Amino Acid and Protein Synthesis
methionine synthesis inhibitors (anilino-pyrimidines): cyprodinil (E.1.1), mepanipyrim (E.1.2), pyrimethanil (E.1.3);
protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

F) Signal Transduction Inhibitors
MAP/histidine kinase inhibitors: fluoroimid (F.1.1), iprodione (F.1.2), procymidone (F.1.3), vinclozolin (F.1.4), fenpiclonil (F.1.5), fludioxonil (F.1.6);
G protein inhibitors: quinoxyfen (F.2.1);

G) Lipid and Membrane Synthesis Inhibitors
Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenyl)ethanesulfonyl)-but-2-yl) carbamic acid-(4-fluorophenyl) ester (G.3.8);
compounds affecting cell membrane permeability and fatty acides: propamocarb (G.4.1);
fatty acid amide hydrolase inhibitors: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3);

H) Inhibitors with Multi Site Action
inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds (e. g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenoie (H.3.9) and its salts, phthalide (H.3.10), tolytfluanid (H.3.11), N-(4-chloro-2-nitro-phenyl)-N-ethyl-4-methyl-benzenesulfonamide (H.3.12);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

I) Cell Wall Synthesis Inhibitors
inhibitors of glucan synthesis: validamycin (1.1.1), polyoxin B (1.1.2);
melanin synthesis inhibitors: pyroquilon (1.2.1), tricyclazole (I.2.2), carpropamid (I.2.3), dicyclomet (I.2.4), fenoxanil (I.2.5);

J) Plant Defence Inducers
acibenzolar-S-methyl (J.1.1), probenazoie (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9);

K) Unknown Mode of Action
bronopoi (K.1.1), chinomethionat (K.1.2), cyflufenamid (K.1.3), cymoxanil (K.1.4), dazomet (K.1.5), debacarb (K.1.6), diclomezine (K.1.7), difenzoquat (K.1.8), difenzoquat-methylsulfate (K.1.9), diphenylamin (K.1.10), fenpyrazamine (K.1.11), flumetover (K.1.12), flusuifamide (K.1.13), flutianil (K.1.14), methasulfocarb (K.1.15), nitrapyrin (K.1.16), nitrothal-isopropyl (K.1.18), oxathiapiprolin (K.1.19), toiprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K.1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K.1.27), 2-[3,5-bis(difluoromethyl)-1H-pyrazol-1-yl]-1-[4-(4-{5-[2-fluoro-6-(prop-2-yn-1-yl-oxy)phenyl]-4,5-dihydro-1,2-oxazol-3-yl}-1,3-thiazol-2-yl)piperidin-1-yl]ethanone (K.1.28), 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone (K.1.29), N-(cyclopropylmethoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-trimethylsilanyl-propoxy)-phenyl)-N-ethyl-N-methyl formamidine (K.1.33), N′-(5-difluoromethyl-2-methyl-4-(3-trimethylsilanyl-propoxy)-phenyl)-N-ethyl-N-methyl formamidine (K.1.34), methoxy-acetic acid 6-tert-butyl-8-fluoro-2,3-dimethylquinolin-4-yl ester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dimethoxy-pyrimidin-2-yl)-2-methyl-1H-benzoimidazole (K.1.39), 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluoro-phenyl]propan-2-oi (K.1.43), 2-[2-fluoro-6-[(8-fluoro-2-methyl-3-quinolyl)oxy]phen-yl]propan-2-ol (K.1.44), 3-(5-fluoro-3,3,4,4-tetramethyl-3,4-dihydroisoquinolin-1-yl)quinoline (K.1.45), 3-(4,4-difluoro-3,3-dimethyl-3,4-dihydroisoquinolin-1-yl)quinoline (K.1.46), 3-(4,4,5-trifluoro-3,3-dimethyl-3,4-dihydroisoquinolin-1-yl)quinoline (K.1.47), 9-fluoro-2,2-dimethyl-5-(3-quinolyl)-3H-1,4-benzoxazepine (K.1.48).

The fungicides described by common names, their preparation and their activity e.g. against harmful fungi is known (cf.: http://www.alanwood.net/pesticides/); these substances are commercially available.

The fungicides described by IUPAC nomenclature, their preparation and their pesticidal activity is also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, WO 13/127704, WO 13/024009, WO 13/024010 and WO 13/047441, WO 13/162072, WO 13/092224, WO 11/135833).

Biopesticides

Suitable mixing partners for the compounds of the present invention also include biopesticides.

Biopesticides have been defined as a form of pesticides based on micro-organisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:

(1) Microbial pesticides consist of bacteria, fungi or viruses (and often include the metabolites that bacteria and fungi produce). Entomopathogenic nematodes are also classified as microbial pesticides, even though they are multi-cellular.

(2) Biochemical pesticides are naturally occurring substances or structurally-similar and functionally identical to a naturally-occurring substance and extracts from biological sources that control pests or provide other crop protection uses as defined below, but have non-toxic mode of actions (such as growth or developmental regulation, attractents, repellents or defence activators (e.g. induced resistance) and are relatively non-toxic to mammals.

Biopesticides for use against crop diseases have already established themselves on a variety of crops. For example, biopesticides already play an important role in controlling downy mildew diseases. Their benefits include: a 0-Day Pre-Harvest Interval, the ability to use under moderate to severe disease pressure, and the ability to use in mixture or in a rotational program with other registered pesticides.

A major growth area for biopesticides is in the area of seed treatments and soil amendments. Biopesticidal seed treatments are e.g. used to control soil borne fungal pathogens that cause seed rots, damping-off, root rot and seedling blights. They can also be used to control internal seed borne fungal pathogens as well as fungal pathogens that are on the surface of the seed. Many biopesticidal products also show capacities to stimulate plant host defenses and other physiological processes that can make treated crops more resistant to a variety of biotic and abiotic stresses or can regulate plant growth. Many biopesticidal products also show capacities to stimulate plant health, plant growth and/or yield enhancing activity.

The following list of biopesticides, in conjunction with which the compounds of the present invention can be used, is intended to illustrate the possible combinations but does not limit them:

L) Biopesticides

L1) Microbial pesticides with fungicidal, bactericidal, viricidal and/or plant defense activator activity: Ampelomyces quisqualis, Aspergillus flavus, Aureobasidium pullulans, Bacillus altitudinis, B. amyloliquefaciens, B. megaterium, B. mojavensis, B. mycoides, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus alvei, Paenibacillus polymyxa, Pantoea vagans, Penicillium bilaiae, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperelloides, T. asperellum, T. atroviride, T. fertile, T. gamsii, T. harmatum, T. harzianum, T. polysporum, T. stromaticum, T. virens, T. viride, Typhula phacorrhiza, Ulocladium oudemansii, Verticillium dahlia, zucchini yellow mosaic virus (avirulent strain);

L2) Biochemical pesticides with fungicidal, bactericidal, viricidal and/or plant defense activator activity: harpin protein, Reynoutria sachalinensis extract;

L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartll, Burkholderia spp., Chromobacterium subtsugae, Cydia pornonails granulovirus (CpGV), Cryptophlebia leucotreta granulovirus (CrleGV), Flavobacterium spp., Helicoverpa armigera nucleopolyhedrovirus (HearNPV), Helicoverpa zea nucleopolyhedrovirus (HzNPV), Helicoverpa zea single capsid nucleopolyhedrovirus (HzSNPV), Heterorhabditis bacteriophora, Isaris fumosorosea, Lecanicillium longisporum, L. muscarium, Metarhizium anisopliae, Metarhizium anisopliae var. anisopliae, M anisopliae var. acridum, Nomuraea rileyi, Paecilomyces fumosoroseus, P. lilacinus, Paenibacillus popilliae, Pasteuria spp., P. nishizawae, P. penetrans, P. ramosa, P. thornea, P. usgae, Pseudomonas fluorescens, Spodoptera littoralis nucleopolyhedrovirus (SpliNPV), Steinernema carpocapsae, S. feltiae, S. kraussei, Streptomyces galbus, S. microflavus;

L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, extract of Chenopodium ambrosiodes, Neem oil, Quillay extract;

L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium spp., B. elkanii, B. japonicum, B. liaoningense, B. lupini, Delftia acidovorans, Glomus intraradices, Mesorhizobium spp., Rhizobium leguminosarum bv. phaseoli, R. I bv. trifolii, R. Z bv. viciae, R. tropici, Sinorhizobium meliloti.

The biopesticides from group L1) and/or L2) may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group L5) may also have fungicidal, bactericidal, viricidal, plant defense activator, insecticidal, acaricidal, molluscidal, pheromone and/or nematicidal activity.

Many of these biopesticides have been deposited under deposition numbers mentioned herein (the prefices such as ATCC or DSM refer to the acronym of the respective culture collection, for details see e. g. here: http://www.wfcc.info/ccinfo/collection/by_acronym/), are referred to in literature, registered and/or are commercially available: mixtures of Aureobasidium pullulans DSM 14940 and DSM 14941 isolated in 1989 in Konstanz, Germany (e. g. blastospores in BlossomProtect® from bio-ferm GmbH, Austria), Azospirillum brasilense Sp245 originally isolated in wheat reagion of South Brazil (Passo Fundo) at least prior to 1980 (BR 11005; e. g. GELFIX® Gramineas from BASF Agricultural Specialties Ltd., Brazil), A. brasilense strains Ab-V5 and Ab-V6 (e. g. in AzoMax from Novozymes BioAg Produtos papra Agricultura Ltda., Quattro Barras, Brazil or Simbiose-Maiz® from Simbiose-Agro, Brazil; Plant Soil 331, 413-425, 2010), Bacillus amyloliquefaciens strain AP-188 (NRRL B-50615 and B-50331; U.S. Pat. No. 8,445,255); B. amyloliquefaciens spp. plantarum D747 isolated from air in Kikugawa-shi, Japan (US 20130236522 A1; FERM BP-8234; e. g. Double Nickell™ 55 WDG from Certis LLC, USA), B. amyloliquefaciens spp. plantarum FZB24 isolated from soil in Brandenburg, Germany (also called SB3615; DSM 96-2; J. Plant Dis. Prot. 105, 181-197, 1998; e. g. Taegro® from Novozyme Biologicals, Inc., USA), B. amyloliquefaciens ssp. plantarum FZB42 isolated from soil in Brandenburg, Germany (DSM 23117; J. Plant Dis. Prot. 105, 181-197, 1998; e. g. RhizoVital® 42 from AbiTEP GmbH, Germany), B. amyloliquefaciens ssp. plantarum MBI600 isolated from faba bean in Sutton Bonington, Nottinghamshire, U.K. at least before 1988 (also called 1430; NRRL B-50595; US 2012/0149571 A1; e. g. Integral® from BASF Corp., USA), B. amyloliquefaciens spp. plantarum QST-713 isolated from peach orchard in 1995 in California, U.S.A. (NRRL B-21661; e. g. Serenade® MAX from Bayer Crop Science LP, USA), B. amyloliquefaciens spp. plantarum TJ1000 isolated in 1992 in South Dakoda, U.S.A. (also called 1BE; ATCC BAA-390; CA 2471555 A1; e. g. QuickRoots™ from TJ Technologies, Watertown, S. Dak., USA), B. firmus CNCM 1-1582, a variant of parental strain EIP-N1 (CNCM 1-1556) isolated from soil of central plain area of Israel (WO 2009/126473, U.S. Pat. No. 6,406,690; e. g. Votivo® from Bayer CropScience LP, USA), B. pumllus GHA 180 isolated from apple tree rhizosphere in Mexico (IDAC 260707-01; e. g. PRO-MIX® BX from Premier Horticulture, Quebec, Canada), B. pumllus INR-7 otherwise referred to as BU-F22 and BU-F33 isolated at least before 1993 from cucumber infested by Erwinia tracheiphila (NRRL B-50185, NRRL B-50153; U.S. Pat. No. 8,445,255), B. pumllus KFP9F isolated from the rhizosphere of grasses in South Africa at least before 2008 (NRRL B-50754; WO 2014/029697; e. g. BAC-UP or FUSION-P from BASF Agricultural Specialities (Pty) Ltd., South Africa), B. pumllus Q ST 2808 was isolated from soil collected in Pohnpei, Federated States of Micronesia, in 1998 (NRRL B-30087; e. g. Sonata® or Ballad® Plus from Bayer Crop Science LP, USA), B. simplex ABU 288 (NRRL B-50304; U.S. Pat. No. 8,445,255), B. subtilis FB17 also called UD 1022 or UD10-22 isolated from red beet roots in North America (ATCC PTA-11857; System. Appl. Microbiol. 27, 372-379, 2004; US 2010/0260735; WO 2011/109395); B. thuringiensis ssp. aizawai ABTS-1857 isolated from soil taken from a lawn in Ephraim, Wis., U.S.A., in 1987 (also called ABG-6346; ATCC SD-1372; e. g. XenTari® from BioFa AG, Munsingen, Germany), B. t. ssp. kurstaki ABTS-351 identical to HD-1 isolated in 1967 from diseased Pink Bollworm black larvae in Brownsville, Tex., U.S.A. (ATCC SD-1275; e. g. Dipel® DF from Valent BioSciences, IL, USA), B. t. ssp. kurstaki SB4 isolated from E. saccharina larval cadavers (NRRL B-50753; e. g. Beta Pro® from BASF Agricultural Specialities (Pty) Ltd., South Africa), B. t. ssp. tenebrionis NB-176-1, a mutant of strain NB-125, a wild type strain isolated in 1982 from a dead pupa of the beetle Tenebrio molitor (DSM 5480; EP 585 215 B1; e. g. Novodor® from Valent BioSciences, Switzerland), Beauveria bassiana GHA (ATCC 74250; e. g. BotaniGard® 22WGP from Laverlam Int. Corp., USA), B. bassiana JW-1 (ATCC 74040; e. g. Naturalis® from CBC (Europe) S.r.I., Italy), B. bassiana PPRI 5339 isolated from the larva of the tortoise beetle Conchyloctenia punctata (NRRL 50757; e. g. BroadBand® from BASF Agricultural Specialities (Pty) Ltd., South Africa), Bradyrhizobium elkanii strains SEMIA 5019 (also called 29W) isolated in Rio de Janeiro, Brazil and SEMIA 587 isolated in 1967 in the State of Rio Grande do Sul, from an area previously inoculated with a North American isolate, and used in commercial inoculants since 1968 (Appl. Environ. Microbiol. 73(8), 2635, 2007; e. g. GELFIX 5 from BASF Agricultural Specialties Ltd., Brazil), B. japonicum 532c isolated from Wisconsin field in U.S.A. (Nitragin 61A152; Can. J. Plant. Sci. 70, 661-666, 1990; e. g. in Rhizoflo®, Histick®, Hicoat® Super from BASF Agricultural Specialties Ltd., Canada), B. japonicum E-109 variant of strain USDA 138 (INTA E109, SEMIA 5085; Eur. J. Soil Biol. 45, 28-35, 2009; Biol. Fertil. Soils 47, 81-89, 2011); B. japonicum strains deposited at SEMIA known from Appl. Environ. Microbiol. 73(8), 2635, 2007: SEMIA 5079 isolated from soil in Cerrados region, Brazil by Embrapa-Cerrados used in commercial inoculants since 1992 (CPAC 15; e. g. GELFIX 5 or ADHERE 60 from BASF Agricultural Specialties Ltd., Brazil), B. japonicum SEMIA 5080 obtained under lab conditions by Embrapa-Cerrados in Brazil and used in commercial inoculants since 1992, being a natural variant of SEMIA 586 (CB1809) originally isolated in U.S.A. (CPAC 7; e. g. GELFIX 5 or ADHERE 60 from BASF Agricultural Specialties Ltd., Brazil); Burkholderia sp. A396 isolated from soil in Nikko, Japan, in 2008 (NRRL B-50319; WO 2013/032693; Marrone Bio Innovations, Inc., USA), Coniothyrium minitans CON/M/91-08 isolated from oilseed rape (WO 1996/021358; DSM 9660; e. g. Contans® WG, Intercept® WG from Bayer CropScience AG, Germany), harpin (alpha-beta) protein (Science 257, 85-88, 1992; e. g. Messenger™ or HARP-N-Tek from Plant Health Care plc, U.K.), Helicoverpa armigera nucleopolyhedrovirus (HearNPV) (J. Invertebrate Pathol. 107, 112-126, 2011; e. g. Helicovex® from Adermatt Biocontrol, Switzerland; Diplomata® from Koppert, Brazil; Vivus® Max from AgBiTech Pty Ltd., Queensland, Australia), Helicoverpa zea single capsid nucleopolyhedrovirus (HzSNPV) (e. g. Gemstar® from Certis LLC, USA), Helicoverpa zea nucleopolyhedrovirus ABA-NPV-U (e. g. Heligen® from AgBiTech Pty Ltd., Queensland, Australia), Heterorhabditis bacteriophora (e. g. Nemasys® G from BASF Agricultural Specialities Limited, UK), tsar/a fumosorosea Apopka-97 isolated from mealy bug on gynura in Apopka, Fla., U.S.A. (ATCC 20874; Biocontrol Science Technol. 22(7), 747-761, 2012; e. g. PFR-97™ or PreFeRal® from Certis LLC, USA), Metarhizium anisopliae var. anisopliae F52 also called 275 or V275 isolated from codling moth in Austria (DSM 3884, ATCC 90448; e. g. Met52® Novozymes Biologicals BioAg Group, Canada), Metschnikowia fructicola 277 isolated from grapes in the central part of Israel (U.S. Pat. No. 6,994,849; NRRL Y-30752; e. g. formerly Shemer® from Agrogreen, Israel), Paecilomyces ilacinus 251 isolated from infected nematode eggs in the Philippines (AGAL 89/030550; WO1991/02051; Crop Protection 27, 352-361, 2008; e. g. BioAct® from Bayer CropScience AG, Germany and MeloCon® from Certis, USA), Paenibacillus alvei NAS6G6 isolated from the rhizosphere of grasses in South Africa at least before 2008 (WO 2014/029697; NRRL B-50755; e.g. BAC-UP from BASF Agricultural Specialities (Pty) Ltd., South Africa), Pasteur/a nishizawae Pn1 isolated from a soybean field in the mid-2000s in Illinois, U.S.A. (ATCC SD-5833; Federal Register 76(22), 5808, Feb. 2, 2011; e.g. Clariva™ PN from Syngenta Crop Protection, LLC, USA), Penicillium bilaiae (also called P. bilaii) strains ATCC 18309 (=ATCC 74319), ATCC 20851 and/or ATCC 22348 (=ATCC 74318) originally isolated from soil in Alberta, Canada (Fertilizer Res. 39, 97-103, 1994; Can. J. Plant Sci. 78(1), 91-102, 1998; U.S. Pat. No. 5,026,417, WO 1995/017806; e. g. Jump Start®, Provide® from Novozymes Biologicals BioAg Group, Canada), Reynoutria sachalinensis extract (EP 0307510 B1; e. g. Regalia® SC from Marrone BioInnovations, Davis, Calif., USA or Milsana® from BioFa AG, Germany), Steinernema carpocapsae (e. g. Millenium® from BASF Agricultural Specialities Limited, UK), S. feltiae (e. g. Nemashield® from BioWorks, Inc., USA; Nemasys® from BASF Agricultural Specialities Limited, UK), Streptomyces microflavus NRRL B-50550 (WO 2014/124369; Bayer CropScience, Germany), Trichoderma asperelloides JM41R isolated in South Africa (NRRL 50759; also referred to as T. fertile; e. g. Trichoplus® from BASF Agricultural Specialities (Pty) Ltd., South Africa), T. harzianum T-22 also called KRL-AG2 (ATCC 20847; BioControl 57, 687-696, 2012; e. g. Plantshield® from BioWorks Inc., USA or SabrEx™ from Advanced Biological Marketing Inc., Van Wert, Ohio, USA).

According to the invention, the solid material (dry matter) of the biopesticides (with the exception of oils such as Neem oil) are considered as active components (e.g. to be obtained after drying or evaporation of the extraction or suspension medium in case of liquid formulations of the microbial pesticides).

In accordance with the present invention, the weight ratios and percentages used herein for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10¹⁰ CFU equals one gram of total weight of the respective active component. Colony forming unit is measure of viable microbial cells, in particular fungal and bacterial cells. In addition, here “CFU” may also be understood as the number of (juvenile) individual nematodes in case of (entomopathogenic) nematode biopesticides, such as Steinernema feltiae.

When mixtures comprising microbial pesticides are employed in crop protection, the application rates preferably range from about 1×106 to 5×1015 (or more) CFU/ha, preferably from about 1×108 to about 1×1013 CFU/ha, and even more preferably from about 1×109 to about 1×1012 CFU/ha. In the case of (entomopathogenic) nematodes as microbial pesticides (e. g. Steinernema feltiae), the application rates preferably range inform about 1×105 to 1×1012 (or more), more preferably from 1×108 to 1×1011, even more preferably from 5×108 to 1×1010 individuals (e. g. in the form of eggs, juvenile or any other live stages, preferably in an infective juvenile stage) per ha.

When mixtures comprising microbial pesticides are employed in seed treatment, the application rates with respect to plant propagation material preferably range from about 1×106 to 1×1012 (or more) CFU/seed. Preferably, the concentration is about 1×106 to about 1×109 CFU/seed. In the case of the microbial pesticides II, the application rates with respect to plant propagation material also preferably range from about 1×107 to 1×1014 (or more) CFU per 100 kg of seed, preferably from 1×109 to about 1×1012 CFU per 100 kg of seed.

Formulations

The invention also relates to agrochemical compositions comprising an auxiliary and at least one compound of the present invention or a mixture thereof.

An agrochemical composition comprises a pesticidally effective amount of a compound of the present invention or a mixture thereof. The term “pesticidally effective amount” is defined below.

The compounds of the present invention or the mixtures thereof can be converted into customary types of agro-chemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclo¬hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.)

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Exam-pies of carboxylates are alkyl carboxylates, and carboxylated alcohol or alkylphenol eth-oxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

ii) Dispersible Concentrates (DC)

5-25 wt % of a compound I according to the invention and 1-10 wt % dispersant (e. g. polyvinylpyrrolidone) are dissolved in up to 100 wt % organic solvent (e.g. cyclohexanone). Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

vi) Water-Dispersible Granules and Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

ix) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

x) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radi-cal initiator results in the formation of poly(meth)acrylate microcapsules. Alternatively, an oil phase comprising 5-50 wt % of a compound I according to the invention, 0-40 wt % water insolu-ble organic solvent (e.g. aromatic hydrocarbon), and an isocyanate monomer (e.g. diphenylme-thene-4,4′-diisocyanatae) are dispersed into an aqueous solution of a protective colloid (e.g. polyvinyl alcohol). The addition of a polyamine (e.g. hexamethylenediamine) results in the for-mation of a polyurea microcapsule. The monomers amount to 1-10 wt %. The wt % relate to the total CS composition.

xi) Dustable Powders (DP, DS)

1-10 wt % of a compound I according to the invention are ground finely and mixed intimately with up to 100 wt % solid carrier, e.g. finely divided kaolin.

xii) Granules (GR, FG)

0.5-30 wt % of a compound I according to the invention is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I according to the invention are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and most preferably between 0.5 and 75%, by weight of active sub-stance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage de-vice, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds of the present invention and/or mixing partners as defined above, may be mixed by the user in a spray tank and further auxiliaries and additives may be added, if appropriate.

In a further embodiment, either individual components of the composition according to the invention or partially premixed components, e. g. components comprising compounds of the present invention and/or mixing partners as defined above, can be applied jointly (e.g. after tank mix) or consecutively.

Application Methods

The compounds of the present invention are suitable for use in protecting crops, plants, plant propagation materials, such as seeds, or soil or water, in which the plants are growing, from attack or infestation by animal pests. Therefore, the present invention also relates to a plant protection method, which comprises contacting crops, plants, plant propagation materials, such as seeds, or soil or water, in which the plants are growing, to be protected from attack or infestation by animal pests, with a pesticidally effective amount of a compound of the present invention.

The compounds of the present invention are also suitable for use in combating or controlling animal pests. Therefore, the present invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the present invention.

The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds/compositions directly on the animal pest or plant—typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The term “crop” refers to both, growing and harvested crops.

The term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevie); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “plant” is to be understood as including wild type plants and plants, which have been modified by either conventional breeding, or mutagenesis or genetic engineering, or by a combination thereof.

Plants, which have been modified by mutagenesis or genetic engineering, and are of particular commercial importance, include alfalfa, rapeseed (e.g. oilseed rape), bean, carnation, chicory, cotton, eggplant, eucalyptus, flax, lentil, maize, melon, papaya, petunia, plum, poplar, potato, rice, soybean, squash, sugar beet, sugarcane, sunflower, sweet pepper, tobacco, tomato, and cereals (e.g. wheat), in particular maize, soybean, cotton, wheat, and rice. In plants, which have been modified by mutagenesis or genetic engineering, one or more genes have been mutagenized or integrated into the genetic material of the plant. The one or more mutagenized or integrated genes are preferably selected from pat, epsps, cry1Ab, bar, cry1Fa2, cry1Ac, cry34Ab1, cry35AB1, cry3A, cryF, cry1F, mcry3a, cry2Ab2, cry3Bb1, cry1A.105, dfr, barnase, vip3Aa20, barstar, als, bxn, bp40, asn1, and ppo5. The mutagenesis or integration of the one or more genes is performed in order to improve certain properties of the plant. Such properties, also known as traits, include abiotic stress tolerance, altered growth/yield, disease resistance, herbicide tolerance, insect resistance, modified product quality, and pollination control. Of these properties, herbicide tolerance, e.g. imidazolinone tolerance, glyphosate tolerance, or glufosinate tolerance, is of particular importance. Several plants have been rendered tolerant to herbicides by mutagenesis, for example Clearfield® oilseed rape being tolerant to imidazolinones, e.g. imazamox. Alternatively, genetic engineering methods have been used to render plants, such as soybean, cotton, corn, beets and oil seed rape, tolerant to herbicides, such as glyphosate and glufosinate, some of which are commercially available under the trade names RoundupReady® (glyphosate) and LibertyLink® (glufosinate). Furthermore, insect resistance is of importance, in particular lepidopteran insect resistance and coleopteran insect resistance. Insect resistance is typically achieved by modifying plants by integrating cry and/or vip genes, which were isolated from Bacillus thuringiensis (Bt), and code for the respective Bt toxins. Genetically modified plants with insect resistance are commercially available under trade names including WideStrike®, Bollgard®, Agrisure®, Herculex®, YieldGard®, Genuity®, and Intacta®. Plants may be modified by mutagenesis or genetic engineering either in terms of one property (singular traits) or in terms of a combination of properties (stacked traits). Stacked traits, e.g. the combination of herbicide tolerance and insect resistance, are of increasing importance. In general, all relevant modified plants in connection with singular or stacked traits as well as detailed information as to the mutagenized or integrated genes and the respective events are available from websites of the organizations “International Service for the Acquisition of Agri-biotech Applications (ISAAA)” (http://www.isaaa.org/gmapprovaldatabase) and “Center for Environmental Risk Assessment (CERA)” (http://cera-gmc.org/GMCropDatabase).

It has surprisingly been found that the pesticidal activity of the compounds of the present invention may be enhanced by the insecticidal trait of a modified plant. Furthermore, it has been found that the compounds of the present invention are suitable for preventing insects to become resistant to the insecticidal trait or for combating pests, which already have become resistant to the insecticidal trait of a modified plant. Moreover, the compounds of the present invention are suitable for combating pests, against which the insecticidal trait is not effective, so that a complementary insecticidal activity can advantageously be used.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the present invention are particularly suitable for use in the treatment of seeds in order to protect the seeds from insect pests, in particular from soil-living insect pests, and the resulting seedling's roots and shoots against soil pests and foliar insects. The present invention therefore also relates to a method for the protection of seeds from insects, in particular from soil insects, and of the seedling's roots and shoots from insects, in particular from soil and foliar insects, said method comprising treating the seeds before sowing and/or after pregermination with a compound of the present invention. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenesis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of the compounds of the present invention for seed treatment usually comprise from 0.1 to 80% by weight (1 to 800 g/1) of the active ingredient, from 0.1 to 20% by weight (1 to 200 g/1) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the present invention, or an agriculturally useful salt thereof, as defined herein. The amount of the compound of the present invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of the present invention may also be used for improving the health of a plant. Therefore, the present invention also relates to a method for improving plant health by treating a plant, plant propagation material and/or the locus where the plant is growing or is to grow with an effective and non-phytotoxic amount of a compound of the present invention.

As used herein “an effective and non-phytotoxic amount” means that the compound is used in a quantity which allows to obtain the desired effect but which does not give rise to any phytotoxic symptom on the treated plant or on the plant grown from the treated propagule or treated soil.

The terms “plant” and “plant propagation material” are defined above.

“Plant health” is defined as a condition of the plant and/or its products which is determined by several aspects alone or in combination with each other such as yield (for example increased biomass and/or increased content of valuable ingredients), quality (for example improved content or composition of certain ingredients or shelf life), plant vigour (for example improved plant growth and/or greener leaves (“greening effect”), tolerance to abiotic (for example drought) and/or biotic stress (for example disease) and production efficiency (for example, harvesting efficiency, processability).

The above identified indicators for the health condition of a plant may be interdependent and may result from each other. Each indicator is defined in the art and can be determined by methods known to a skilled person.

The compounds of the invention are also suitable for use against non-crop insect pests. For use against said non-crop pests, compounds of the present invention can be used as bait composition, gel, general insect spray, aerosol, as ultra-low volume application and bed net (impregnated or surface applied). Furthermore, drenching and rodding methods can be used.

As used herein, the term “non-crop insect pest” refers to pests, which are particularly relevant for non-crop targets, such as ants, termites, wasps, flies, ticks, mosquitos, crickets, or cockroaches.

The bait can be a liquid, a solid or a semisolid preparation (e.g. a gel). The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature (e.g. http://www.pherobase.com), and are known to those skilled in the art.

For use in bait compositions, the typical content of active ingredient is from 0.001 weight % to 15 weight %, desirably from 0.001 weight % to 5% weight % of active compound.

Formulations of the compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents, furthermore auxiliaries such as emulsifiers, perfume oils, if appropriate stabilizers, and, if required, propellants.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The compounds of the present invention and its compositions can be used for protecting wooden materials such as trees, board fences, sleepers, frames, artistic artifacts, etc. and buildings, but also construction materials, furniture, leathers, fibers, vinyl articles, electric wires and cables etc. from ants and/or termites, and for controlling ants and termites from doing harm to crops or human being (e.g. when the pests invade into houses and public facilities).

Customary application rates in the protection of materials are, for example, from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

Pests

The compounds of the present invention are especially suitable for efficiently combating animal pests such as arthropods, gastropods and nematodes including but not limited to: insects from the order of Lepidoptera, for example Achroia grisella, Acleris spp. such as A. fimbriana, A. gloverana, A. variana; Acrolepiopsis assectella, Acronicta major, Adoxophyes spp. such as A. cyrtosema, A. orana; Aedia leucomelas, Agrotis spp. such as A. exclamationis, A. fucosa, A. ipsilon, A. orthogoma, A. segetum, A. subterranea; Alabama argillacea, Aleurodicus dispersus, Alsophila pometaria, Ampelophaga rubiginosa, Amyelois transitella, Anacampsis sarcitella, Anagasta kuehniella, Anarsia lineatella, Anisota senatoria, Antheraea pernyi, Anticarsia (=Thermesia) spp. such as A. gemmatalis; Apamea spp., Aproaerema modicella, Archips spp. such as A. argyrospila, A. fuscocupreanus, A. rosana, A. xyloseanus; Argyresthia conjugella, Argyroploce spp., Argyrotaenia spp. such as A. velutinana; Athetis mindara, Austroasca viridigrisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp. such as C. murinana, C. podana; Cactoblastis cactorum, Cadra cautella, Calingo braziliensis, Caloptilis theivora, Capua reticulana, Carposina spp. such as C. niponensis, C. sasakii; Cephus spp., Chaetocnema aridula, Cheimatobia brumata, Chilo spp. such as C. indicus, C. suppressalis, C. partellus; Choreutis pariana, Choristoneura spp. such as C. conflictana, C. fumiferana, C. longicellana, C. murinana, C. occidentaiis, C. rosaceana; Chrysodeixis (=Pseudoplusia) spp. such as C. eriosoma, C. inciudens; Cirphis unipuneta, Ciysia ambigueiia, Cnaphaiocerus spp., Cnaphaiocrocis medinaiis, Cnephasia spp., Cochylis hospes, Coleophora spp., Colias eurytheme, Conopomorphaspp., Conotracheius spp., Copitarsia spp., Corcyra cephaionica, Crambus caliginosellus, Crambus teterrellus, Crocidosema (=Epinotia) aporema, Cydalima (=Diaphania) perspectalis, Cydia (=Carpocapsa) spp. such as C. pomonella, C. latiferreana; Dalaca noctuides, Datana integerrima, Dasychira pinicola, Dendrolimus spp. such as D. pini, D. spectabilis, D. sibiricus; Desmia funeralis, Diaphania spp. such as D. nitidalis, D. hyalinata; Diatraea grandioselia, Diatraea saccharalis, Diphthera festiva, Earias spp. such as E. insulana, E. vittella; Ecdytolopha aurantianu, Egira (=Xylomyges) curiaiis, Elasmopaipus linosellus, Eidana saccharina, Endopiza viteana, Ennomos subsignaria, Eoreuma loftini, Ephestia spp. such as E. cautella, E. eiutella, E. kuehniella; Epinotia aporema, Epiphyas postvittana, Erannis tiliaria, Erionota thrax, Etiella spp., Eulia spp., Eupoecilia ambigueiia, Euproctis chrysorrhoea, Euxoa spp., Evetria bouliana, Faronta albilinea, Feltia spp. such as F. subterranean; Galleria mellonella, Gracillaria spp., Graphollta spp. such as G. funebrana, G. moiesta, G. inopinata; Halysidota spp., Harrisina americana, Hedylepta spp., Helicoverpa spp. such as H. armigera (=Heliothis armigera), H. zea (=Heliothis zea); Heliothis spp. such as H. assulta, H. subflexa, H. virescens; Hellula spp. such as H. undalis, H. rogatalis; Helocoverpa gelotopoeon, Hemileuca oliviae, Herpetogramma licarsisalis, Hibernia defoliaria, Hofmannophila pseudospretella, Homoeosoma eiectellum, Homona magnanima, Hypena scabra, Hyphantria cunea, Hyponomeuta padella, Hyponomeuta malinellus, Kakivoria flavofasciata, Keiferia lycopersicella, Lambdiria fiscellaria fiscellaria, Lambdiria fiscellaria lugubrosa, Lamprosema iridicata, Laspeyresia molesta, Leguminivora glycinivorella, Lerodea eufala, Leucinodes orbonalis, Leucoma salicis, Leucoptera spp. such as L. coffeella, L. scitella; Leuminivora lycinivorella, Lithocolletis blancardella, Lithophane anternnata, Llattia octo (=Amyna axis), Lobesia botraria, Lophocampa spp., Loxagrotis albicosta, Loxostege spp. such as L. sticticalis, L. cereralis; Lymantria spp. such as L. dispar, L. monacha; Lyonetia cierkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quiriquemaculata, M. sexta; Marasmia spp, Marmara spp., Maruca testulalis, Megalopyge lariata, Melanchra picta, Melanitis leda, Mocis spp. such as M. lapites, M. repanda; Mocis latipes, Monochroa fragariae, Mythimna separata, Nemapogon cloacella, Neoleucinodes elegantalis, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp. such as O. nubilalis; Oulema oryzae, Paleacrita vernata, Panolis flammea, Parnara spp., Papaipema nebris, Papilio cresphontes, Paramyelois transitella, Paranthrene regalis, Paysandisia archon, Pectinophora spp. such as P. gossypiella; Peridroma saucia, Perileucoptera spp., such as P. coffeella; Phalera bucephala, Phryganidia californica, Phthorimaea spp. such as P. operculella; Phyllocnistis citrella, Phyllonorycter spp. such as P. blancardella, P. crataegella, P. issikii, P. ringoniella; Pieris spp. such as P. brassicae, P. rapae, P. napi; Pilocrocis tripunctata, Plathypena scabra, Platynota spp. such as P. flavedana, P. idaeusalis, P. stultana; Platyptilia carduidactyla, Plebejus argus, Plodia interpunctella, Plusia spp, Plutella maculipennis, Plutella xylostella, Pontia protodica, Prays spp., Prodenia spp., Proxenus lepigone, Pseudaletia spp. such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp. such as S. incertulas, S. innotata; Scotia segetum, Sesamia spp. such as S. inferens, Seudyra subflava, Sitotroga cerealella, Sparganothis pilleriana, Spilonota lechriaspis, S. ocellana, Spodoptera (=Lamphygma) spp. such as S. cosmoides, S. eridania, S. exigua, S. frugiperda, S. latisfascia, S. littoralis, S. litura, S. omithogalli; Stigmella spp., Stomopteryx subsecivella, Strymon bazochii, Sylepta derogata, Synanthedon spp. such as S. exitiosa, Tecia solanivora, Telehin licus, Thaumatopoea pityocampa, Thaumatotibia (=Cryptophlebia) leucotreta, Thaumetopoea pityocampa, Thecla spp., Theresimima ampelophaga, Thyrinteina spp, Tildenia inconspicuella, Tinea spp. such as T. cloacella, T. pellionella; Tineola bisselliella, Tortrx spp. such as T. viridana; Trichophaga tapetzella, Trichoplusia spp. such as T. ni; Tuta (=Scrobipalpula) absoluta, Udea spp. such as U. rubigalis, U. rubigalis; Virachola spp., Yponomeuta padella, and Zeiraphera canadensis;

insects from the order of Coleoptera, for example Acalymma vittatum, Acanthoscehdes obtectus, Adoretus spp., Agelastica alni, Agrilus spp. such as A. anxius, A. planipennis, A. sinuatus; Agriotes spp. such as A. fuscicollis, A. lineatus, A. obscurus; Alphitobius diaperinus, Amphimallus solstitialis, Anisandrus dispar, Anisoplia austriaca, Anobium punctatum, Anomala corpulenta, Anomala rufocuprea, Anoplophora spp. such as A. glabripennis; Anthonomus spp. such as A. eugenii, A. grandis, A. pomorum; Anthrenus spp., Aphthona euphoridae, Apion spp., Apogonia spp., Athous haemorrhoidalis, Atomaria spp. such as A. linearis; Attagenus spp., Aulacophora femoralis, Blastophagus piniperda, Blitophaga undata, Bruchidius obtectus, Bruchus spp. such as B. lentis, B. pisorum, B. rufimanus; Byctiscus betulae, Callidiellum rufipenne, Callopistria floridensis, Callosobruchus chinensis, Cameraria ohridella, Cassida nebulosa, Cerotoma trifurcata, Cetonia aurata, Ceuthorhynchus spp. such as C. assimilis, C. napi; Chaetocnema tibialis, Cleonus mendicus, Conoderus spp. such as C. vespertinus; Conotrachelus nenuphar, Cosmopolites spp., Costelytra zealandica, Crioceris asparagi, Cryptolestes ferrugineus, Cryptorhynchus lapathi, Ctenicera spp. such as C. destructor; Curculio spp., Cylindrocopturus spp., Cyclocephala spp., Dactylispabalyi, Dectestexanus, Dermestes spp., Diabrotica spp. such as D. undecimpunctata, D. speciosa, D. longicornis, D. semipunctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. similaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp. such as H. brunneipennis, H. postica; Hypomeces squamosus, Hypothenemus spp., Ips typographus, Lachnosterna consanguinea, Lasioderma serricorne, Latheticus oryzae, Lathridius spp., Lema spp. such as L. bilineata, L. melanopus; Leptinotarsa spp. such as L. decemlineata; Leptispapygmaea, Limonius californicus, Lissorhoptrus oryzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus; Melolontha spp. such as M. hippocastani, M. melolontha; Metamasius hemipterus, Microtheca spp., Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp. such as P. brassicae, P. cochleariae; Phoracantha recurva, Phyllobius pyri, Phyllopertha horticola, Phyllophaga spp. such as P. heller; Phyllotreta spp. such as P. chrysocephala, P. nemorum, P. striolata, P. vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. billineatus, R. ferrugineus, R. palmarum, R. phoenicis, R. vulneratus; Saperda candida, Scolytus schevyrewi, Scyphophorus acupunctatus, Sitona lineatus, Sitophilus spp. such as S. granaria, S. oryzae, S. zeamais; Sphenophorus spp. such as S. levis; Stegobium paniceum, Sternechus spp. such as S. subsignatus; Strophomorphus ctenotus, Symphyletes spp., Tanymecus spp., Tenebrio molitor, Tenebrioides mauretanicus, Tribolium spp. such as T. castaneum; Trogoderma spp., Tychiusspp., Xylotrechus spp. such as X. pyrrhoderus; and, Zabrus spp. such as Z. tenebrioides;

insects from the order of Diptera for example Aedes spp. such as A. aegypti, A. albopictus, A. vexans; Anastrepha ludens, Anopheles spp. such as A. albimanus, A. crucians, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria; Chrysops atlanticus, Chrysops discalis, Chrysops silacea, Cochliomyia spp. such as C. hominivorax; Contarinia spp. such as C. sorghicola; Cordylobia anthropophaga, Culex spp. such as C. nigripalpus, C. pipiens, C. quinquefasciatus, C. tarsalis, C. tritaeniorhynchus; Culicoides furens, Culiseta inornata, Culiseta melanura, Cuterebra spp., Dacus cucurbitae, Dacus oleae, Dasineura brassicae, Dasineura oxycoccana, Delia spp. such as D. antique, D. coarctata, D. platura, D. radicum; Dermatobia hominis, Drosophila spp. such as D. suzukii, Fannia spp. such as F. canicularis; Gastraphilus spp. such as G. intestinalis; Geomyza tipunctata, Glossina spp. such as G. fuscipes, G. morsitans, G. palpalis, G. tachinoides; Haematobia irritans, Haplodiplosis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii; Lucilia spp. such as L. caprina, L. cuprina, L. sericata; Lycoria pectoralis, Mansonia titillanus, Mayetiola spp. such as M. destructor; Musca spp. such as M. autumnalis, M. domestica; Muscina stabulans, Oestrus spp. such as O. ovis; Opomyza florum, Oscinella spp. such as O. frit; Orseolia oryzae, Pegomya hysocyami, Phlebotomus argentipes, Phorbia spp. such as P. antiqua, P. brassicae, P. coarctata; Phytomyza gymnostoma, Prosimulium mixtum, Psila rosae, Psorophora columbiae, Psorophora discolor, Rhagoletis spp. such as R. cerasi, R. cingulate, R. indifferens, R. mendax, R. pomonella; Rivellia quadrifasciata, Sarcophaga spp. such as S. haemorrhoidalis; Simulium vittatum, Sitodiplosis mosellana, Stomoxys spp. such as S. calcitrans; Tabanus spp. such as T. atratus, T. bovinus, T. lineola, T. similis; Tannia spp., Thecodiplosis japonensis, Tipula oleracea, Tipula paludosa, and Wohlfahrtia spp;

insects from the order of Thysanoptera for example, Baiothrips biformis, Dichromothrips corbetti, Dichromothrips ssp., Echinothrips americanus, Enneothrips flavens, Frankliniella spp. such as F. fusca, F. occidentalis, F. tritici; Heliothrips spp., Hercinothrips femoralis, Kakothrips spp., Microcephalothrips abdominalis, Neohydatothrips samayunkur, Pezothrips kellyanus, Rhipiphorothrips cruentatus, Scirtothrips spp. such as S. citri, S. dorsalis, S. perseae; Stenchaetothrips spp, Taeniothrips cardamon, Taeniothrips inconsequens, Thrips spp. such as T. imagines, T. hawaiiensis, T. oryzae, T. palmi, T. parvispinus, T. tabaci;

insects from the order of Hemiptera for example, Acizzia jamatonica, Acrosternum spp. such as A. hiare; Acyrthosipon spp. such as A. onobrychis, A. pisum; Adelges laricis, Adelges tsugae, Adelphocoris spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrxus spp., Amrasca spp., Anasa tristis, Antestiopsis spp., Anuraphis cardui, Aonidiella spp., Aphanostigma piri, Aphidula nasturtii, Aphis spp. such as A. craccivora, A. fabae, A. forbesi, A. gossypii, A. grossulariae, A. maidiradicis, A. pomi, A. sambuci, A. schneideri, A. spiraecola; Arboridia apicalis, Arilus critatus, Aspidiella spp., Aspidiotus spp., Atanus spp., Aulacaspis yasumatsui, Aulacorthum solani, Bactericera cockerelli (Paratrioza cockereli), Bemisia spp. such as B. argentifolii, B. tabaci (Aleurodes tabaci); Blissus spp. such as B. leucopterus; Brachycaudus spp. such as B. cardui, B. helichrysi, B. persicae, B. prunicola; Brachycolus spp., Brachycorynella asparagi, Brevicoryne brassicae, Cacopsylla spp. such as C. fulguralis, C. pyricola (Psylla pin); Calligypona marginata, Calocoris spp., Campylomma livida, Capitophorus horni, Carneocephala fulgida, Cavelerius spp., Ceraplastes spp., Ceratovacuna lanigera, Ceroplastes ceriferus, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as C. hemipterus, C. lectularius; Coccomytilus halli, Coccus spp. such as C. hesperidum, C. pseudomagnoliarum; Corythucha arcuata, Creontiades dilutus, Cryptomyzus ribis, Chrysomphalus aonidum, Cryptomyzus ribis, Ctenarytaina spatulata, Cyrtopeltis notatus, Dalbulus spp., Dasynus piperis, Dialeurodes spp. such as D. citrifolii; Dalbulus maidis, Diaphorina spp. such as D. citri; Diaspis spp. such as D. bromeliae; Dichelops furcatus, Diconocoris hewett, Doralis spp., Dreyfusia nordmannianae, Dreyfusia piceae, Drosicha spp., Dysaphis spp. such as D. plantaginea, D. pyn, D. radicola; Dysaulacorthum pseudosolan, Dysdercus spp. such as D. cingulatus, D. intermedius; Dysmicoccus spp., Edessa spp., Geocoris spp., Empoasca spp. such as E. fabae, E. solana; Epidiaspis leperii, Eriosoma spp. such as E. lanigerum, E. pyricola; Erythroneura spp., Eurygaster spp. such as E. integriceps; Euscelis bilobatus, Euschistus spp. such as E. heros, E. impictiventris, E. servus; Fiorinia theae, Geococcus coffeae, Glycaspis brimblecombei, Halyomorpha spp. such as H. halys; Heliopeltis spp., Homalodisca vitrpennis (=H. coagulata), Horcias nobilellus, Hyalopterus pruni, Hyperomyzus lactucae, Icerya spp. such as I. purchase; Idiocerus spp., Idioscopus spp., Laodelphax striatellus, Lecanium spp., Lecanoideus floccissimus, Lepidosaphes spp. such as L. ulm; Leptocorisa spp., Leptoglossus phyllopus, Lipaphis erysimi, Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbae; Macrosteles quadrilineatus, Mahanarva fimbriolata, Megacopta cribraria, Megoura viciae, Melanaphis pyrarius, Melanaphis sacchari, Melanocallis (=Tinocallis) caryaefolae, Metcaiella spp., Metopolophium dirhodum, Monellia costalis, Monelliopsis pecanis, Myzocallis coryl, Murgantia spp., Myzus spp. such as M. ascalonicus, M. ceras, M. nicotianae, M. persicae, M. varians; Nasonovia ribis-ngri, Neotoxoptera formosana, Neomegalotomus spp, Nephotettix spp. such as N. malayanus, N. ngropictus, N. parvus, N. vrescens; Nezara spp. such as N. viridula; Nilaparvata lugens, Nysius huttoni, Oebalus spp. such as O. pugnax; Oncometopia spp., Orthezia praelonga, Oxycaraenus hyalinipennis, Parabemisia myricae, Parlatoria spp., Parthenolecanium spp. such as P. corni, P. persicae; Pemphigus spp. such as P. bursarius, P. populivenae; Peregrinus maidis, Perkinsiella saccharicida, Phenacoccus spp. such as P. aceris, P. gossypi; Phloeomyzus passerinii, Phorodon humuli, Phylloxera spp. such as P. devastatrx, Piesma quadrata, Piezodorus spp. such as P. guildinii; Pinnaspis aspidistrae, Planococcus spp. such as P. citri, P. ficus; Prosapia bicincta, Protopulvnara pyrforms, Psallus seratus, Pseudacysta persea, Pseudaulacaspis pentagona, Pseudococcus spp. such as P. comstocki; Psylla spp. such as P. mali; Pteromalus spp., Pulvinaria amygdal, Pyrilla spp., Quadraspidiotus spp., such as Q. perniciosus; Quesada gigas, Rastrococcus spp., Reduvius senils, Rhzoecus amercanus, Rhodnius spp., Rhopalomyzus ascalonicus, Rhopalosiphum spp. such as R. pseudobrassicas, R. insertum, R. maidis, R. padi; Sagatodes spp., Sahlbergella singularis, Saissetia spp., Sappaphis mala, Sappaphis mali, Scaptocoris spp., Scaphoides titanus, Schzaphs gramnum, Schizoneura lanuginosa, Scotinophora spp., Selenaspidus articulatus, Sitobion avenae, Sogata spp., Sogatella furcifera, Solubea insularis, Spissistilus festinus (=Stictocephala festina), Stephanitis nash, Stephants pyrioides, Stephantis takeyai, Tenalaphara malayensis, Tetraleurodes perseae, Theroaphis maculate, Thyanta spp. such as T. accerra, T. perditor Tibraca spp., Tomaspis spp., Toxoptera spp. such as T. aurantii; Trialeurodes spp. such as T. abutilonea, T. ricini, T. vaporariorum; Triatoma spp., Trioza spp., Typhlocyba spp., Unaspis spp. such as U. citri, U. yanonensis; and Viteus vitifolii,

Insects from the order Hymenoptera for example Acanthomyops interjectus, Athalia rosae, Atta spp. such as A. capiguara, A. cephalotes, A. cephalotes, A. laevigata, A. robusta, A. sexdens, A. texana, Bombus spp., Brachymyrmex spp., Camponotus spp. such as C. floridanus, C. pennsylvanicus, C. modoc; Cardiocondyla nuda, Chalibion sp, Crematogaster spp., Dasymutilla occidentalis, Diprion spp., Dolichovespula maculata, Dorymyrmex spp., Dryocosmus kuriphilus, Formica spp., Hoplocampa spp. such as H. minuta, H. testudinea; Iridomyrmex humilis, Lasius spp. such as L. niger, Linepithema humile, Liometopum spp., Leptocybe invasa, Monomorium spp. such as M. pharaons, Monomorium, Nylandria fulva, Pachycondyla chinensis, Paratrechina longicornis, Paravespula spp., such as P. germanica, P. pennsylvanica, P. vulgaris; Pheidole spp. such as P. megacephala; Pogonomyrmex spp. such as P. barbatus, P. californicus, Polistes rubiginosa, Prenolepis impairs, Pseudomyrmex gracilis, Schelipron spp., Sirex cyaneus, Solenopsis spp. such as S. geminata, S. invicta, S. molesta, S. richter, S. xyloni, Sphecius speciosus, Sphex spp., Tapinoma spp. such as T. melanocephalum, T. sessile; Tetramorium spp. such as T. caespitum, T. bicarinatum, Vespa spp. such as V. crabro; Vespula spp. such as V. squamosal; Wasmannia auropunctata, Xylocopa sp;

Insects from the order Orthoptera for example Acheta domesticus, Calliptamus italicus, Chortoicetes terminifera, Ceuthophilus spp., Diastrammena asynamora, Docostaurus maroccanus, Gryllotalpa spp. such as G. africana, G. gryllotalpa; Gryllus spp., Hieroglyphus daganensis, Kraussaria angulifera, Locusta spp. such as L. migratoria, L. pardalina; Melanoplus spp. such as M. bivittatus, M. femurrubrum, M. mexicanus, M. sanguinipes, M. spretus; Nomadacris septemfasciata, Oedaleus senegalensis, Scapteriscus spp., Schistocerca spp. such as S. americana, S. gregaria, Stemopelmatus spp., Tachycines asynamorus, and Zonozerus variegatus;

Pests from the Class Arachnida for example Acari, e.g. of the families Argasidae, Ixodidae and Sarcoptidae, such as Amblyomma spp. (e.g. A. americanum, A. variegatum, A. maculatum), Argas spp. such as A. persicu), Boophius spp. such as B. annulatus, B. decoloratus, B. microplus, Dermacentor spp. such as D. silvarum, D. anderson, D. variabilis, Hyalomma spp. such as H. truncatum, Ixodes spp. such as I. ricinus, I. rubicundus, I. scapularis, I. holocyclus, I. pacficus, Rhipicephalus sanguineus, Ornithodorus spp. such as O. moubata, O. herms, O. turicata, Ornithonyssus bacoti, Otobius megnin, Dermanyssus gallinae, Psoroptes spp. such as P. ovis, Rhipicephalus spp. such as R. sanguineus, R. appendiculatus, Rhipicephalus everts, Rhizoglyphus spp., Sarcoptes spp. such as S. Scabier; and Family Eriophyidae including Aceria spp. such as A. sheldoni, A. anthocoptes, Acallitus spp., Aculops spp. such as A. lycopersici, A. pelekassi Aculus spp. such as A. schlechtendali; Colomerus vitis, Epitrimerus pyri, Phyllocoptruta oleivora; Eriophytes ribis and Eriophyes spp. such as Eriophyes sheldoni Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp. such as B. phoenicis; Family Tetranychidae including Eotetranychus spp., Eutetranychus spp., Oligonychus spp., Petrobia latens, Tetranychus spp. such as T. cinnabarinus, T. evans, T. kanzawa, T. pacificus, T. phaseulus, T. telarius and T. urticae; Bryobia praetiosa; Panonychus spp. such as P. ulmi, P. citri Metatetranychus spp. and Oligonychus spp. such as O. pratensis, O. perseae, Vasates lycopersici; Raorella indica, Family Carpoglyphidae including Carpoglyphus spp.; Penthaleidae spp. such as Halotydeus destructor, Family Demodicidae with species such as Demodex spp.; Family Trombicidea including Trombicula spp.; Family Macronyssidae including Ornothonyssus spp.; Family Pyemotidae including Pyemotes tritici Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa;

Pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. schachtii, H. trifoii; Seed gall nematodes, Anguina spp.; Stem and foliar nematodes, Aphelenchoides spp. such as A. besseyi; Sting nematodes, Belonolaimus spp. such as B. longicaudatus; Pine nematodes, Bursaphelenchus spp. such as B. lignicolus, B. xylophilus; Ring nematodes, Criconema spp., Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Heliocotylenchus multicinctus; Sheath and sheathoid nematodes, Hemicycliophora spp. and Hemicriconemoides spp.; Hirshmanniella spp.; Lance nematodes, Hoploaimus spp.; False rootknot nematodes, Nacobbus spp.; Needle nematodes, Longidorus spp. such as L. elongatus; Lesion nematodes, Pratylenchus spp. such as P. brachyurus, P. neglectus, P. penetrans, P. curvitatus, P. goodeyi; Burrowing nematodes, Radopholus spp. such as R. similis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. primitivus; Paratrichodorus spp. such as P. minor; Stunt nematodes, Tylenchorhynchus spp. such as T. claytoni, T. dubius; Citrus nematodes, Tylenchulus spp. such as T. semipenetrans; Dagger nematodes, Xiphinema spp.; and other plant parasitic nematode species;

Insects from the order Isoptera for example Calotermes flavicolis, Coptotermes spp. such as C. formosanus, C. gestroi, C. acinaciformis; Cornitermes cumulans, Cryptotermes spp. such as C. brevis, C. cavifrons; Globitermes sulfureus, Heterotermes spp. such as H. aureus, H. longiceps, H. tenuis; Leucotermes flavpes, Odontotermes spp., Incisitermes spp. such as I. minor, I. snyder; Marginitermes hubbardi, Mastotermes spp. such as M. darwiniensis Neocapritermes spp. such as N. opacus, N. parvus Neotermes spp., Procornitermes spp., Zootermopsis spp. such as Z. angusticollis, Z. nevadensis, Reticulitermes spp. such as R. hesperus, R. tibialis, R. speratus, R. flavipes, R. grassei, R. lucifugus, R. santonensis, R. virginicus; Termes natalensis,

Insects from the order Blattaria for example Blatta spp. such as B. orientalis, B. lateralis; Blattella spp. such as B. asahinae, B. germanica; Leucophaea maderae, Panchlora nivea, Perplaneta spp. such as P. americana, P. australasiae, P. brunnea, P. fuligginosa, P. japonica; Supella longipalpa, Parcoblatta pennsylvanica, Eurycotis flordana, Pycnoscelus surinamensis,

Insects from the order Siphonoptera for example Cediopsylla simples, Ceratophyllus spp., Ctenocephalides spp. such as C. felis, C. canis, Xenopsylla cheopis, Pulex irritans, Trichodectes canis, Tunga penetrans, and Nosopsyllus fasciatus,

Insects from the order Thysanura for example Lepisma saccharna, Ctenolepisma urbana, and Thermobia domestica,

Pests from the class Chilopoda for example Geophilus spp., Scutigera spp. such as Scutigera coleoptrata;

Pests from the class Diplopoda for example Blaniulus guttulatus, Julus spp., Narceus spp.,

Pests from the class Symphyla for example Scutigerella immaculata,

Insects from the order Dermaptera, for example Forficula aurculara,

Insects from the order Collembola, for example Onychiurus spp., such as Onychiurus armatus,

Pests from the order Isopoda for example, Armadillidium vulgare, Oniscus asellus, Porcellio scaber,

Insects from the order Phthiraptera, for example Damalinia spp., Pediculus spp. such as Pediculus humanus capitis, Pediculus humanus corporis, Pediculus humanus humanus; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp.,

Examples of further pest species which may be controlled by compounds of formula (I) include: from the Phylum Mollusca, class Bivalvia, for example, Dreissena spp.; class Gastropoda, for example, Arion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti.

Animal Health

The compounds of the present invention are suitable for use in treating or protecting animals against infestation or infection by parasites. Therefore, the present invention also relates to the use of a compound of the present invention for the manufacture of a medicament for the treatment or protection of animals against infestation or infection by parasites. Furthermore, the present invention relates to a method of treating or protecting animals against infestation and infection by parasites, which comprises orally, topically or parenterally administering or applying to the animals a parasiticidally effective amount of a compound of the present invention.

The present invention also relates to the non-therapeutic use of compounds of the present invention for treating or protecting animals against infestation and infection by parasites. Moreover, the present invention relates to a non-therapeutic method of treating or protecting animals against infestation and infection by parasites, which comprises applying to a locus a parasiticidally effective amount of a compound of the present invention.

The compounds of the present invention are further suitable for use in combating or controlling parasites in and on animals. Furthermore, the present invention relates to a method of combating or controlling parasites in and on animals, which comprises contacting the parasites with a parasitically effective amount of a compound of the present invention.

The present invention also relates to the non-therapeutic use of compounds of the present invention for controlling or combating parasites. Moreover, the present invention relates to a non-therapeutic method of combating or controlling parasites, which comprises applying to a locus a parasiticidally effective amount of a compound of the present invention.

The compounds of the present invention can be effective through both contact (via soil, glass, wall, bed net, carpet, blankets or animal parts) and ingestion (e.g. baits). Furthermore, the compounds of the present invention can be applied to any and all developmental stages.

The compounds of the present invention can be applied as such or in form of compositions comprising the compounds of the present invention.

The compounds of the present invention can also be applied together with a mixing partner, which acts against pathogenic parasites, e.g. with synthetic coccidiosis compounds, polyetherantibiotics such as Amprolium, Robenidin, Toltrazuril, Monensin, Salinomycin, Maduramicin, Lasalocid, Narasin or Semduramicin, or with other mixing partners as defined above, or in form of compositions comprising said mixtures.

The compounds of the present invention and compositions comprising them can be applied orally, parenterally or topically, e.g. dermally. The compounds of the present invention can be systemically or non-systemically effective.

The application can be carried out prophylactically, therapeutically or non-therapeutically. Furthermore, the application can be carried out preventively to places at which occurrence of the parasites is expected.

As used herein, the term “contacting” includes both direct contact (applying the compounds/compositions directly on the parasite, including the application directly on the animal or excluding the application directly on the animal, e.g. at it's locus for the latter) and indirect contact (applying the compounds/compositions to the locus of the parasite). The contact of the parasite through application to its locus is an example of a non-therapeutic use of the compounds of the present invention.

The term “locus” means the habitat, food supply, breeding ground, area, material or environment in which a parasite is growing or may grow outside of the animal.

As used herein, the term “parasites” includes endo- and ectoparasites. In some embodiments of the present invention, endoparasites can be preferred. In other embodiments, ectoparasites can be preferred. Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus; cockroaches (Blattaria-Blattodea), e.g. Blattella germanica, Blattella asahinae, Periplaneta americana, Periplaneta japonica, Periplaneta brunnea, Perplaneta fuligginosa, Perplaneta australasiae, and Blatta orientalis; flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis; lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus; ticks and parasitic mites (Parasitiformes): ticks (Ixodida), e.g. Ixodes scapularis, Ixodes holocyclus, Ixodes pacificus, Rhiphicephalus sanguineus, Dermacentor anderson, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae; Actinedida (Prostigmata) und Acaridida (Astigmata), e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp; Bugs (Heteropterida): Cimex lectularius, Cimex hemipterus, Reduvius senilis, Triatoma spp., Rhodnius ssp., Panstrongylus ssp., and Arilus critatus; Anoplurida, e.g. Haematopinus spp., Linognathus spp., Pediculus spp., Phtirus spp., and Solenopotes spp.; Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp.; Roundworms Nematoda: Wipeworms and Trichinosis (Trichosyringida), e.g. Trichinellidae (Trichinella spp.), (Trichuridae) Trichuris spp., Capillaria spp.; Rhabditida, e.g. Rhabditis spp., Strongyloides spp., Helicephalobus spp.; Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp., Aleurostrongylus abstrusus, and Dioctophyma renale; Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi; Camallanida, e.g. Dracunculus medinensis (guinea worm); Spirurida, e.g. Thelazia spp., Wuchereria spp., Brugia spp., Onchocerca spp., Dirofilari spp. a, Dipetalonema spp., Setaria spp., Elaeophora spp., Spirocerca lupi, and Habronema Spp.; Thorny Headed Worms (Acanthocephala), e.g. Acanthocephalus spp., Macracanthorhynchus hirudinaceus and Oncicola spp.; Planarians (Plathelminthes): Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp.; Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

Generally, it is favorable to apply the compounds of the present invention in total amounts of 0.5 mg/kg to 100 mg/kg per day, preferably 1 mg/kg to 50 mg/kg per day.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further auxiliaries such as acids, bases, buffer salts, preservatives, and solubilizers. Suitable auxiliaries for injection solutions are known in the art. The solutions are filtered and filled sterile.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Gels are applied to or spread on the skin or introduced into body cavities. Gels are prepared by treating solutions which have been prepared as described in the case of the injection solutions with sufficient thickener that a clear material having an ointment-like consistency results. Suitable thickeners are known in the art.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added. Suitable such auxiliaries are known in the art.

Emulsions can be administered orally, dermally or as injections. Emulsions are either of the water-in-oil type or of the oil-in-water type. They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances. Suitable hydrophobic phases (oils), suitable hydrophilic phases, suitable emulsifiers, and suitable further auxiliaries for emulsions are known in the art.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers. Suitable suspending agents, and suitable other auxiliaries for suspensions including wetting agents are known in the art.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form. Suitable auxiliaries for this purpose are known in the art.

The compositions which can be used in the invention can comprise generally from about 0.001 to 95% of the compound of the present invention.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

Topical application may be conducted with compound-containing shaped articles such as collars, medallions, ear tags, bands for fixing at body parts, and adhesive strips and foils.

Generally it is favorable to apply solid formulations which release compounds of the present invention in total amounts of 10 mg/kg to 300 mg/kg, preferably 20 mg/kg to 200 mg/kg, most preferably 25 mg/kg to 160 mg/kg body weight of the treated animal in the course of three weeks.

### EXAMPLES

With appropriate modification of the starting materials, the procedures as described in the preparation examples below were used to obtain further compounds of formula I. The compounds obtained in this manner are listed in the table X that follows, together with physical data.

Compounds can be characterized e.g. by coupled High Performance Liquid Chromatography/mass spectrometry (HPLC/MS), or by ¹H-NMR and/or by their melting points.

### Example C-1:6-(2-chloroacetyl)-3-(2-chlorothiazol-5-yl)-7-methyl-2,3-dihydroimidazo[2,1-b]thiazol-4-ium-5-olate (C-1)

To a solution of 4-(2-chlorothiazol-5-yl)-N-methyl-4,5-dihydrothiazol-2-amine (0.100 g) (known from WO 2014/167084) in 1,4-dioxane (3 ml) was added chloroacetic acid (0.081 g) followed by 2-chloroacetic anhydride (0.146 g). The reaction mixture was stirred under reflux for 16 h. Then, the reaction mixture was cooled down to 23° C. and carefully quenched with Na₂CO₃ (10% water solution). After extraction with DCM, the combined organic phases were washed with Na₂CO₃ (10% water solution), dried over Na₂SO₄ and concentrated in vacuum. The crude product was purified using column chromatography, providing the title compound (0.03 g).

¹H NMR (500 MHz, Chloroform-d) δ 7.72 (s, 1H); 6.80 (d, 1H); 4.69-4.66 (m, 1H); 4.45-4.42 (m, 1H); 3.95-3.93 (m, 1H); 3.89-3.88 (m, 1H); 3.37 (s, 3H).

Biological Data:

Green Peach Aphid (Myzus persicae):

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the 30 aphid diet, using a custom built pipetter, at two replications.

After application, 5-8 adult aphids were placed on the artificial membrane inside the microtiter plate wells. The aphids were then allowed to suck on the treated aphid diet and incubated at about 23˜1° C. and about 50˜5% relative humidity for 3 days. Aphid mortality and fecundity was then visually assessed.

In this test, compound C-1 at 2500 ppm showed over 75% mortality in comparison with untreated controls.

Vetch Aphid (Megoura viciae):

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications. After application, the leaf disks were air-dried and 5-8 adult aphids placed on the leaf disks inside the microtiter plate wells. The aphids were then allowed to suck on the treated leaf disks and incubated at about 23+1° C. and about 50+5% relative humidity for 5 days. Aphid mortality and fecundity was then visually assessed.

In this test, compound C-1 at 2500 ppm showed over 75% mortality in comparison with untreated controls.

1-15. (canceled)

16. A compound of formula (I) 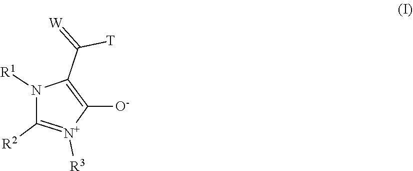
wherein
W is O, and
T is R⁵, OR⁶, —N(R⁷)(R⁸) or —N(R^{7a})—N(R⁷)(R⁸);
or
W is S, and
T is —N(R⁷)(R⁸) or —N(R^{7a})—N(R⁷)(R⁸);
R¹ is NO₂, CN C₁-C₈-alkyl, C₂-C₈-alkenyl, C₂-C₈-alkynyl, C₃-C₁₀-cycloalkyl, C₄-C₁₀-cycloalkenyl, C₅-C₁₄-cycloalkylcycloalkyl or R¹ may form a three- to eleven-membered saturated, or partially unsaturated or aromatic carbo- or heterocyclic ring or ring system, which may contain 1 to 4 heteroatoms selected from N(R^{c})ₚ, O, and S, wherein S may be oxidized, and wherein the aforementioned groups and the carbo- or heterocyclic rings system may be unsubstituted, partially or fully substituted with Ra; or
R¹ is C(═O)R^{b}, C(═O)OR^{e}, NR^{b}R^{c}, C(═O)NR^{b}R^{c}, C(═S)NR^{b}R^{c}, SO₂NR^{b}R^{c}, OC(═O)R^{c}, OC(═O)OR^{e}, OC(═O)NR^{b}R^{e}, N(R^{c})C(═O)R^{c}, N(R^{c})C(═O)OR^{e}, N(R^{c})C(═O)NR^{b}R^{c}, NR^{c}SO₂R^{b}, NR^{c}SO₂NR^{b}R^{c}, Si(R^{d})₃, C(═NR^{c})R^{c}, C(NOR^{c})R^{c}′, C(═NNR^{b}R^{c})R^{c}, C(═NN(C(═O)R^{b})R^{c})R^{c}, C(═NN(C═O)OR^{c})(R^{c})₂, S(═O)ₒ(═NR^{b})_{q}R^{c} or N═CR^{b}R^{c};
R² and R³ are each independently selected from
C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with R^{a}; C(═O)R^{b}, C(═O)OR^{e}, NR^{b}R^{c}, C(═O)NR^{b}R^{c}, C(═S)NR^{b}R^{c}; C(═NR^{c})R^{c}, C(═NOR^{c})R^{c}′, C(═NNR^{b}R^{c})R^{c}, C(═NN(C(═O)R^{b})R^{c})R^{c}, C(═NN(C═O)OR^{c})(R^{c})₂,
or
OR^{c}, OC(═O)R^{c}, OC(═O)OR^{e}, OC(═O)NR^{b}R^{e},
NR^{b}R^{c}, N(R^{c})C(═O)R^{c}, N(R^{c})C(═O)OR^{e}, N(R^{c})C(═O)NR^{b}R^{c}, NR^{c}SO₂R^{b}, NR^{c}SO₂NR^{b}R^{c}, N═CR^{b}R^{c};
S(O)ₘR^{b}, SO₂NR^{b}R^{c}, S(═O)ₒ(═NR^{b})_{q}R^{c} or Si(R^{d})₃, or
a three- to eleven-membered saturated, or partially unsaturated or aromatic carbo- or heterocyclic ring or ring system, which may contain 1 to 4 heteroatoms selected from N(R^{c})ₚ, O, and S, wherein S may be oxidized, wherein the carbo- or heterocyclic rings system may be unsubstituted, partially or fully substituted with R^{a}; or
wherein one of R² and R³ is R⁴;
wherein one of R² and R³ is substituted with one R⁴;
or
R² and R³ form a four- to seven-membered ring, taken together with the carbon and nitrogen of the imidazole ring in formula (I), wherein each remaining ring member is selected from carbon atoms and up to 3 heteroatoms independently selected from up to 2 O, up to 2 S, and up to 3 N, wherein up to 2 carbon atom ring members are independently selected from C(═O) and C(═S), and the sulfur atom ring members are independently selected from S(═O)ₘ, wherein each ring member may be substituted with R^{a} and/or R^{c};
wherein the ring is substituted with one R⁴;
R⁴ is Het or R^{4a};
Het is a three- to ten-membered heterocyclic ring or a seven- to eleven-membered heterocyclic ring system, each ring or ring system member selected from carbon atoms and up to 4 heteroatoms independently selected from up to 2 O, up to 2 S, and up to 4 N(R^{c})ₚ, wherein up to 3 carbon atom ring members are independently selected from the group consisting of C(═O) and C(═S) and the sulfur atom ring members are independently selected from S(═O)^{o}(═NR^{b})_{q}, each ring or ring system optionally substituted with up to 5 R^{a};
o, q are each independently 0, 1 or 2, provided that the sum (o+q) is 0, 1 or 2 for each ring;
R^{4a} is C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₇-cycloalkyl, C₃-C₇-halocycloalkyl, C₄-C₈-alkylcycloalkyl, C₄-C₈-haloalkylcycloalkyl, C₄-C₈-cycloalkylalkyl, C₄-C₈-halocycloalkylalkyl, C₁-C₆-alkoxy, C₁-C₆-haloalkoxy, C₂-C₆-alkylcarbonyl, C₂-C₆-haloalkylcarbonyl, CN;
each optionally substituted with one or more substituents selected from CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b} C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b}SO₂R^{c}, NR^{b}SO₂NR^{b}R^{c}, SF₅, OCN, SCN, Si(R^{d})₃, C(═N(O)ₚR^{b})R^{b}, C(═NNR^{b}R^{c})R^{b}, C(═NN(C(═O)OₚR^{c})R^{b})R^{b}, ON═CR^{b}R^{c}, ONR^{b}R^{c}, S(═O)ₒ(═NR^{b})_{q}NR^{c}, SO₂NR^{b}(═O)NR^{b}R^{c}, P(═W)R^{b}R^{c}, OP(═W)(OₚR^{c})R^{b}, OP(═W)(OR^{c})₂, N═CR^{b}R^{c}, NR^{b}N═CR^{b}R^{c}, NR^{b}NR^{b}R^{c}, NR^{b}C(═S)NR^{b}R^{c}, NR^{b}C(═NR^{b})NR^{b}R^{c}, NR^{b}NR^{b}C(═W)NR^{b}R^{c}, NR^{b}NR^{b}SO₂NR^{b}R^{c}, or N═S(═O)ₚR^{c}R^{c}, or
two geminally bound groups R^{4a} together may form a group selected from the group consisting of ═O, ═S, ═CR^{b}R^{c}, ═NR^{c}, ═NOR^{c}, and ═NNR^{c}R^{c};
or R^{4a} is phenyl optionally substituted with one or more substituents selected from halogen, CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b} SO₂R^{c}, NR^{b}SO₂NR^{b}R^{c}, SF₅, OCN, SCN, Si(R^{d})₃, C(═N(O)ₚR^{b})R^{b}, C(═NNR^{b}R^{c})R^{b}, C(═NN(C(═O)OₚR^{c})R^{b})R^{b}, ON═CR^{b}R^{c}, ONR^{b}R^{c}, S(═O)ₒ(═NR^{b})_{q}R^{c}, SO₂NR^{b}(═O)NR^{b}R^{c}, P(═W)R^{b}R^{c}, OP(═W)(OₚR^{c})R^{b}, OP(═W)(OR^{c})₂, N═CR^{b}R^{c}, NR^{b}N═CR^{b}R^{c}, NR^{b}NR^{b}R^{c}, NR^{b}C(═S)NR^{b}R^{c}, NR^{b}C(═NR^{b})NR^{b}R^{c}, NR^{b}NR^{b}C(═W)NR^{b}R^{c}, NR^{b}NR^{b}SO₂NR^{b}R^{c}, or N═S(═O)ₚR^{c}R^{c},
or R^{4a} is phenyl optionally substituted with one or more substituents selected from C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₇-cycloalkyl, C₃-C₇-halocycloalkyl, C₄-C₈-alkylcycloalkyl, C₄-C₈-haloalkylcycloalkyl, C₄-C₈-cycloalkylalkyl, C₄-C₈-halocycloalkylalkyl, C₁-C₆-alkoxy, C₁-C₆-haloalkoxy, C₂-C₆-alkylcarbonyl, C₂-C₆-haloalkylcarbonyl, which groups may optionally be substituted with halogen, CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b}SO₂R^{c}, NR^{b}SO₂NR^{b}R^{c}, SF₅, OCN, SCN, Si(R^{d})₃, C(═N(O)ₚR^{b})R^{b}, C(═NNR^{b}R^{c})R^{b}, C(═NN(C(═O)OₚR^{c})R^{b}R^{b}, ON═CR^{b}R^{c}, ONR^{b}R^{c}, S(═O)ₒ(═NR^{b})_{q}NR^{c}, SO₂NR^{b}(═O)NR^{b}R^{c}, P(═W)R^{b}R^{c}, OP(═W)(OₚR^{c})R^{b}, OP(═W)(OR^{c})₂, N═CR^{b}R^{c}, NR^{b}N═CR^{b}R^{c}, NR^{b}NR^{b}R^{c}, NR^{b}C(═S)NR^{b}R^{c}, NR^{b}C(═NR^{b})NR^{b}R^{c}, NR^{b}NR^{b}C(═W)NR^{b}R^{c}, NR^{b}NR^{b}SO₂NR^{b}R^{c}, or N═S(═O)ₚR^{c}R^{c},
R^{a} is each independently halogen, C₁-C₆-alkyl, C₁-C₆-haloalkyl, C₁-C₆-alkoxy, C₁-C₆-haloalkoxy, C₂-C₄-alkenyl, C₂-C₄-alkynyl, C₃-C₆-cycloalkyl, CN, OR^{c}, NR^{b}R^{c}, NO₂, C(═O)(O)ₚR^{c}, OC(═O)(O)ₚR^{e}, C(═O)NR^{b}R^{c}, OC(═O)NR^{b}R^{e}, NR^{b}C(═O)(O)ₚR^{e}, NR^{b}C(═O)NR^{b}R^{c}, C(═S)NR^{b}R^{c}, S(O)ₘR^{b}, SO₂NR^{b}R^{c}, OSO₂R^{c}, OSO₂NR^{b}R^{c}, NR^{b}SO₂R^{c}, NR^{b}SO₂NR^{b}R^{c}, N═S(═O)ₚR^{c}R^{c}, S(═O)ₒ(═NR^{b})_{q}R^{c}, SF₅, OCN, SCN, Si(R^{d})₃ or a three- to six-membered saturated, or partially unsaturated or aromatic carbo- or heterocyclic ring, which may contain 1 to 3 heteroatoms selected from N—(R^{c})ₚ, O, and S which may be oxidized, and wherein the aforementioned groups and the carbo- or heterocyclic ring may be partially or fully substituted with R^{aa}, or
two geminally bound groups R^{a} together may form a group selected from the group consisting of ═O, ═S, ═CR^{b}R^{c}, ═NR^{c}, ═NOR^{c}, and ═NNR^{c}R^{c};
R^{aa} is each independently halogen, C₁-C₆-alkyl, C₁-C₆-haloalkyl, C₁-C₆-alkoxy or C₁-C₆-haloalkoxy;
R^{b} is each independently hydrogen, C₁-C₆-alkyl, C₁-C₆-haloalkyl, C₁-C₆-alkoxy, C₁-C₆-haloalkoxy or a three- to six-membered saturated, or partially unsaturated or aromatic carbo- or heterocyclic ring, which may contain 1 to 3 heteroatoms selected from the group consisting of N(R^{c})ₚ, O, and S, wherein S may be oxidized and which carbo- or heterocyclic ring may be partially or fully substituted with R^{aa};
R^{c} is each independently hydrogen, C₁-C₄-alkyl, C₁-C₄-haloalkyl, C₁-C₄-alkylcarbonyl, C₁-C₆ cycloalkyl, or a three- to six-membered saturated, partially unsaturated or aromatic carbo- or heterocyclic ring, which may contain 1 to 3 heteroatoms selected from N(R^{aa})ₚ, O and S, wherein S may be oxidized and wherein the carbo- or heterocyclic ring may be partially or fully substituted with R^{aa};
wherein two geminally bound groups R^{b}R^{b}, R^{c}R^{b} or R^{c}R^{c} together with the atom to which they are bound, may form a 3-, 4-, 5-, 6- or 7-membered saturated, partially unsaturated or aromatic carbo- or heterocyclic ring, which may contain 1 to 2 heteroatoms or heteroatoms groups selected from N, O, S, NO, SO and SO₂ and wherein the carbo- or heterocyclic ring may be partially or fully substituted with R^{a};
R^{d} is each independently hydrogen, phenyl, C₁-C₆-alkyl, C₂-C₆-alkenyl, C₂-C₆-alkynyl, C₃-C₈-cycloalkyl, or C₁-C₆-alkoxyalkyl, wherein the above mentioned groups may be substituted with one or more halogen;
R^{e} is each independently C₁-C₄-alkyl, C₁-C₄-haloalkyl, C₁-C₄-alkylcarbonyl, C₁-C₆ cycloalkyl, or a three- to six-membered saturated, partially unsaturated or aromatic carbo- or heterocyclic ring, which may contain 1 to 3 heteroatoms selected from N(R^{aa})ₚ, O and S, wherein S may be oxidized and wherein the carbo- or heterocyclic ring may be partially or fully substituted with R^{aa};
m is 0, 1, or 2;
n is 0, 1 or 2;
p is 0 or 1;
R⁵ is hydrogen, C₁-C₆-alkyl, C₂-C₆-alkenyl, C₂-C₆-alkynyl, which groups may be independently from each other substituted with one or up to five halogen or with one NO₂, CN, C₃-C₆-cycloalkyl, O—R⁵¹, —S(O)_{q}—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl,
wherein aryl, hetaryl, heterocyclyl or oxoheterocyclyl may in turn be substituted with one or up to three halogen, NO₂, CN, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, C₁-C₆-alkylcarbonylamino, aryl or hetaryl;
wherein aryl and hetaryl may be substituted with one or more, identical or different, halogen, CN, NO₂, hydroxy, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio;
or
R⁵ is C₃-C₆-cycloalkyl, C₃-C₆-heterocyclyl, which groups may be independently from each other substituted with one to three halogen or one CN, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulfinyl, C₁-C₆-alkylsulfonyl, aryl, hetaryl, aryl-C₁-C₆-alkyl or hetaryl-C₁-C₆-alkyl,
wherein aryl, hetaryl, aryl-C₁-C₆-alkyl and hetaryl-C₁-C₆-alkyl may be substituted with one to three halogen, CN, NO₂, C₁-C₄-alkyl, C₁-C₄-haloalkyl, C₁-C₄-alkoxy, C₁-C₄-haloalkoxy;
or
R⁵ is aryl or hetaryl, which groups may be independently from each other substituted with one to three halogen, NO₂, amino, CN, SF₅, SCN, C₁-C₆-alkylamino, di-(C₁-C₆)-alkylamino, hydroxy, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, or tri-(C₁-C₆-alkyl)-silyl;
q is 0, 1 or 2;
R⁵¹ is hydrogen, C₁-C₆-alkyl, C₂-C₆-alkenyl, C₂-C₆-alkynyl, C₃-C₆-cycloalkyl or C₃-C₆-heterocyclyl, which groups may be independently from each other substituted with one to three halogen or one NO₂, CN, C₃-C₆-cycloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, aryl or hetaryl, wherein aryl and hetaryl may be substituted with one to three halogen, CN, NO₂, C₁-C₄-alkyl, C₁-C₄-haloalkyl, C₁-C₄-alkoxy or C₁-C₄-haloalkoxy;
or
R⁵¹ is aryl or hetaryl, which groups may be independently from each other substituted with one to three halogen, NO₂, amino, CN, SF₅, SCN, C₁-C₆-alkylamino, di-(C₁-C₆)-alkylamino, hydroxy, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, or tri-(C₁-C₆-alkyl)-silyl;
R⁵² is C₁-C₆-alkyl, C₂-C₆-alkenyl, C₂-C₆-alkynyl, C₃-C₆-cycloalkyl or C₃-C₆-heterocyclyl, which groups may be independently from each other substituted with one to three halogen, NO₂, CN, C₃-C₆-cycloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, aryl or hetaryl, wherein aryl and hetaryl may be substituted with one to three halogen, CN, NO₂, C₁-C₄-alkyl, C₁-C₄-haloalkyl, C₁-C₄-alkoxy or C₁-C₄-haloalkoxy;
or
R⁵² is aryl or hetaryl, which groups may be independently from each other substituted with one to three halogen, NO₂, amino, CN, SF₅, SCN, C₁-C₆-alkylamino, di-(C₁-C₆)-alkylamino, hydroxy, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulfonyl, C₁-C₆-haloalkylsulfonyl, C₁-C₆-alkylaminocarbonyl, di-(C₁-C₆)-alkylaminocarbonyl, or tri-(C₁-C₆-alkyl)-silyl;
R⁵³ is hydrogen, C₁-C₆-alkyl, C₂-C₆-alkenyl, C₂-C₆-alkynyl, C₃-C₆-cycloalkyl, C₁-C₆-alkoxy, C₁-C₆-alkoxy-C₁-C₆-alkyl, C₁-C₆-alkoxycarbonyl, C₁-C₆-alkylcarbonyl, aryl, hetaryl, arylcarbonyl or hetarylcarbonyl, wherein aryl and hetaryl may be substituted with one to three halogen, CN, NO₂, C₁-C₄-alkyl, C₁-C₄-haloalkyl, C₁-C₄-alkoxy or C₁-C₄-haloalkoxy;
R⁵⁴ is hydrogen, C₁-C₆-alkyl, C₂-C₆-alkenyl, C₂-C₆-alkynyl, C₃-C₆-cycloalkyl or C₃-C₆-cycloalkyl-C₁-C₆-alkyl, which groups may be independently from each other substituted with one to five halogen or one CN, NO₂, hydroxy, C₁-C₆-alkyl, C₃-C₆-cycloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-alkoxycarbonyl, C₁-C₆-alkylcarbonyl or tri-(C₁-C₆-alkyl)-silyl;
or
R⁵⁴ is aryl, hetaryl, aryl-C₁-C₆-alkyl or hetaryl-C₁-C₆-alkyl, which groups may be independently from each other substituted with halogen, CN, NO₂, hydroxy, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₂-C₆-alkoxycarbonyl or C₂-C₆-alkylcarbonyl,
or
R⁵³ and R⁵⁴ are connected through two to six carbon atoms and form a ring, which may comprise an additional atom selected from O, S or N, and which may be substituted with one to four C₁-C₂-alkyl, halogen, CN, amino or C₁-C₂-alkoxy;
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, wherein aryl, hetaryl, aryl-C₁-C₆-alkyl and hetaryl-C₁-C₆-alkyl, may be substituted with one or more, identical or different, halogen, CN, NO₂, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl;
R⁵⁶ is C₁-C₆-alkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, wherein aryl, hetaryl and aryl-C₁-C₆-alkyl may be substituted with one or more, identical or different, halogen, CN, NO₂, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl;
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, wherein aryl, hetaryl, aryl-C₁-C₆-alkyl and hetaryl-C₁-C₆-alkyl, may be substituted with one or more, identical or different, halogen, CN, NO₂, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl;
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, or C₁-C₆-alkylcarbonyl;
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl, or C₁-C₆-alkylcarbonyl;
R⁸ is hydrogen, C₁-C₆-alkyl, C₂-C₆-alkenyl, C₂-C₆-alkynyl, which groups may be independently from each other substituted with one to five halogen or one CN, NO₂, hydroxy, C₁-C₆-alkyl, C₃-C₆-cycloalkyl, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₁-C₄-alkylsulfimino-C₁-C₄-alkyl, C₁-C₄-alkylsulfimino-C₂-C₅-alkylcarbonyl, C₁-C₄-alkylsulfoximino, C₁-C₄-alkylsulfoximino-C₁-C₄-alkyl, C₁-C₄-alkylsulfoximino-C₂-C₅-alkylcarbonyl, C₁-C₆-alkoxycarbonyl, C₁-C₆-alkylcarbonyl or C₃-C₆-trialkylsilyl;
or
R⁸ is aryl-C₁-C₆-alkyl or hetaryl-C₁-C₆-alkyl, C₃-C₁₂-cycloalkyl, C₃-C₁₂-cycloalkyl-C₁-C₆-alkyl or C₄-C₁₂-bicycloalkyl, which groups may be substituted with halogen, CN, NO₂, hydroxy, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₂-C₆-alkoxycarbonyl or C₂-C₆-alkylcarbonyl,
or
R⁸ is a five- to six-membered aromatic or heteroaromatic ring which may be substituted with one or more identical or different substituents, a four- to six-membered partially saturated ring, a saturated heterocyclic ring, or a saturated or aromatic heterobicyclic ring which may comprise one to three heteroatoms from O, S or N and which may be substituted with one or more substituents, wherein the substituents are independently from each other halogen, CN, NO₂, hydroxy, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulfinyl, C₁-C₄-alkylsulfonyl, C₁-C₄-alkylsulfimino, C₂-C₆-alkoxycarbonyl or C₂-C₆-alkylcarbonyl,
or
R⁷ and R⁸ are connected through two to six carbon atoms and form a ring, which may comprise an additional atom selected from O, S or N, and which may be substituted with one to four C₁-C₂-alkyl, halogen, CN, amino or C₁-C₂-alkoxy;
or a stereoisomer, tautomer, salt, or N-oxide thereof.

17. The compound of formula (I) of claim 16, wherein
R¹ is C₁-C₄-alkyl, C₃-C₆-cycloalkyl, C₂-C₄-alkenyl, benzyl or phenyl, which groups may be partially or fully substituted with halogen or C₁-C₄-alkyl.

18. The compound of formula (I) of claim 16, wherein R² and R³ are each independently selected from C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with R^{a}; OR^{c}, OC(═O)R^{c}, NR^{b}R^{c}, S(O)ₘR^{b}.

19. The compound of formula (I) of claim 16 or a stereoisomer, tautomer, salt, or N-oxide thereof, wherein R³ is substituted with one R⁴.

20. The compound of formula (I) of claim 16 or a stereoisomer, tautomer, salt, or N-oxide thereof, wherein
R² and R³ form a four- to seven-membered ring, taken together with the carbon and nitrogen of the imidazole ring in formula (I), wherein each remaining ring member is selected from carbon atoms and up to 3 heteroatoms independently selected from up to 2 O, up to 2 S, and up to 3 N, wherein up to 2 carbon atom ring members are independently selected from the group consisting of C(═O) and C(═S), and the sulfur atom ring members are independently selected from S(═O)ₘ, wherein each ring member may be substituted with R^{a} and/or R^{c}; and
wherein the ring is substituted with one R⁴.

21. The compound of formula (I) of claim 16, wherein
R² and R³, together with the nitrogen and the carbon atom of the imidazolium ring, form a five or six membered ring resulting in the compounds of formula (II) selected from the group consisting of compounds of formulae II-1 to II-16:

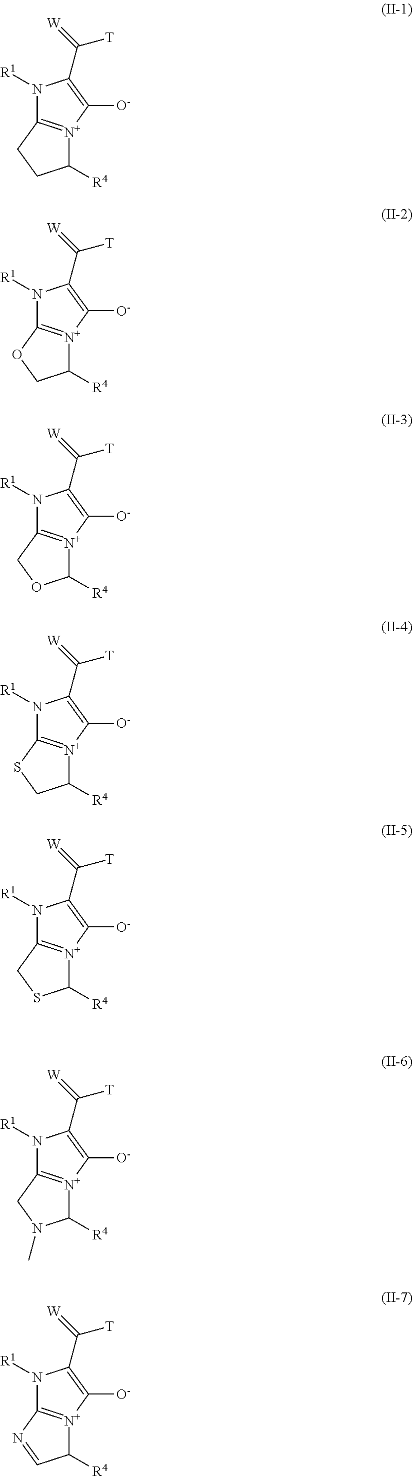

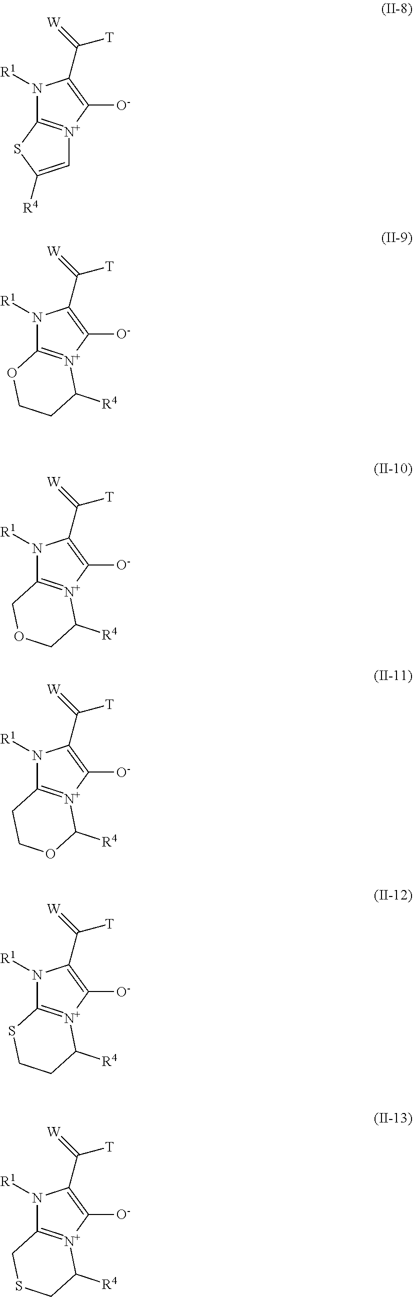

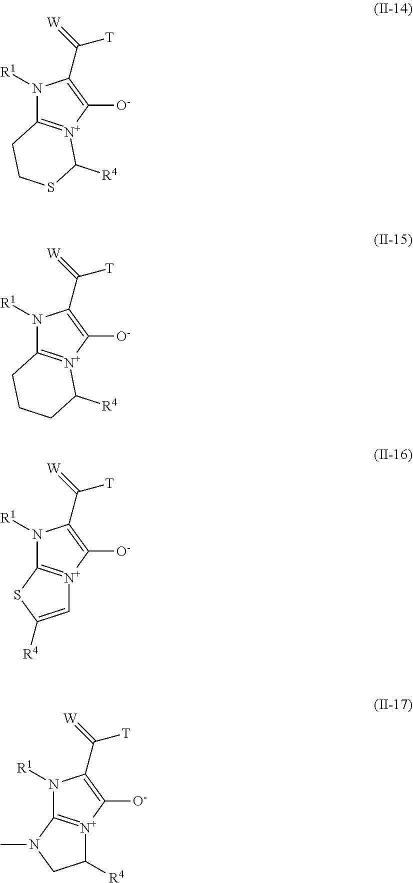

22. The compound of formula (I) of claim 16, wherein
R⁴ is CH₂CN, CH₂CH₂CN, or Het, wherein Het is selected from the group consisting of D-1 to D-56:

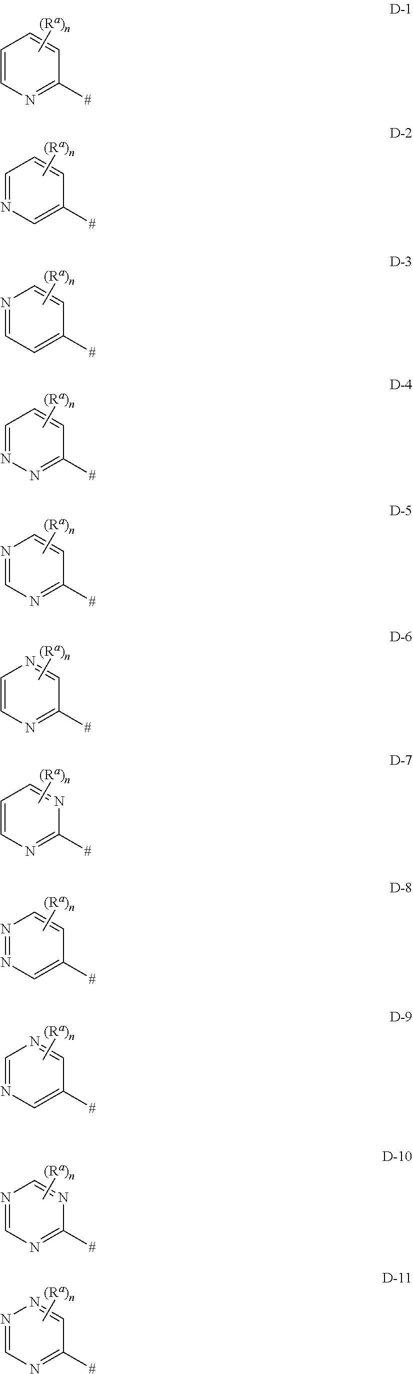

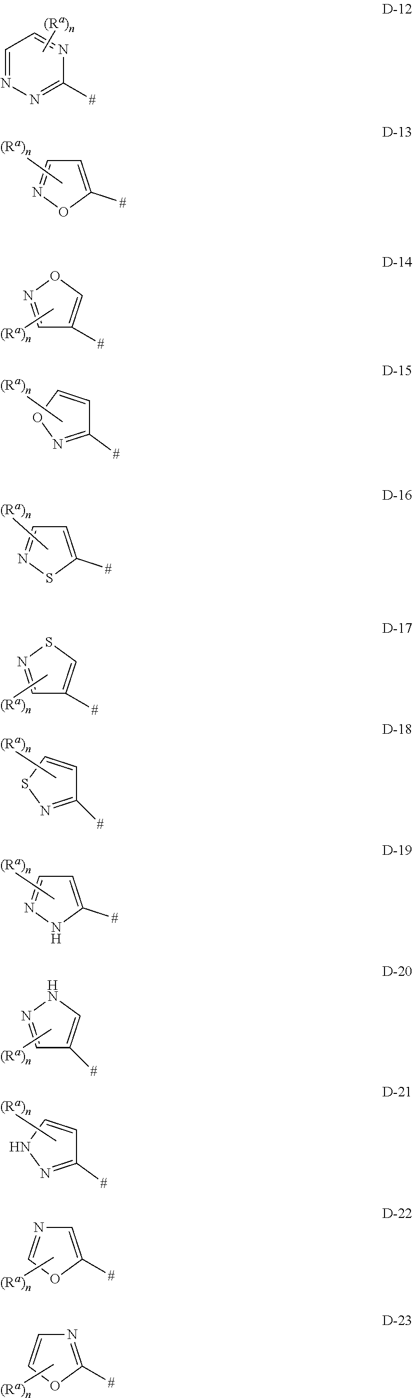

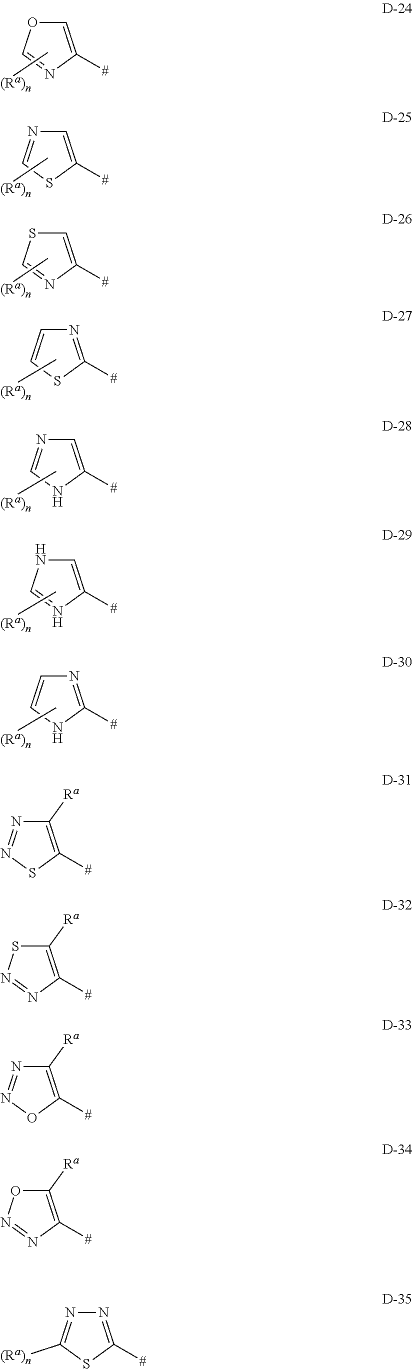

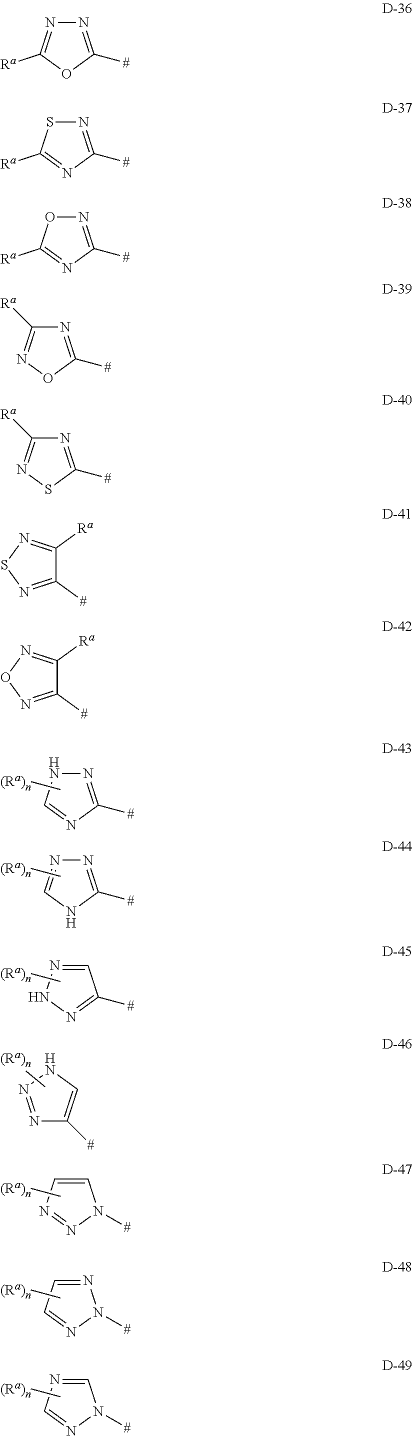

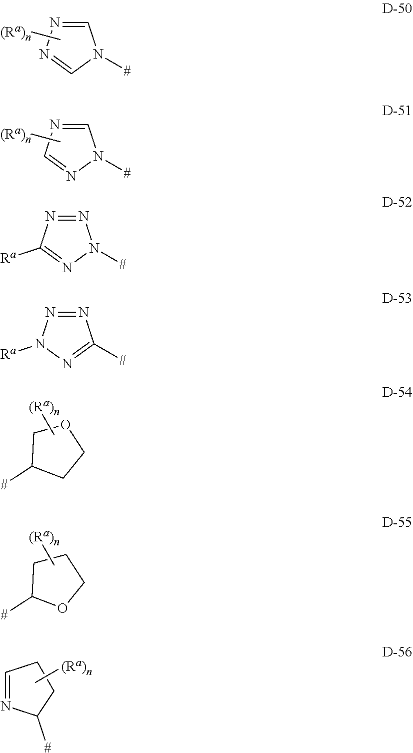

23. The compound of formula (I) of claim 16, wherein
W is O, and T is C₁-C₆-alkyl, C₂-C₆-alkenyl, C₂-C₆-alkynyl, each optionally substituted with one or up to five halogen or with one NO₂, CN, C₃-C₆-cycloalkyl, O—R⁵¹, —S(O)_{q}—R⁵², or —N(R⁵³)(R⁵⁴).

24. A composition comprising at least one compound of formula (I) of claim 16 and at least one inert liquid and/or solid carrier.

25. A method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing with a pesticidally effective amount of at least one compound of formula (I) of claim 16 or a stereoisomer, tautomer, salt, or N-oxide thereof.

26. A method for combating, controlling, preventing or protecting against infestation or infection by invertebrate pest, which method comprises contacting said pest or its food supply, habitat or breeding grounds with a pesticidally effective amount of at least one compound of formula (I) of claim 16 or a stereoisomer, tautomer, salt, or N-oxide thereof.

27. A non-therapeutic method for treating animals infested or infected by parasites or preventing animals of getting infected or infested by parasites or protecting animals against infestation or infection by parasites which comprises orally, topically or parenterally administering or applying to the animals a parasiticidally effective amount of a compound of formula (I) of claim 16 or a stereoisomer, tautomer, salt, or N-oxide thereof.

28. A seed comprising a compound of formula (I) of claim 16 or a stereoisomer, tautomer, salt, or N-oxide thereof in an amount of from 0.1 g to 10 kg per 100 kg of seed.

29. The compound of formula (I) of claim 22, wherein Het is selected from the group consisting of the following rings systems D-2a, D-2b, D-2c, D-25a, preferably D-25a substituted with Cl, D-56 and D-56a:

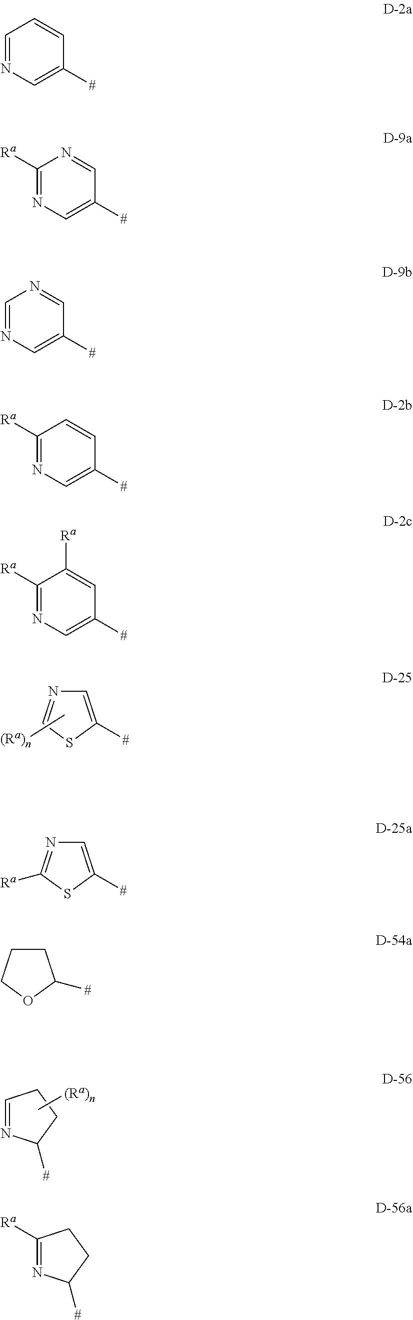

30. A method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing with a composition of claim 24.

31. The compound of formula (I) of claim 16, wherein R² and R³ are each independently selected from C₁-C₈-alkyl, C₂-C₈-alkenyl, C₂-C₈-alkynyl, C₃-C₁₀-cycloalkyl, C₄-C₁₀-cycloalkenyl, which groups may be unsubstituted, partially or fully substituted with R^{a}; OR^{c}, OC(═O)R^{c}, NR^{b}R^{c}, S(O)ₘR^{b}.

32. The compound of formula (I) of claim 17 or a stereoisomer, tautomer, salt, or N-oxide thereof, wherein R³ is substituted with one R⁴.

33. The compound of formula (I) of claim 17 or a stereoisomer, tautomer, salt, or N-oxide thereof, wherein
R² and R³ form a four- to seven-membered ring, taken together with the carbon and nitrogen of the imidazole ring in formula (I), wherein each remaining ring member is selected from carbon atoms and up to 3 heteroatoms independently selected from up to 2 O, up to 2 S, and up to 3 N, wherein up to 2 carbon atom ring members are independently selected from the group consisting of C(═O) and C(═S), and the sulfur atom ring members are independently selected from S(═O)ₘ, wherein each ring member may be substituted with R^{a} and/or R^{c}; and
wherein the ring is substituted with one R⁴.

34. The compound of formula (I) of claim 17, wherein
R² and R³, together with the nitrogen and the carbon atom of the imidazolium ring, form a five or six membered ring resulting in the compounds of formula (II) selected from the group consisting of compounds of formulae II-1 to II-16:

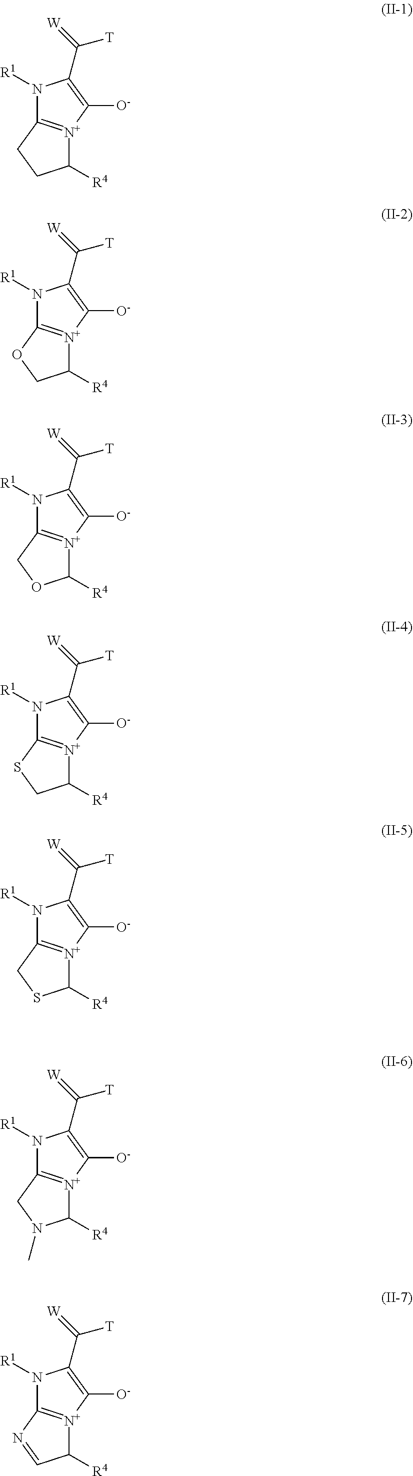

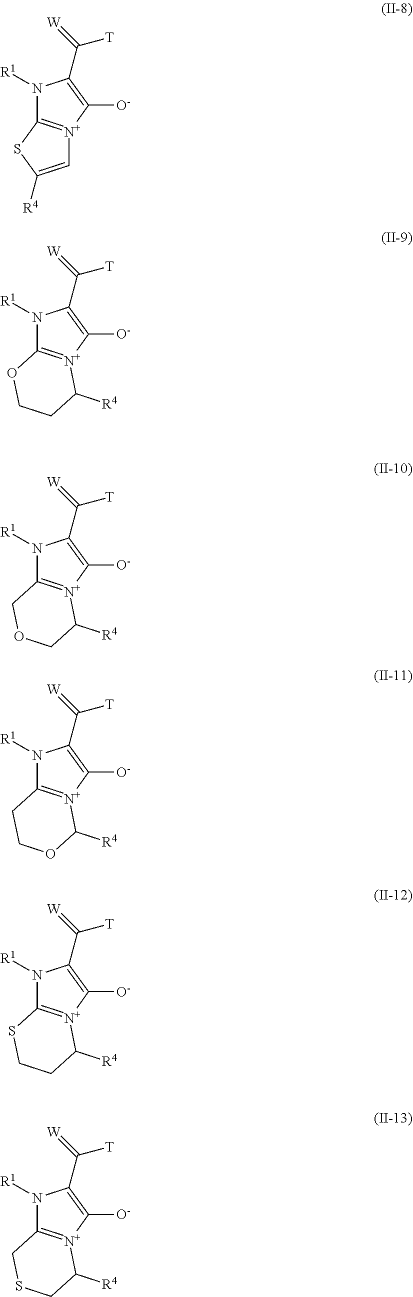

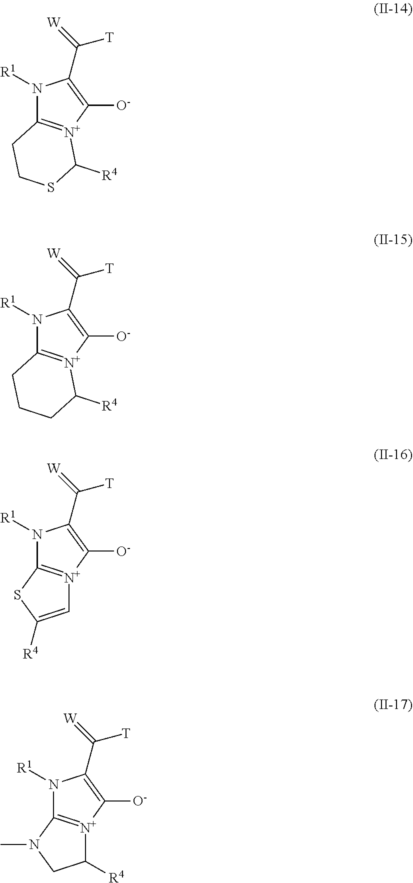

35. The compound of formula (I) of claim 17, wherein
R² and R³, together with the nitrogen and the carbon atom of the imidazolium ring, form a five or six membered ring resulting in the compounds of formula (II) selected from the group consisting of compounds of formulae II-1 to II-16: 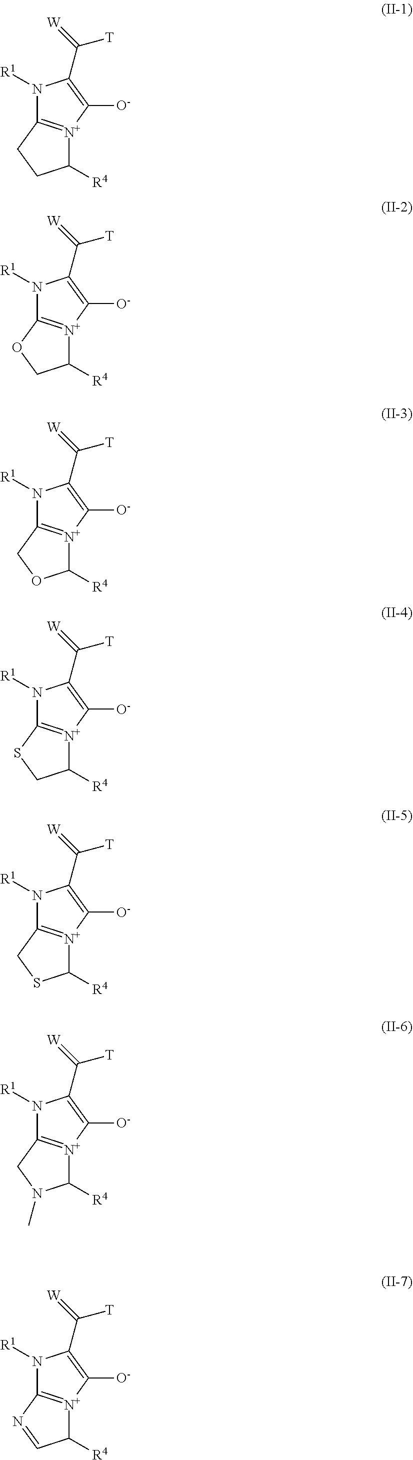 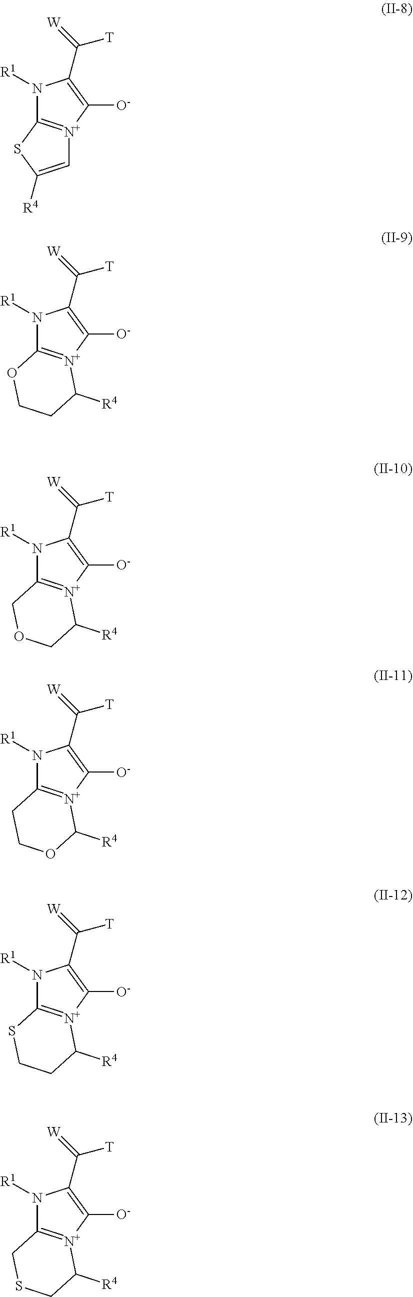 [chemical structure]
